SEQUENCE LISTING (69 sequences)

SEQ ID NO: 1 - DNA, 2129 bp, Arabidopsis thaliana
ataaccttcctctctatttttacaatttattttgttattagaagtggtagtggagtgaaaaaacaaatcctaagcagtcctaaccgatccccgaagctaaagattcttcaccttcccaaataaagcaaaacctagatccgacattgaaggaaaaaccttttagatccatctctgaaaaaaaaccaaccatgaagagagatcatcatcatcatcatcatcaagataagaagactatgatgatgaatgaagaagacgacggtaacggcatggatgagcttctagctgttcttggttacaaggttaggtcatccgaaatggctgatgttgctcagaaactcgagcagcttgaagttatgatgtctaatgttcaagaagacgatctttctcaactcgctactgagactgttcactataatccggcggagctttacacgtggcttgattctatgctcaccgaccttaatcctccgtcgtctaacgccgagtacgatcttaaagctattcccggtgacgcgattctcaatcagttcgctatcgattcggcttcttcgtctaaccaaggcggcggaggagatacgtatactacaaacaagcggttgaaatgctcaaacggcgtcgtggaaaccactacagcgacggctgagtcaactcggcatgttgtcctggttgactcgcaggagaacggtgtgcgtctcgttcacgcgcttttggcttgcgctgaagctgttcagaaagagaatctgactgtagcggaagctctggtgaagcaaatcggattcttagccgtttctcaaatcggagcgatgagaaaagtcgctacttacttcgccgaagctctcgcgcggcggatttaccgtctctctccgtcgcagagtccaatcgaccactctctctccgatactcttcagatgcacttctacgagacttgtccttatctcaagttcgctcacttcacggcgaatcaagcgattctcgaagcttttcaagggaagaaaagagttcatgtcattgatttctctatgagtcaaggtcttcaatggccggcgcttatgcaggctcttgcgcttcgacctggtggtcctcctgttttccggttaaccggaattggtccaccggcaccggataatttcgattatcttcatgaagttgggtgtaagctggctcatttagctgaggcgattcacgttgagtttgagtacagaggatttgtggctaacactttagctgatcttgatgcttcgatgcttgagcttagaccaagtgagattgaatctgttgcggttaactctgttttcgagcttcacaagctcttgggacgacctggtgcgatcgataaggttcttggtgtggtgaatcagattaaaccggagattttcactgtggttgagcaggaatcgaaccataatagtccgattttcttagatcggtttactgagtcgttgcattattactcgacgttgtttgactcgttggaaggtgtaccgagtggtcaagacaaggtcatgtcggaggtttacttgggtaaacagatctgcaacgttgtggcttgtgatggacctgaccgagttgagcgtcatgaaacgttgagtcagtggaggaaccggttcgggtctgctgggtttgcggctgcacatattggttcgaatgcgtttaagcaagcgagtatgcttttggctctgttcaacggcggtgagggttatcgggtggaggagagtgacggctgtctcatgttgggttggcacacacgaccgctcatagccacctcggcttggaaactctccaccaattagatggtggctcaatgaattgatctgttgaaccggttatgatgatagatttccgaccgaagccaaactaaatcctactgtttttccctttgtcacttgttaagatcttatctttcattatattaggtaattgaaaaattttaatctcgctttggagagttttttttttttgcatgtgacattggagggtaaattggataggcagaaatagaagtatgtgttaccaagtatgtgcaattggttgaaataaaatcatcttgagtgtcaccatctataaaattcattgtaatgactaatgagcctgattaaactgtctcttatgataatgtgctgattctcatg

SEQ ID NO: 2 - protein, 533 aa, Arabidopsis thaliana
MKRDHHHHHHQDKKTMMMNEEDDGNGMDELLAVLGYKVRSSEMADVAQKLEQLEVMMSNVQEDDLSQLATETVHYNPAELYTWLDSMLTDLNPPSSNAEYDLKAIPGDAILNQFAIDSASSSNQGGGGDTYTTNKRLKCSNGVVETTTATAESTRHVVLVDSQENGVRLVHALLACAEAVQKENLTVAEALVKQIGFLAVSQIGAMRKVATYFAEALARRIYRLSPSQSPIDHSLSDTLQMHFYETCPYLKFAHFTANQAILEAFQGKKRVHVIDFSMSQGLQWPALMQALALRPGGPPVFRLTGIGPPAPDNFDYLHEVGCKLAHLAEAIHVEFEYRGFVANTLADLDASMLELRPSEIESVAVNSVFELHKLLGRPGAIDKVLGVVNQIKPEIFTVVEQESNHNSPIFLDRFTESLHYYSTLFDSLEGVPSGQDKVMSEVYLGKQICNVVACDGPDRVERHETLSQWRNRFGSAGFAAAHIGSNAFKQASMLLALFNGGEGYRVEESDGCLMLGWHTRPLIATSAWKLSTN

SEQ ID NO: 3 - DNA, 2302 bp, Arabidopsis thaliana
atgaatgatgattgaagtggtagtagcagtgaaaaacaaaagcaatccaatcccaaacccatttgctcttaagattcttcacatagagaagtcacatgttccttcttcttcttccttcatcatccccaaacacacacaaactaaaaaaaaggcaaaaccctagatccaagatcagacctaatctaatcgaaactcatagctgaaaaatgaagagagatcatcaccaattccaaggtcgattgtccaaccacgggacttcttcttcatcatcatcaatctctaaagataagatgatgatggtgaaaaaagaagaagacggtggaggtaacatggacgacgagcttctcgctgttttaggttacaaagttaggtcatcggagatggcggaggttgctttgaaactcgaacaattagagacgatgatgagtaatgttcaagaagatggtttatctcatctcgcgacggatactgttcattataatccgtcggagctttattcttggcttgataatatgctctctgagcttaatcctcctcctcttccggcgagttctaacggtttagatccggttcttccttcgccggagatttgtggttttccggcttcggattatgaccttaaagtcattcccggaaacgcgatttatcagtttccggcgattgattcttcgtcttcgtcgaataatcagaacaagcgtttgaaatcatgctcgagtcctgattctatggttacatcgacttcgacgggtacgcagattggtggagtcataggaacgacggtgacgacaaccaccacgacaacgacggcggcgggtgagtcaactcgttctgttatcctggttgactcgcaagagaacggtgttcgtttagtccacgcgcttatggcttgtgcagaagcaatccagcagaacaatttgactctagcggaagctcttgtgaagcaaatcggatgcttagctgtgtctcaagccggagctatgagaaaagtggctacttacttcgccgaagctttagcgcggcggatctaccgtctctctccgccgcagaatcagatcgatcattgtctctccgatactcttcagatgcacttttacgagacttgtccttatcttaaattcgctcacttcacggcgaaccaagcgattctcgaagcttttgaaggtaagaagagagtacacgtcattgatttctcgatgaaccaaggtcttcaatggcctgcacttatgcaagctcttgcgcttcgagaaggaggtcctccaactttccggttaaccggaattggtccaccggcgccggataattctgatcatcttcatgaagttggttgtaaattagctcagcttgcggaggcgattcacgtagaattcgaataccgtggattcgttgctaacagcttagccgatctcgatgcttcgatgcttgagcttagaccgagcgatacggaagctgttgcggtgaactctgtttttgagctacataagctcttaggtcgtcccggtgggatagagaaagttctcggcgttgtgaaacagattaaaccggtgattttcacggtggttgagcaagaatcgaaccataacggaccggttttcttagaccggtttactgaatcgttacattattattcgactctgtttgattcgttggaaggagttccgaatagtcaagacaaagtcatgtctgaagtttacttagggaaacagatttgtaatctggtggcttgtgaaggtcctgacagagtcgagagacacgaaacgttgagtcaatggggaaaccggtttggttcgtccggtttagcgccggcacatcttgggtctaacgcgtttaagcaagcgagtatgcttttgtctgtgtttaatagtggccaaggttatcgtgtggaggagagtaatggatgtttgatgttgggttggcacactcgtccactcattaccacctccgcttggaaactctcgacggcggcgtactgagtttgactcgaagcatacgacggtggtggagtcgagtcgagtgaatttgagattgagatcagtggaccggtgatgacatatgttcggaccaagacctaaaccgaactgaatcgaaccgttttgccttttgtttattttatttattttcgttcacttgtttaaaattcttatatatatcgttttggtaggtcatttttaatttatgcctttttgggatcaatttttaataggctgagtttgtatttattaataaattatctttatgaattttaaactaaaactatgttttaatctcatttaaaaaaaaattaatatcaagttttattaatctc

SEQ ID NO: 4 - protein, 587 aa, Arabidopsis thaliana
MKRDHHQFQGRLSNHGTSSSSSSISKDKMMMVKKEEDGGGNMDDELLAVLGYKVRSSEMAEVALKLEQLETMMSNVQEDGLSHLATDTVHYNPSELYSWLDNMLSELNPPPLPASSNGLDPVLPSPEICGFPASDYDLKVIPGNAIYQFPAIDSSSSSNNQNKRLKSCSSPDSMVTSTSTGTQIGGVIGTTVTTTTTTTTAAGESTRSVILVDSQENGVRLVHALMACAEAIQQNNLTLAEALVKQIGCLAVSQAGAMRKVATYFAEALARRIYRLSPPQNQIDHCLSDTLQMHFYETCPYLKFAHFTANQAILEAFEGKKRVHVIDFSMNQGLQWPALMQALALREGGPPTFRLTGIGPPAPDNSDHLHEVGCKLAQLAEAIHVEFEYRGFVANSLADLDASMLELRPSDTEAVAVNSVFELHKLLGRPGGIEKVLGVVKQIKPVIFTVVEQESNHNGPVFLDRFTESLHYYSTLFDSLEGVPNSQDKVMSEVYLGKQICNLVACEGPDRVERHETLSQWGNRFGSSGLAPAHLGSNAFKQASMLLSVFNSGQGYRVEESNGCLMLGWHTRPLITTSAWKLSTAAY

SEQ ID NO: 5 - DNA, 1849 bp, Arabidopsis thaliana
atatcattatttaaaaatagaattttatttttctttcttcttcttcaattattatgacactcccgtgttcctaatcttttctcttattcttctctttcttctcatcttacaaaatcttgcaaatcaattttaatgaagagagagcacaaccaccgtgaatcatccgccggagaaggtgggagttcatcaatgacgacggtgattaaagaagaagctgccggagttgacgagcttttggttgttttaggttacaaagttcgatcatccgacatggctgacgtggcacacaagcttgaacagttagagatggttcttggtgatggaatctcgaatctttctgatgaaactgttcattacaatccttctgatctctctggttgggtcgaaagcatgctctcggatcttgacccgacccggattcaagaaaagcctgactcagagtacgatcttagagctattcctggctctgcagtgtatccacgtgacgagcacgtgactcgtcggagcaagaggacgagaattgaatcggagttatcctctacgcgctctgtggtggttttggattctcaagaaactggagtgcgtttagtccacgcgctattagcttgtgctgaagctgttcaacagaacaatttgaagttagccgacgcgctcgtgaagcacgtggggttactcgcgtcctctcaagctggtgctatgaggaaagtcgcgacttacttcgctgaagggcttgcgagaaggatttaccgtatttaccctcgagacgatgtcgcgttgtcttcgttttcggacactcttcagattcatttctatgagtcttgtccgtatctcaagtttgcgcattttacggcgaatcaagcgatacttgaggtttttgctacggcggagaaggttcatgttattgatttaggacttaaccatggtttacaatggccggctttgattcaagctcttgctttacgtcctaatggtccaccggattttcggttaaccgggatcggttattcgttaaccgatattcaagaagttggttggaaacttggtcagcttgcgagtactattggtgtcaatttcgaattcaagagcattgctttaaacaatttgtctgatcttaaaccggaaatgctagacattagacccggtttagaatcagtggcggttaactcggtcttcgagcttcatcgcctcttagctcatcccggttccatcgataagtttttatcgacaatcaaatcaatccgaccggatataatgactgtggtcgagcaagaagcaaaccataacggtaccgtatttctcgatcggttcacggaatcgctacattactattcgagcttattcgactcgctcgagggcccgccaagccaagaccgagtgatgtcggagttattcctaggacggcagatactaaaccttgtggcatgcgaaggagaagaccgggtagagaggcatgagactttaaatcagtggagaaaccggttcggtttaggaggatttaaaccggttagtatcggttcgaacgcgtataagcaagcaagcatgttgttggcactttatgccggggctgatgggtataatgtggaagagaatgaaggttgtttgttgcttggatggcaaacgcgaccgcttattgcaacatctgcgtggcgaatcaatcgtgtggaataaaaataaataatgggaaaagtgaaaatgtgctatatactttattgcattgctgataaagaaaaaaagtcccacgttttccaaattttatgaattctaaattttgttcacttgtcacgagattttgacctcgcataaatagactattacgtcagggtcaggccaatgaaatgattttttatca

SEQ ID NO: 6 - protein, 511 aa, Arabidopsis thaliana
MKREHNHRESSAGEGGSSSMTTVIKEEAAGVDELLVVLGYKVRSSDMADVAHKLEQLEMVLGDGISNLSDETVHYNPSDLSGWVESMLSDLDPTRIQEKPDSEYDLRAIPGSAVYPRDEHVTRRSKRTRIESELSSTRSVVVLDSQETGVRLVHALLACAEAVQQNNLKLADALVKHVGLLASSQAGAMRKVATYFAEGLARRIYRIYPRDDVALSSFSDTLQIHFYESCPYLKFAHFTANQAILEVFATAEKVHVIDLGLNHGLQWPALIQALALRPNGPPDFRLTGIGYSLTDIQEVGWKLGQLASTIGVNFEFKSIALNNLSDLKPEMLDIRPGLESVAVNSVFELHRLLAHPGSIDKFLSTIKSIRPDIMTVVEQEANHNGTVFLDRFTESLHYYSSLFDSLEGPPSQDRVMSELFLGRQILNLVACEGEDRVERHETLNQWRNRFGLGGFKPVSIGSNAYKQASMLLALYAGADGYNVEENEGCLLLGWQTRPLIATSAWRINRVE

SEQ ID NO: 7 - DNA, 2070 bp, Arabidopsis thaliana
caaatcccattaataaaaaccttaccaacccatgaagtaaagtaaactcctttcttataaactctcttttgttctttttttttcaacttcatcagtctcttaactcaccatcacaagaacaagaaagatgaagagaggatacggagaaacatgggatccgccaccaaaaccactaccagcttctcgttccggagaaggtccttcaatggcggataagaagaaggctgatgatgacaacaacaacagcaacatggatgatgagcttcttgctgttcttggctacaaggttcgatcttctgagatggctgaagtagcacagaagcttgaacaacttgagatggtcttgtctaatgatgatgttggttctactgtcttaaacgactctgttcattataacccatctgatctctctaactgggtcgagagcatgctttctgagctgaacaacccggcttcttcggatcttgacacgacccgaagttgtgtggatagatccgaatacgatctcagagcaattccgggtctttctgcgtttccaaaggaagaggaagtctttgacgaggaagctagcagcaagaggatccgactcggatcgtggtgcgaatcgtcggacgagtcaactcggtccgtggtgctcgttgactctcaggagaccggagttagacttgtccacgcactagtggcgtgcgctgaggcgattcaccaggagaatctcaacttagctgacgcgctggtgaaacgcgtgggaacactcgcgggttctcaagctggagctatgggaaaagtcgctacgtattttgctcaagccttggctcgtcgtatttaccgtgattacacggcggagacggacgtttgcgcggcggtgaacccatctttcgaagaggttttggagatgcacttttacgagtcttgcccttacctgaagttcgctcatttcacggcgaaccaagcgattctagaagctgttacgacggcgcgtagagttcacgtcattgatttagggcttaatcaagggatgcaatggcctgctttaatgcaagctttagctctccgacccggtggacctccgtcgtttcgtctcaccggaatcggaccaccgcagacggagaattcagattcgcttcaacagttaggttggaaattagctcaattcgctcagaacatgggcgttgaattcgaattcaaaggcttagccgctgagagtttatcggatcttgaacccgaaatgttcgaaacccgacccgaatctgaaaccttagtggttaattcggtatttgagctccaccggttattagcccgatccggttcaatcgaaaagcttctcaatacggttaaagctattaaaccgagtatcgtaacggtggttgagcaagaagcgaaccacaacggaatcgtcttcctcgataggttcaacgaagcgcttcattactactcgagcttgtttgactcgctcgaagacagttatagtttaccgagtcaagaccgagttatgtcagaagtgtacttagggagacagatactcaacgttgttgcggcggaagggtccgatcgggtcgagcggcacgagacggctgcacagtggaggattcggatgaaatccgctgggtttgacccgattcatctcggatctagcgcgtttaaacaagcgagtatgcttttatcgctttacgctaccggagatggatacagagttgaagaaaatgacggatgtttaatgatagggtggcagacgcgaccactcatcacaacctcggcgtggaaactcgcctgagtcgcggcggtagagatgactcgcctgaaaccgggaaaaacaataaatgttttaaaaaattaggaaaagagaccgtaactttagttatgtttttactttttaacccgaagtttttgtgtgtttaacctttttgcctaaatgtttacaactttatctttttggaccttgtgcgtatctttgagagttaagagaacgagtaaaaaatcttgtatcgtagatcgagctaagtagttttcaataaatggaaggataacgattctgtatgttttttacttgatccaatatatatgaatttattt

SEQ ID NO: 8 - protein, 547 aa, Arabidopsis thaliana
MKRGYGETWDPPPKPLPASRSGEGPSMADKKKADDDNNNSNMDDELLAVLGYKVRSSEMAEVAQKLEQLEMVLSNDDVGSTVLNDSVHYNPSDLSNWVESMLSELNNPASSDLDTTRSCVDRSEYDLRAIPGLSAFPKEEEVFDEEASSKRIRLGSWCESSDESTRSVVLVDSQETGVRLVHALVACAEAIHQENLNLADALVKRVGTLAGSQAGAMGKVATYFAQALARRIYRDYTAETDVCAAVNPSFEEVLEMHFYESCPYLKFAHFTANQAILEAVTTARRVHVIDLGLNQGMQWPALMQALALRPGGPPSFRLTGIGPPQTENSDSLQQLGWKLAQFAQNMGVEFEFKGLAAESLSDLEPEMFETRPESETLVVNSVFELHRLLARSGSIEKLLNTVKAIKPSIVTVVEQEANHNGIVFLDRFNEALHYYSSLFDSLEDSYSLPSQDRVMSEVYLGRQILNVVAAEGSDRVERHETAAQWRIRMKSAGFDPIHLGSSAFKQASMLLSLYATGDGYRVEENDGCLMIGWQTRPLITTSAWKLA

SEQ ID NO: 9 - DNA, 1740 bp, Arabidopsis thaliana
gtttttaatcactcaaccattaaaccccattttgatctctagttttttaaaagcaggagattttccttttcccagaaaagaaatttcccaaatcaaagtttcgagctttcacttctcgacttgcaaattctcgtcctttttactgaattcgatctgggtttttgtttttgattagtaaaataacaaaaaaaaaaaaaaggatttatcagaaatggctgcgagcgatgaagttaatcttattgagagcagaacagtggttcctctcaatacatgggttttaatatccaacttcaaagtagcctacaatatccttcgtcgccctgatggaacctttaaccgacacttagctgagtatctagaccgtaaagtcactgcaaacgccaatccggttgatggggttttctcgttcgatgtcttgattgatcgcaggatcaatcttctaagcagagtctatagaccagcttatgcagatcaagagcaacctcctagtattttagatctcgagaagcctgttgatggcgacattgtccctgttatattgttcttccatggaggtagctttgctcattcttctgcaaacagtgccatctacgatactctttgtcgcaggcttgttggtttgtgcaagtgtgttgttgtctctgtgaattatcggcgtgcaccagagaatccatacccttgtgcttatgatgatggttggattgctcttaattgggttaactcgagatcttggcttaaatccaagaaagactcaaaggtccatattttcttggctggtgatagctctggaggtaacatcgcgcataatgtggctttaagagcgggtgaatcgggaatcgatgttttggggaacattctgctgaatcctatgtttggtgggaatgagagaacggagtctgagaaaagtttggatgggaaatactttgtgacggttagagaccgcgattggtactggaaagcgtttttacccgagggagaagatagagagcatccagcgtgtaatccgtttagcccgagagggaaaagcttagaaggagtgagtttccccaagagtcttgtggttgtcgcgggtttggatttgattagagattggcagttggcatacgcggaagggctcaagaaagcgggtcaagaggttaagcttatgcatttagagaaagcaactgttgggttttacctcttgcctaataacaatcatttccataatgttatggatgagatttcggcgtttgtaaacgcggaatgttaacactgggttagagaaagaaggttgttttaacaaagccaagacatctttcaaactaacacacaggtgaatgtattgcctgtggattctctcgtttagttttgtttttgtgtttagtatctaagtgtgtggcggtctgcggcagcctttgtgatgactgtttaaacgctggattctgaaacgctaaagcttgtggaagaacagtgaggcgtttagagacttggaaaggaaccaagcactagtaaaaatttctcctttttttgtctgtaatatttggcatttagcttttacccttgagcctttttactaactaaaagctgattttttcagcatgagagtggtaattagatatctataaatatatatatttcaagaatgtaatgtttatacacaaattttagtgattttggtaaatgtatgtagggtctgcactctgcagttgtattgttgctcctctttttcattgtactctaatggattttacaaaaataagc

SEQ ID NO: 10 - protein, 345 aa, Arabidopsis thaliana
MAASDEVNLIESRTVVPLNTWVLISNFKVAYNILRRPDGTFNRHLAEYLDRKVTANANPVDGVFSFDVLIDRRINLLSRVYRPAYADQEQPPSILDLEKPVDGDIVPVILFFHGGSFAHSSANSAIYDTLCRRLVGLCKCVVVSVNYRRAPENPYPCAYDDGWIALNWVNSRSWLKSKKDSKVHIFLAGDSSGGNIAHNVALRAGESGIDVLGNILLNPMFGGNERTESEKSLDGKYFVTVRDRDWYWKAFLPEGEDREHPACNPFSPRGKSLEGVSFPKSLVVVAGLDLIRDWQLAYAEGLKKAGQEVKLMHLEKATVGFYLLPNNNHFHNVMDEISAFVNAEC

SEQ ID NO: 11 - DNA, 2015 bp, Arabidopsis thaliana
acatgcgaaattataatggcctgcctctcttccttcttatctcttttacttacactctccaggtccctcacttccctcattggacctctctaactctcctctcttaccttctcctgtttaaattcttctcctttctttccacaatttctgtctaaccaattccaacaccaaaaaattccatttcttgacgatgaaacgaagccatcaagaaacgtctgtagaagaagaagctccttcaatggtggagaagttagaaaatggttgtggtggtggtggagacgataacatggacgagtttcttgctgttttgggttacaaggttcgatcttcagacatggcagatgttgcacagaagcttgaacagcttgaaatggtcttgtccaatgatattgcctcttctagtaatgccttcaatgacaccgttcattacaatccttctgatctctccggttgggctcagagcatgctctcggatcttaattactacccggatcttgacccgaaccggatttgcgatctgagaccaatcacagacgacgatgagtgttgcagtagcaatagtaacagcaacaagaggattcgactcggtccttggtgtgactcagtgaccagcgagtcaactcgttccgtggtgcttatcgaggagacaggagttagactcgttcaggcgctagtggcctgcgccgaggcggttcagctggagaatctgagcctcgcggatgctctcgtcaagcgcgtgggattactcgcggcttctcaagccggagccatggggaaagtcgctacctacttcgccgaagccctagctcgtcgaatttaccggattcatccttccgccgccgccattgatccttccttcgaagagattcttcagatgaacttctacgactcgtgtccctacctgaaattcgctcatttcacggccaatcaggcgattctagaagctgttacgacgtcgcgtgtcgtacacgtaatcgatctagggcttaatcaaggtatgcaatggccggcgttaatgcaagccttagctctccgacccggtggtccaccgtcgtttcgtctcaccggcgttgggaatccgtcgaatcgagaagggattcaagagttaggttggaagctagctcagctggctcaagccatcggcgtcgaattcaaattcaatggtctaacgacggagaggttatccgatttagaaccggatatgttcgagacccgaaccgaatcggagactctagtggttaattcggttttcgagcttcacccggttttatcccaacccggttcgatcgaaaagctgttagcgacggttaaggcggttaaaccgggtctcgtaacagtggtggaacaagaagcgaaccataacggtgacgttttcttagaccggtttaacgaagcgcttcactattactcgagcttgttcgactcgctcgaagatggtgttgtgataccgagtcaagaccgagtcatgtcggaggtttacttagggagacagatattgaacttggtggcgacggaaggaagcgataggatcgagcgacacgagacgctggctcagtggcgaaaacgtatgggatccgccgggtttgacccggttaacctcggatcagacgcgtttaagcaagcgagtttgctattggcgttatctggcggtggagatggatacagagtggaggagaacgacggaagcctaatgcttgcgtggcaaacgaaacctctaatcgctgcatcggcgtggaaactagcggcggagttgcggcggtagatacgtcgtcataaagaggagaagaaaaaagacttagcgaacgtgaccttatgtttttattttactttaacttaccccagtagtttcgttttgtgacaatttcgcccgaaatattccgtgccttatacttttgggacccagttggttcgttggtcgtggagattcgagaacgaggaacatgtgtgtatgtaacaacagcacgagcaagtgttttcatagtttgaataaatatgaaagaaatgacgtttatttt

SEQ ID NO: 12 - protein, 523 aa, Arabidopsis thaliana
MKRSHQETSVEEEAPSMVEKLENGCGGGGDDNMDEFLAVLGYKVRSSDMADVAQKLEQLEMVLSNDIASSSNAFNDTVHYNPSDLSGWAQSMLSDLNYYPDLDPNRICDLRPITDDDECCSSNSNSNKRIRLGPWCDSVTSESTRSVVLIEETGVRLVQALVACAEAVQLENLSLADALVKRVGLLAASQAGAMGKVATYFAEALARRIYRIHPSAAAIDPSFEEILQMNFYDSCPYLKFAHFTANQAILEAVTTSRVVHVIDLGLNQGMQWPALMQALALRPGGPPSFRLTGVGNPSNREGIQELGWKLAQLAQAIGVEFKFNGLTTERLSDLEPDMFETRTESETLVVNSVFELHPVLSQPGSIEKLLATVKAVKPGLVTVVEQEANHNGDVFLDRFNEALHYYSSLFDSLEDGVVIPSQDRVMSEVYLGRQILNLVATEGSDRIERHETLAQWRKRMGSAGFDPVNLGSDAFKQASLLLALSGGGDGYRVEENDGSLMLAWQTKPLIAASAWKLAAELRR

SEQ ID NO: 13 - DNA, 3261 bp, Arabidopsis thaliana
atgaaagctccatcaaatggatttcttccaagttccaacgaaggagagaagaagccaatcaattctcaactatggcacgcttgtgcagggcctttagtttcattacctcctgtgggaagtcttgtggtttacttccctcaaggacacagcgagcaagttgcagcatcgatgcagaagcaaacagattttataccaaattacccaaatcttccttctaagctgatttgcttgcttcacagtgttacattacatgctgataccgaaacagatgaagtctatgcacaaatgactcttcaacctgtgaataagtatgatagagaagcattgctagcttctgatatgggcttgaagctaaacagacaacctactgagtttttttgcaagactcttactgcaagtgacacaagcactcatggtggattctctgtaccgcgtcgtgcagctgagaaaatattccctcctcttgatttctcgatgcaaccgcctgcgcaagagattgtagctaaagatttacatgatactacatggactttcagacatatctatcgaggccaaccaaaaagacacttgcttaccacaggttggagcgtttttgttagcacaaagagactatttgcgggtgattcagttttgtttgtaagagatgagaaatcacagctgatgttgggtataagacgtgcaaatagacaaactccgactctttcctcatcggtcatatccagcgacagtatgcacattgggatacttgcagctgcagctcatgctaatgccaatagtagcccttttaccatcttcttcaatccaagggcaagtccttcagagtttgtagttcctttagccaaatacaacaaagccttatacgctcaagtatctctaggaatgagattccggatgatgtttgagactgaggattgtggggttcgtagatatatgggtacagtcacaggtattagtgatcttgaccctgtaagatggaaaggctcacaatggcgtaatcttcaggtaggatgggatgaatcaacagctggagataggccaagccgagtatccatatgggaaatcgaacccgtcataactcctttttacatatgtcctcctccatttttcagacctaagtacccgaggcaacccgggatgccagatgatgagttagacatggaaaatgctttcaaaagagcaatgccttggatgggagaagactttgggatgaaggacgcacagagttcgatgttccctggtttaagtctagttcaatggatgagtatgcagcaaaacaatccattgtcaggttctgctactcctcagctcccgtccgcgctctcatcttttaacctaccaaacaattttgcttccaacgacccttccaagctgttgaacttccaatccccaaacctctcttccgcaaattcccaattcaacaaaccgaacacggttaaccatatcagccaacagatgcaagcacaaccagccatggtgaaatctcaacaacaacaacaacaacaacaacaacaacaccaacaccaacaacaacaactgcaacaacaacaacaactacagatgtcacagcaacaggtgcagcaacaagggatttataacaatggtacgattgctgttgctaaccaagtctcttgtcaaagtccaaaccaacctactggattctctcagtctcagcttcagcagcagtcaatgctccctactggtgctaaaatgacacaccagaacataaattctatggggaataaaggcttgtctcaaatgacatcgtttgcgcaagaaatgcagtttcagcagcaactggaaatgcataacagtagccagttattaagaaaccagcaagaacagtcctctctccattcattacaacaaaatctgtcccaaaatcctcagcaactccaaatgcaacaacaatcatcaaaaccaagtccttcacaacagcttcagttgcagctactgcagaagctacagcagcagcaacagcagcagtcgattcctccagtaagctcatccttacagccacaattatcagcgttgcagcagacacaaagccatcaattgcaacaacttctgtcgtctcaaaatcaacagcccttggcacatggtaataacagcttcccagcttcaactttcatgcagcctccacagattcaggtgagtcctcagcagcaaggacagatgagtaacaaaaatcttgtagccgctggaagatcacattctggccacacagatggagaagctccttcttgttcaacctcaccttccgccaataacacgggacatgataatgtttcaccgacaaatttcctgagcagaaatcaacagcaaggacaagctgcatctgtatctgcatctgattcagtctttgagcgcgcaagcaatccggtccaagagctttatacaaaaactgagagccggatcagtcaaggcatgatgaatatgaagagtgctggtgaacatttcagatttaaaagcgcggtaacagatcaaatcgatgtatccacagcgggaacgacgtactgtcctgatgttgttggccctgtacagcagcaacaaactttcccactaccatcatttggttttgatggagactgccaatctcatcatccaagaaacaacttagctttccctggtaatctcgaagccgtaacttctgatccactctattctcaaaaggactttcaaaacttggttcccaactatggcaacacaccaagagacattgagacggagctgtccagtgctgcaatcagttctcagtcatttggtattcccagcattccctttaagcccggatgttcaaatgaggttggcggcatcaatgattcaggaatcatgaatggtggaggactgtggcccaatcagactcaacgaatgcgaacatatacaaaggttcaaaaacgagggtcagtaggtagatcaatagatgttacccgttatagcggctatgatgaacttaggcatgacttagcgagaatgtttggcatcgaaggacagctcgaagatccgctaacctctgattggaaactcgtctacaccgatcacgaaaacgatattttactagttggtgatgatccttgggaagagtttgtgaactgcgtgcagaacataaagatactatcatcagtagaagttcagcaaatgagcttagacggagatcttgcagctatcccaaccacaaaccaagcctgcagcgaaacagacagcggaaatgcttggaaagtacactatgaagacacttctgctgcagcttctttcaacagatag

SEQ ID NO: 14 - protein, 1086 aa, Arabidopsis thaliana
MKAPSNGFLPSSNEGEKKPINSQLWHACAGPLVSLPPVGSLVVYFPQGHSEQVAASMQKQTDFIPNYPNLPSKLICLLHSVTLHADTETDEVYAQMTLQPVNKYDREALLASDMGLKLNRQPTEFFCKTLTASDTSTHGGFSVPRRAAEKIFPPLDFSMQPPAQEIVAKDLHDTTWTFRHIYRGQPKRHLLTTGWSVFVSTKRLFAGDSVLFVRDEKSQLMLGIRRANRQTPTLSSSVISSDSMHIGILAAAAHANANSSPFTIFFNPRASPSEFVVPLAKYNKALYAQVSLGMRFRMMFETEDCGVRRYMGTVTGISDLDPVRWKGSQWRNLQVGWDESTAGDRPSRVSIWEIEPVITPFYICPPPFFRPKYPRQPGMPDDELDMENAFKRAMPWMGEDFGMKDAQSSMFPGLSLVQWMSMQQNNPLSGSATPQLPSALSSFNLPNNFASNDPSKLLNFQSPNLSSANSQFNKPNTVNHISQQMQAQPAMVKSQQQQQQQQQQHQHQQQQLQQQQQLQMSQQQVQQQGIYNNGTIAVANQVSCQSPNQPTGFSQSQLQQQSMLPTGAKMTHQNINSMGNKGLSQMTSFAQEMQFQQQLEMHNSSQLLRNQQEQSSLHSLQQNLSQNPQQLQMQQQSSKPSPSQQLQLQLLQKLQQQQQQQSIPPVSSSLQPQLSALQQTQSHQLQQLLSSQNQQPLAHGNNSFPASTFMQPPQIQVSPQQQGQMSNKNLVAAGRSHSGHTDGEAPSCSTSPSANNTGHDNVSPTNFLSRNQQQGQAASVSASDSVFERASNPVQELYTKTESRISQGMMNMKSAGEHFRFKSAVTDQIDVSTAGTTYCPDVVGPVQQQQTFPLPSFGFDGDCQSHHPRNNLAFPGNLEAVTSDPLYSQKDFQNLVPNYGNTPRDIETELSSAAISSQSFGIPSIPFKPGCSNEVGGINDSGIMNGGGLWPNQTQRMRTYTKVQKRGSVGRSIDVTRYSGYDELRHDLARMFGIEGQLEDPLTSDWKLVYTDHENDILLVGDDPWEEFVNCVQNIKILSSVEVQQMSLDGDLAAIPTTNQACSETDSGNAWKVHYEDTSAAASFNR

SEQ ID NO: 15 - DNA, 3497 bp, Arabidopsis thaliana
tgaaagctccttcatcaaatggagtttctcctaatcctgttgaaggagaaaggagaaatataaactcagagctatggcacgcttgtgctgggccattgatttcgttgcctccagcaggaagtcttgttgtttacttccctcaaggtcacagtgagcaagtcgcggcttcaatgcagaagcagactgatttcataccaagttacccgaatcttccttccaagctcatatgcatgctccacaatgttacactgaatgctgatcctgagacggatgaggtctatgcgcagatgactcttcagccagtaaacaaatatgacagagatgcattgcttgcttctgacatgggtcttaagctaaacagacaacctaatgaatttttctgcaaaaccctcacggcgagtgacacaagtactcacggtggattttctgtaccccgacgagctgctgagaaaatctttcctgctctggatttctcgatgcaaccaccttgtcaggagcttgttgctaaggatattcatgacaacacatggactttcagacatatttatcgaggtcaaccaaaaaggcacttgctaactacaggctggagtgtgtttgtcagcacgaaaaggctctttgctggagactctgttctttttataagagatggaaaggcgcaacttctgttggggataagacgtgcaaatagacaacagcctgcactttcttcatctgtaatatcaagtgatagcatgcacatcggagttcttgcagctgcagctcatgctaatgctaataacagtcctttcaccattttctacaacccgaggtgggctgctcctgctgagtttgtggttcctttagccaagtataccaaagcgatgtacgctcaagtttccctcggtatgcggtttagaatgatatttgagactgaagaatgtggagttcgtcggtatatgggtacagttaccggtatcagtgatcttgatccagtgagatggaaaaactctcagtggcggaatcttcagattggatgggatgagtcagctgctggtgataggcccagtcgagtttcagtttgggacattgaaccggttttaactcctttctacatatgtcctcctccatttttccgacctcgcttttctggacaacctggaatgccagatgatgagactgacatggagtctgcactgaagagagcaatgccatggcttgataatagcttagagatgaaagacccttcgagtactatctttcctggtctgagtttagttcagtggatgaatatgcagcagcagaacggccagctaccctctgctgctgcacagccaggtttcttcccatcaatgctttcgccaaccgcggcgctgcacaacaatcttggcggcactgatgatccctccaagttactgagctttcagacgccgcacggggggatttcctcctcaaatctccaatttaacaaacagaatcagcaagccccaatgtctcagttgcctcagccaccaactacgttgtcccaacaacagcagctgcagcaattgttgcactcctctttgaaccatcaacaacagcaatcgcagtctcaacaacagcaacaacaacaacagttgctgcagcagcaacaacaattgcagtctcaacaacacagcaacaacaatcaatcgcagtctcagcaacaacaacaattgctccagcagcaacaacaacaacaactgcagcaacaacatcaacaaccgttacagcaacagactcagcagcagcagctaagaacacagccattgcaatctcactcgcatccacagccacaacagttacaacaacataagttgcagcaacttcaggttccacagaatcagctttacaatggtcaacaagcagcgcagcagcatcagtcgcaacaagcatctacacatcatttgcaaccacaattagtttcgggatcaatggcaagcagtgtcatcacgcctccgtccagctcccttaatcaaagctttcaacagcaacaacaacagtctaagcaacttcaacaagcacatcaccatttaggtgctagcactagccagagtagtgtaattgaaaccagcaagtcttcatccaatctgatgtccgcaccgccgcaagagacacagttttcacgacaagtagaacagcagcagcctcctggtctcaacgggcagaatcagcaaacacttttgcagcagaaagctcaccaggcacaggcccaacagatattccagcagagtctcttggaacagccgcatatacagtttcagctgttacagagattacaacagcaacagcagcagcaatttctttcgccgcagtctcagttaccacaccatcaattgcaaagccagcagttgcaacagctgcctactctctctcaaggtcatcagtttccgtcatcttgcactaacaatggcttatcgacgttgcaaccacctcaaatgctggtgagccgacctcaggaaaaacaaaacccaccggttgggggaggggtcaaagcttattcaggcatcacagatggaggagatgcaccttcctcttcaacgtcgccttccaccaacaactgtcagatctcttcttcaggctttctcaacagaagccaaagcgggccagcgatcttgatacctgatgcagcgattgatatgtctggtaatcttgttcaggatctttacagcaaatccgatatgcggctaaaacaagaactcgtgggtcagcaaaagtccaaagctagtttaacagatcatcaactagaagcatctgcctctggaacttcttacggtttagatggaggcgaaaacaacagacaacaaaatttcttggctccaacttttggccttgacggtgattccaggaacagcttgctcggtggagctaatgttgataatggctttgtgcctgacacgctactctcgaggggatatgactcccagaaagatcttcagaacatgctttcaaactatggaggagtgacaaatgacattggtacagagatgtctacttcagctgtaagaactcaatcttttggtgtccccaatgtgcccgccatttcgaacgatctagctgtcaacgatgctggagttcttggtggtggattgtggccagctcagactcagcgaatgcgaacttatacaaaggtgcaaaaacgaggctcagtggggagatcaatagacgtcaaccgttacagaggttacgatgagctgaggcatgatctagcgcgcatgtttgggatcgaaggacagctcgaagatcctcaaacatctgactggaaacttgtttatgtcgatcatgaaaatgacatcctcctcgtcggcgatgatccatgggaagaattcgtaaactgtgttcagagcattaagatcctttcatcagctgaggttcagcagatgagcttagacgggaactttgccggtgtaccagttactaatcaagcttgtagtggcggtgacagtggcaatgcttggagaggtcattatgatgataactcagccacttcgtttaaccggtga

SEQ ID NO: 16 - protein, 1164 aa, Arabidopsis thaliana
MKAPSSNGVSPNPVEGERRNINSELWHACAGPLISLPPAGSLVVYFPQGHSEQVAASMQKQTDFIPSYPNLPSKLICMLHNVTLNADPETDEVYAQMTLQPVNKYDRDALLASDMGLKLNRQPNEFFCKTLTASDTSTHGGFSVPRRAAEKIFPALDFSMQPPCQELVAKDIHDNTWTFRHIYRGQPKRHLLTTGWSVFVSTKRLFAGDSVLFIRDGKAQLLLGIRRANRQQPALSSSVISSDSMHIGVLAAAAHANANNSPFTIFYNPRAAPAEFVVPLAKYTKAMYAQVSLGMRFRMIFETEECGVRRYMGTVTGISDLDPVRWKNSQWRNLQIGWDESAAGDRPSRVSVWDIEPVLTPFYICPPPFFRPRFSGQPGMPDDETDMESALKRAMPWLDNSLEMKDPSSTIFPGLSLVQWMNMQQQNGQLPSAAAQPGFFPSMLSPTAALHNNLGGTDDPSKLLSFQTPHGGISSSNLQFNKQNQQAPMSQLPQPPTTLSQQQQLQQLLHSSLNHQQQQSQSQQQQQQQQLLQQQQQLQSQQHSNNNQSQSQQQQQLLQQQQQQQLQQQHQQPLQQQTQQQQLRTQPLQSHSHPQPQQLQQHKLQQLQVPQNQLYNGQQAAQQHQSQQASTHHLQPQLVSGSMASSVITPPSSSLNQSFQQQQQQSKQLQQAHHHLGASTSQSSVIETSKSSSNLMSAPPQETQFSRQVEQQQPPGLNGQNQQTLLQQKAHQAQAQQIFQQSLLEQPHIQFQLLQRLQQQQQQQFLSPQSQLPHHQLQSQQLQQLPTLSQGHQFPSSCTNNGLSTLQPPQMLVSRPQEKQNPPVGGGVKAYSGITDGGDAPSSSTSPSTNNCQISSSGFLNRSQSGPAILIPDAAIDMSGNLVQDLYSKSDMRLKQELVGQQKSKASLTDHQLEASASGTSYGLDGGENNRQQNFLAPTFGLDGDSRNSLLGGANVDNGFVPDTLLSRGYDSQKDLQNMLSNYGGVTNDIGTEMSTSAVRTQSFGVPNVPAISNDLAVNDAGVLGGGLWPAQTQRMRTYTKVQKRGSVGRSIDVNRYRGYDELRHDLARMFGIEGQLEDPQTSDWKLVYVDHENDILLVGDDPWEEFVNCVQSIKILSSAEVQQMSLDGNFAGVPVTNQACSGGDSGNAWRGHYDDNSATSFNR

SEQ ID NO: 17 - DNA, 3168 bp, Oryza sativa
atgaaggatcagggatcatccggtgtgtctcccgccccaggggaaggggagaagaaagccatcaattcggagctatggcatgcttgtgccgggcctcttgtgtcgctgccgccggtgggcagtctcgtcgtgtacttccctcagggtcatagcgagcaggttgctgcttccatgcacaaggagctggacaacatccctggttatccctctcttccgtctaagctgatctgcaaacttctgagtctcaccttacatgcagattctgaaactgatgaagtttatgctcagatgacacttcaaccagtcaataaatatgatcgagatgcaatgctggcatctgaactgggcctgaagcaaaacaagcaaccagcggagttcttttgcaaaacgctgacggcgagcgacacaagtacccatggtggattttcagtgccacgtcgtgcggcggagaagatatttccaccactagactttaccatgcaaccaccagcacaagagctcatcgccaaggatctgcatgatatttcatggaaatttcgacacatttaccgaggtcaaccaaagaggcaccttctgacaactggttggagcgtctttgtcagcacaaaaaggcttctagctggtgattcagttctgtttataagggatgagaaatctcagcttctattaggcatacgtcgtgctaccagaccccaaccagctctatcgtcatcagttctatcaagtgatagcatgcacattgggattctagctgctgcagcacatgctgctgcaaacagtagcccatttactattttctacaatccaagggcaagtccatcagaatttgtcattcctttagcgaaatataacaaggctttgtatacacaagtatctcttggaatgcggttcagaatgctgtttgagacagaggattcaggggttcgaagatatatgggaacaatcacaggtattggtgacttggatccagtgcgctggaagaactctcattggcgaaaccttcaggttggttgggatgaatcaacagcatctgagaggcgcactcgtgtttcaatatgggagattgaaccagtcgcgacacctttttatatttgtccaccaccatttttcaggccaaaacttcctaagcagccaggaatgccagatgatgaaaatgaagttgagagtgctttcaaaagagccatgccatggcttgctgatgactttgccctgaaagatgtgcaaagtgcattatttccaggtctgagcctagtccaatggatggctatgcaacagaatcctcagatgctaacagctgcgtcccaaacagtgcaatcaccgtacttgaactccaatgcattggctatgcaggatgtgatgggtagtagcaacgaggacccaacaaaaagattgaacacacaggcacaaaatatggttttacctaatttacaggttggctcaaaagtggatcaccctgtaatgtctcaacatcaacagcagccacaccaactatcacaacagcagcaggtccagccatcgcagcaaagttctgtggttttacagcaacatcaagcccagttgctgcagcagaacgccattcacttgcagcagcagcaagaacatctccagcggcagcagtcacaaccggcacagcagttgaaggctgcttcaagtctgcattcagtggaacagcacaagctgaaagaacagacttcaggtgggcaggttgcctcacaagcacaaatgttaaaccagattttcccaccatcttcatcgcagctacaacagttaggtttacccaagtcacctactcatcgccaagggttgacaggattaccaattgcaggttctttgcagcagcccacactaactcagacatctcaagtccagcaagcagccgaatatcagcaggccctcctacagagtcagcaacagcaacagcaactgcaactgcaacaactatcacaaccagaagtacagctgcagctgcttcaaaagattcaacaacaaaacatgctatctcagctgaacccacaacatcagtcccagttgattcaacaattgtctcagaaaagccaggaaattctacagcaacaaattttgcaacatcaatttggtgggtctgattctattggtcaactcaagcaatcaccatcgcagcaagctcctttaaaccacatgacaggatctttgacgccccagcaacttgtcagatcacattcggcacttgctgagagtggggatccatccagttcaactgctccatccaccagccgtatttctccaataaattcgctgagtagggcaaaccaaggaagcagaaatttaactgacatggtggcaacaccacaaattgacaacttacttcaggaaattcaaagcaagccagataatcgaattaagaatgacatacagagcaaagaaacagtccctatacataaccgacatccagtttctgatcaacttgatgcatcatctgctacctccttttgtttagacgagagcccacgagaaggtttttccttccctccagtttgtttggataacaatgttcaagttgatccaagagataactttcttattgcggaaaatgtggacgcattgatgccagatgccctgttgtcaagaggtatggcttcaggaaagggcatgtgcactctgacttctggacaaagggatcacagggatgtcgagaatgagctatcatctgctgcattcagttcccagtcatttggtgtgcctgacatgtcctttaagcctggatgttcaagtgacgttgctgttactgatgccggaatgccaagccaaggtttgtggaataatcaaacacaacggatgagaactttcactaaggttcaaaagcgtggttctgtggggagatcaattgatatcacaagatatcgagattatgatgaacttaggcatgatcttgcatgcatgtttggtatccaaggtcaacttgaagatccatataggatggattggaagctagtctatgttgatcatgagaatgatatccttcttgtcggcgacgacccttgggaggaatttgtgggctgtgtgaagagcatcaaaatactctcagctgctgaagtacaacagatgagcttggatggtgaccttggtggcgtccctccacaaacacaggcctgtagtgcctctgatgatgcaaatgcatggagaggttga

SEQ ID NO: 18 - protein, 1053 aa, Oryza sativa
MKDQGSSGVSPAPGEGEKKAINSELWHACAGPLVSLPPVGSLVVYFPQGHSEQVAASMHKELDNIPGYPSLPSKLICKLLSLTLHADSETDEVYAQMTLQPVNKYDRDAMLASELGLKQNKQPAEFFCKTLTASDTSTHGGFSVPRRAAEKIFPPLDFTMQPPAQELIAKDLHDISWKFRHIYRGQPKRHLLTTGWSVFVSTKRLLAGDSVLFIRDESQLLLGIRRATRPQPALSSSVLSSDSMHIGILAAAAHAAANSSPFTIFYNPRASPSEFVIPLAKYNKALYTQVSLGMRFRMLFETEDSGVRRYMGTITGIGDLDPVRWKNSHWRNLQVGWDESTASERRTRVSIWEIEPVATPFYICPPPFFRPKLPKQPGMPDDENEVESAFKRAMPWLADDFALKDVQSALFPGLSLVQWMAMQQNPQMLTAASQTVQSPYLNSNALAMQDVMGSNEDPTKRLNTQAQNMVLPNLQVGSKVDHPVMSQHQQQPHQLSQQQQVQPSQQSSVVLQQHQAQLLQQNAIHLQQQQEHLQRQQSQPAQQLKAASSLHSVEQHKLKEQTSGGQVASQAQMLNQIFPPSSSQLQQLGLPKSPTHRQGLTGLPIAGSLQQPTLTQTSQVQQAAEYQQALLQSQQQQQQLQLQQLSQPEVQLQLLQKIQQQNMLSQLNPQHQSQLIQQLSQKSQEILQQQILQHQFGGSDSIGQLKQSPSQQAPLNHMTGSLTPQQLVRSHSALAESGDPSSSTAPSTSRISPINSLSRANQGSRNLTDMVATPQIDNLLQEIQSKPDNRIKNDIQSKETVPIHNRHPVSDQLDASSATSFCLDESPREGFSFPPVCLDNNVQVDPRDNFLIAENVDALMPDALLSRGMASGKGMCTLTSGQRDHRDVENELSSAAFSSQSFGVPDMSFKPGCSSDVAVTDAGMPSQGLWNNQTQRMRTFTKVQKRGSVGRSIDITRYRDYDELRHDLACMFGIQGQLEDPYRMDWKLVYVDHENDILLVGDDPWEEFVGCVKSIKILSAAEVQQMSLDGDLGGVPPQTQACSASDDANAWRG

SEQ ID NO: 19 - DNA, 3417 bp, Oryza sativa
atgatgaagcaggcgcagcagcagccgccgccgccaccggcgagctctgcggcgacgacgaccaccgcgatggcagccgctgcggcggcggcggtggtggggagcgggtgcgaaggggagaagacgaaggcgccggcgatcaactcggagctgtggcacgcctgcgcggggccgctggtgtcgctgccgccggcgggcagcctcgtcgtctacttcccccagggccacagcgagcaggcggacccagaaacagatgaagtgtatgcacaaatgactcttcagccagttacttcatatgggaaggaggccctgcagttatcagagcttgcactcaaacaagcgagaccacagacagaattcttttgcaagacactgactgcaagtgatacaagtactcatggaggcttctctgtgcctcgtcgagctgcagaaaagatatttcctccactggacttctcaatgcaaccacctgcacaagaactacaggccagggatttgcatgataatgtgtggacattccgtcacatatatcggggtcagccaaaaaggcatctgcttaccactggctggagtctatttgtaagcggcaagaggttatttgctggagattctgtcatttttgtcagggatgaaaagcagcaacttctattaggaatcaggcgtgctaaccgacagccaactaacatatcatcatctgtcctttcaagtgacagcatgcacatagggattcttgctgctgcagcccatgctgctgccaacaatagcccatttaccatcttttataaccctagggccagtcctactgaatttgttatcccatttgctaagtatcagaaggcagtctatggtaatcaaatatctttagggatgcgctttcgcatgatgtttgagactgaggaattaggaacacgaagatacatgggaacaataactggcataagtgatctagatccagtaagatggaaaaactcgcagtggcgcaacttacaggttggttgggatgaatccgcagccggtgaaaggcgaaatagggtttctatctgggagattgaaccggtcgctgctccatttttcatatgtcctccaccattttttggtgcgaagcggcccaggcaattagatgacgagtcctcggaaatggagaatctcttaaagagggctatgccttggcttggtgaggaaatatgcataaaggatcctcagactcagaacaccataatgcctgggctgagcttggttcagtggatgaacatgaacatgcaacagagctcctcatttgcgaatacagccatgcagtctgagtaccttcgatcattgagcaaccccaacatgcaaaatcttggtgccgccgatctctctaggcaattatgcctgcagaaccagcttcttcaacagaacaatatacagtttaatactcccaaactttctcagcaaatgcagccagtcaatgagttagcaaaggcaggcattccgttgaatcagcttggtgtgagcaccaaacctcaggaacagattcatgatgctagcaaccttcagaggcaacaaccttccatgaaccatatgcttcctttgagccaagctcaaaccaatcttggccaagctcaggtccttgtccaaaatcaaatgcaacagcaacatgcatcttcaactcaaggtcaacaaccagctaccagccagcccttgcttctgccccagcagcagcaacagcagcagcagcagcagcaacaacaacaacaacagcaacaacaacaaaaattgctacaacagcagcagcaacagcttttgctccagcaacagcagcaattgagtaagatgcctgcacagttgtcaagtctggcgaatcagcagtttcagctaactgatcaacagcttcagctgcaactgttacaaaaactacagcaacaacagcagtcattgctttcacaacctgcagtcacccttgcacaattacctctgatccaagaacagcagaagttacttctggatatgcaacagcagctgtcaaactcccaaacactttcccaacaacaaatgatgcctcaacaaagtaccaaggttccatcacagaacacaccattgccactgcctgtgcaacaagagccacaacagaagcttctacagaagcaagcgatgctagcagacacttcagaagctgccgttccgccgaccacatcagtcaatgtcatttcaacaactggaagccctttgatgacaactggtgctactcattctgtacttacagaagaaatcccttcttgttcaacatcaccatccacagctaatggcaatcaccttctacaaccaatacttggtaggaacaaacattgtagcatgatcaacacagaaaaggttcctcagtctgctgctcctatgtcagttccaagctcccttgaagctgtcacagcaaccccgagaatgatgaaggattcaccaaagttgaaccataatgttaaacaaagtgtagtggcttcaaaattagcaaatgctgggactggttctcaaaattatgtgaacaatccacctccaacggactatctggaaactgcttcttccgcaacttcagtgtggctttcccagaatgatggacttctacatcaaaatttccctatgtccaacttcaaccagccacagatgttcaaagatgctcctcctgatgctgaaattcatgctgctaatacaagtaacaatgcattgtttggaatcaatggtgatggtccgctgggcttccctataggactaggaacagatgatttcctgtcgaatggaattgatgctgccaagtacgagaaccatatctcaacagaaattgataatagctacagaattccgaaggatgcccagcaagaaatatcatcctcaatggtttcacagtcatttggtgcatcagatatggcatttaattcaattgattccacgatcaacgatggtggctttttgaaccggagttcttggcctcctgccgctcccttaaagaggatgaggacattcaccaaggtatataagcgaggagctgtaggccggtccattgacatgagtcagttctctggatatgatgaattaaagcatgctctggcacggatgttcagtatagaggggcaacttgaggaacggcagagaattggttggaagctcgtttacaaggatcatgaagatgacatcctacttcttggcgacgacccatgggaggaatttgtcggttgcgtgaaatgcattaggatcctttcacctcaagaagttcagcagatgagcttggagggttgtgatctcgggaacaacattcccccgaatcaggcctgcagcagctcagacggagggaatgcatggagggctcgctgcgatcagaactccgaggccattcttaagatctccatgatgaaatcaaaagttgaagatgtcaggtattggaatactgcgtaa

SEQ ID NO: 20 - protein, 1138 aa, Oryza sativa
MMKQAQQQPPPPPASSAATTTTAMAAAAAAAVVGSGCEGEKTKAPAINSELWHACAGPLVSLPPAGSLVVYFPQGHSEQADPETDEVYAQMTLQPVTSYGKEALQLSELALKQARPQTEFFCKTLTASDTSTHGGFSVPRRAAEKIFPPLDFSMQPPAQELQARDLHDNVWTFRHIYRGQPKRHLLTTGWSLFVSGKRLFAGDSVIFVRDEKQQLLLGIRRANRQPTNISSSVLSSDSMHIGILAAAAHAAANNSPFTIFYNPRASPTEFVIPFAKYQKAVYGNQISLGMRFRMMFETEELGTRRYMGTITGISDLDPVRWKNSQWRNLQVGWDESAAGERRNRVSIWEIEPVAAPFFICPPPFFGAKRPRQLDDESSEMENLLKRAMPWLGEEICIKDPQTQNTIMPGLSLVQWMNMNMQQSSSFANTAMQSEYLRSLSNPNMQNLGAADLSRQLCLQNQLLQQNNIQFNTPKLSQQMQPVNELAKAGIPLNQLGVSTKPQEQIHDASNLQRQQPSMNHMLPLSQAQTNLGQAQVLVQNQMQQQHASSTQGQQPATSQPLLLPQQQQQQQQQQQQQQQQQQQQKLLQQQQQQLLLQQQQQLSKMPAQLSSLANQQFQLTDQQLQLQLLQKLQQQQQSLLSQPAVTLAQLPLIQEQQKLLLDMQQQLSNSQTLSQQQMMPQQSTKVPSQNTPLPLPVQQEPQQKLLQKQAMLADTSEAAVPPTTSVNVISTTGSPLMTTGATHSVLTEEIPSCSTSPSTANGNHLLQPILGRNKHCSMINTEKVPQSAAPMSVPSSLEAVTATPRMMKDSPKLNHNVKQSVVASKLANAGTGSQNYVNNPPPTDYLETASSATSVWLSQNDGLLHQNFPMSNFNQPQMFKDAPPDAEIHAANTSNNALFGINGDGPLGFPIGLGTDDFLSNGIDAAKYENHISTEIDNSYRIPKDAQQEISSSMVSQSFGASDMAFNSIDSTINDGGFLNRSSWPPAAPLKRMRTFTKVYKRGAVGRSIDMSQFSGYDELKHALARMFSIEGQLEERQRIGWKLVYKDHEDDILLLGDDPWEEFVGCVKCIRILSPQEVQQMSLEGCDLGNNIPPNQACSSSDGGNAWRARCDQNSEAILKISMMKSKVEDVRYWNTA

SEQ ID NO: 21 - DNA, 25 bp, Artificial
  LC15 primer
ttaatctgtttggttacccttgcgg

SEQ ID NO: 22 - DNA, 24 bp, Artificial
  LC18 primer
gacagggatgcatattttgtgaag

SEQ ID NO: 23 - DNA, 20 bp, Artificial
  LC69 primer
ccgtcggagctttattcttg

SEQ ID NO: 24 - DNA, 20 bp, Artificial
  LC70 primer
tcgttcctatgactccacca

SEQ ID NO: 25 - DNA, 24 bp, Artificial
  LC73 primer
caccatggctgcgagcgatgaagt

SEQ ID NO: 26 - DNA, 21 bp, Artificial
  LC74 primer
acattccgcgtttacaaacgc

SEQ ID NO: 27 - DNA, 23 bp, Artificial
  LC75 primer
caccctagatccaagatcagacc

SEQ ID NO: 28 - DNA, 19 bp, Artificial
  LC76 primer
gtacgccgccgtcgagagt

SEQ ID NO: 29 - DNA, 37 bp, Artificial
  LC77 primer
gagatggcggaggttgctttgagactcgaacaattag

SEQ ID NO: 30 - DNA, 37 bp, Artificial
  LC78 primer
ctaattgttcgagtctcaaagcaacctccgccatctc

SEQ ID NO: 31 - DNA, 23 bp, Artificial
  LC80 primer
caccatgaagagagatcatcatc

SEQ ID NO: 32 - DNA, 20 bp, Artificial
  LC81 primer
attggtggagagtttccaag

SEQ ID NO: 33 - DNA, 20 bp, Artificial
  LC85 primer
gcctcaaaagacacctctgg

SEQ ID NO: 34 - DNA, 20 bp, Artificial
  LC86 primer
gcttatccagcttccacgtc

SEQ ID NO: 35 - DNA, 21 bp, Artificial
  lcm26 primer
ccgtcggagctttattcttgg

SEQ ID NO: 36 - DNA, 21 bp, Artificial
  lcm27 primer
cgtcgttcctatgactccacc

SEQ ID NO: 37 - DNA, 22 bp, Artificial
  lcm28 primer
gcaaaacctagatccgacattg

SEQ ID NO: 38 - DNA, 19 bp, Artificial
  lcm29 primer
gctccgccggattatagtg

SEQ ID NO: 39 - DNA, 23 bp, Artificial
  mr27 primer
ctctcccgctatgtatgtcgcca

SEQ ID NO: 40 - DNA, 20 bp, Artificial
  mr38 primer
gtgagacacaccatcaccag

SEQ ID NO: 41 - protein, 27 aa, Arabidopsis thaliana
DELLAVLGYKVRSSEMAEVALKLEQLE

SEQ ID NO: 42 - protein, 27 aa, Arabidopsis thaliana
DELLAVLGYKVRSSEMAEVAQKLEQLE

SEQ ID NO: 43 - protein, 27 aa, Arabidopsis thaliana
DELLAVLGYKVRSSEMAEVAHKLEQLE

SEQ ID NO: 44 - protein, 27 aa, Arabidopsis thaliana
DELLAVLGYKVRSSEMAEVAQKLEQLE

SEQ ID NO: 45 - protein, 27 aa, Arabidopsis thaliana
DELLAVLGYKVRSSEMAEVAQKLEQLE

SEQ ID NO: 46 - protein, 27 aa, Zea mays
DELLAVLGYKVRSSEMAEVAQKLEQLE

SEQ ID NO: 47 - protein, 27 aa, Oryza sativa
DELLAVLGYKVRSSEMAEVAQKLEQLE

SEQ ID NO: 48 - protein, 27 aa, Triticum aestivum
DELLAVLGYKVRSSEMAEVAQKLEQLE

SEQ ID NO: 49 - protein, 27 aa, Hordeum vulgare
DELLAVLGYKVRSSEMAEVAQKLEQLE

SEQ ID NO: 50 - protein, 30 aa, Arabidopsis thaliana
MAASDEVNLIESRTVVPLNTWVLISNFKVA

SEQ ID NO: 51 - protein, 30 aa, Arabidopsis thaliana
MAGGNEVNLNECKRIVPLNTWVLISNFKLA

SEQ ID NO: 52 - protein, 30 aa, Arabidopsis thaliana
MAGSEEVNLIESKTVVPLNTWVLISNFKLA

SEQ ID NO: 53 - protein, 30 aa, Oryza sativa
MAGSDEVNRNECKTVVPLHTWVLISNFKLS

SEQ ID NO: 54 - protein, 30 aa, Triticum aestivum
MAGSDEVNRNECKTVVPLHTWVLISNFKVS

SEQ ID NO: 55 - protein, 30 aa, Zea mays
MAGSDEVNRNECKGAVPIHTWVLISNFKLA

SEQ ID NO: 56 - protein, 35 aa, Arabidopsis thaliana
MAASDEVNLIESRTVVPLNTWVLISNFKVAYNILR

SEQ ID NO: 57 - protein, 13 aa, Arabidopsis thaliana
LIESRTVVPLNTW

SEQ ID NO: 58 - protein, 12 aa, Arabidopsis thaliana
VVPLNTWVLISN

SEQ ID NO: 59 - protein, 35 aa, Arabidopsis thaliana; identical to SEQ ID NO: 56

SEQ ID NO: 60 - protein, 13 aa, Arabidopsis thaliana
LIESRTVVPLNTW

SEQ ID NO: 61 - protein, 13 aa, Arabidopsis thaliana
VVPLNTWVLISNF

SEQ ID NO: 62 - protein, 587 aa, Arabidopsis thaliana; identical to SEQ ID NO: 4

SEQ ID NO: 63 - protein, 532 aa, Arabidopsis thaliana
MKRDHHHHHQDKKTMMMNEEDDGNGMDELLAVLGYKVRSSEMADVAQKLEQLEVMMSNVQEDDLSQLATETVHYNPAELYTWLDSMLTDLNPPSSNAEYDLKAIPGDAILNQFAIDSASSSNQGGGGDTYTTNKRLKCSNGVVETTTATAESTRHVVLVDSQENGVRLVHALLACAEAVQKENLTVAEALVKQIGFLAVSQIGAMRKVATYFAEALARRIYRLSPSQSPIDHSLSDTLQMHFYETCPYLKFAHFTANQAILEAFQGKKRVHVIDFSMSQGLQWPALMQALALRPGGPPVFRLTGIGPPAPDNFDYLHEVGCKLAHLAEAIHVEFEYRGFVANTLADLDASMLELRPSEIESVAVNSVFELHKLLGRPGAIDKVLGVVNQIKPEIFTVVEQESNHNSPIFLDRFTESLHYYSTLFDSLEGVPSGQDKVMSEVYLGKQICNVVACDGPDRVERHETLSQWRNRFGSAGFAAAHIGSNAFKQASMLLALFNGGEGYRVEESDGCLMLGWHTRPLIATSAWKLSTN

SEQ ID NO: 64 - protein, 625 aa, Oryza sativa
MKREYQEAGGSSGGGSSADMGSCKDKVMAGAAGEEEDVDELLAALGYKVRSSDMADVAQKLEQLEMAMGMGGVSAPGAADDGFVSHLATDTVHYNPSDLSSWVESMLSELNAPLPPIPPAPPAARHASTSSTVTGGGGSGFFELPAAADSSSSTYALRPISLPVVATADPSAADSARDTKRMRTGGGSTSSSSSSSSSLGGGASRGSVVEAAPPATQGAAAANAPAVPVVVVDTQEAGIRLVHALLACAEAVQQENFAAAEALVKQIPTLAASQGGAMRKVAAYFGEALARRVYRFRPADSTLLDAAFADLLHAHFYESCPYLKFAHFTANQAILEAFAGCHRVHVVDFGIKQGMQWPALLQALALRPGGPPSFRLTGVGPPQPDETDALQQVGWKLAQFAHTIRVDFQYRGLVAATLADLEPFMLQPEGEADANEEPEVIAVNSVFELHRLLAQPGALEKVLGTVHAVRPRIVTVVEQEANHNSGSFLDRFTESLHYYSTMFDSLEGGSSGQAELSPPAAGGGGGTDQVMSEVYLGRQICNVVACEGAERTERHETLGQWRNRLGRAGFEPVHLGSNAYKQASTLLALFAGGDGYRVEEKEGCLTLGWHTRPLIATSAWRVAAA

SEQ ID NO: 65 - protein, 555 aa, Triticum aestivum
MAMGMGGVGAGAAPDDSFATHLATDTVHYNPTDLSSWVESMLSELNAPPPPLPPAPQLNASTSSTVTGGGYFDLPPSVDSSCSTYALRPIPSPAVAPADLSADSVVRDPKRMRTGGSSTSSSSSSSSLGGGGARSSVVEAAPPVAAAAGAPALPVVVVDTQEAGIRLVHALLACAEAVQQENFSAAEALVKQIPLLAASQGGAMRKVAAYFGEALARRVFRFRPQPDSSLLDAAFADLLHAHFYESCPYLKFAHFTANQAILEAFAGCRRVHVVDFGIKQGMQWPALLQALALRPGGPPSFRLTGVGPPQPDETDALQQVGWKLAQFAHTIRVDFQYRGLVAATLADLEPFMLQPEGEENPNEEPEVIAVNSVFEMHRLLAQPGALEKVLGTVRAVRPRIVTVVEQEANHNSGTFLDRFTESLHYYSAMFDSLEGGSSGGPSEVSSGAAAAPAAAGTDQVMSEVYLGRQICNVVACEGAERTERHETLGQWRNRLGNAGFETVHLGSNAYKQASTLLALFAGGDGYKVEEKEGCLTLGWHTRPLIATSAWRLAAP

SEQ ID NO: 66 - protein, 30 aa, Arabidopsis thaliana
NLVQDLYSKSDMRLKQELVGQQKSKASLTD

SEQ ID NO: 67 - protein, 7 aa, Oryza sativa
NRIKNDI

SEQ ID NO: 68 - protein, 13 aa, Arabidopsis thaliana
QPAMVKSQQQQQQ

SEQ ID NO: 69 - protein, 16 aa, Oryza sativa
QPHQLSQQQQVQPSQQ

(19) United States
(12) Patent Application Publication
(10) Pub. No.: US 2015/0353950 A1
(43) Pub. Date: Dec. 10, 2015

(54) TRANSGENIC PLANTS

(71) Applicant: Durham University, Durham (GB)

(72) Inventor: Ari SADANANDOM, Durham (GB)

(21) Appl. No.: 14/723,638

(22) Filed: May 28, 2015

Related U.S. Application Data
(63) Continuation-in-part of application No. PCT/GB2013/051723, filed on Jun. 28, 2013.

(30) Foreign Application Priority Data
Nov. 29, 2012 (GB) 1221518.2
Mar. 28, 2013 (GB) 1305696.5

(51) Int. Cl. C12N 15/82; C07K 14/415

(52) U.S. Cl. CPC C12N 15/8271; C07K 14/415; C12N 15/8261; C12N 15/8273

(57) ABSTRACT

The invention relates to altering plant characteristics by manipulating plant genes.

### RELATED APPLICATIONS AND INCORPORATION BY REFERENCE

This application is a continuation-in-part application of international patent application Serial No. PCT/GB2013/051723 filed Jun. 28, 2013, which published as PCT Publication No. WO 2014/083301 on Jun. 5, 2014, which claims benefit of United Kingdom patent application Serial No. GB 1221518.2 filed Nov. 29, 2012 and United Kingdom patent application Serial No. GB 1305696.5 filed Mar. 28, 2013.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### SEQUENCE LISTING

The instant application contains a Sequence Listing which has been submitted electronically in ASCII format and is hereby incorporated by reference in its entirety. Said ASCII copy, created on Aug. 31, 2015, is named 47977_{—}00_{—}2001_SL.txt and is 138,917 bytes in size.

### FIELD OF THE INVENTION

The invention relates to methods for modifying the growth and other traits in plants by altering the SUMOylation status of a plant target protein.

### BACKGROUND OF THE INVENTION

The ever-increasing world population and the dwindling supply of arable land available for agriculture fuels research towards increasing the efficiency of agriculture. Conventional means for crop and horticultural improvements utilise selective breeding techniques to identify plants having desirable characteristics. However, such selective breeding techniques have several drawbacks, namely that these techniques are typically labour intensive and result in plants that often contain heterogeneous genetic components that may not always result in the desirable trait being passed on from parent plants. Advances in molecular biology have allowed mankind to modify the germplasm of animals and plants. Genetic engineering of plants entails the isolation and manipulation of genetic material (typically in the form of DNA or RNA) and the subsequent introduction of that genetic material into a plant. Such technology has the capacity to deliver crops or plants having various improved economic, agronomic or horticultural traits. A trait of particular economic interest is growth, in that it is a determinant of eventual crop yield.

Plants adapt to changing environmental conditions by modifying their growth. Plant growth and development is a complex process involves the integration of many environmental and endogenous signals that, together with the intrinsic genetic program, determine plant form. Factors that are involved in this process include several growth regulators collectively called the plant hormones or phytohormones. This group includes auxin, cytokinin, the gibberellins (GAs), abscisic acid (ABA), ethylene, the brassinosteroids (BRs), and jasmonic acid (JA), each of which acts at low concentrations to regulate many aspects of plant growth and development. Abiotic and biotic stress can negatively impact on plant growth leading to significant losses in agriculture. Even moderate stress can have significant impact on plant growth and thus yield of agriculturally important crop plants. Therefore, finding a way to improve growth, in particular under stress conditions, is of great economic interest. The inventors have found that altering the SUMOylation status of a protein results in desirable phenotypes which are of great benefit in agriculture.

Gibberellins (GA) play a key role regulating these adaptive responses by stimulating the degradation of growth repressing DELLA proteins (1-4). The current model for GA signaling describes how this hormone binds to its receptor GID1 so promoting association of GID1 with DELLA (5-10), which then undergoes ubiquitin-mediated proteasomal degradation (11-17). Current evidence indicates that a key strategy employed by plants to survive adverse conditions is to restrain growth via DELLA accumulation (1, 18). DELLA proteins are the central repressors of molecular pathways governed by the growth promoting phytohormone GA (19-22). Recently it was shown that DELLA protein levels are critical for the coordination of plant development by light and GA (23, 24). The integrative role of DELLAs is heavily reliant on the plant's ability to control cellular DELLA protein levels. Prior to this study the only mechanism for regulating DELLA protein abundance was through modulating the levels of GA to trigger ubiquitin-mediated proteasomal degradation.

Auxin Response Factors (ARFs) are transcriptional activators of early auxin response genes. ARFs bind to the auxin response elements (AuxREs) in the promoter region of early auxin response genes and activate or repress their transcription. ARF7 and ARF19 are key components in a developmental pathway regulating lateral root formation. arf7 arf19 double mutants exhibit a severely reduced lateral root formation phenotype not observed in arf7 and arf19 single mutants, indicating that lateral root formation is redundantly regulated by these two ARF transcriptional activators. The root system of higher plants consists of an embryonic primary root and postembryonic developed lateral roots and adventitious roots. In dicot plants, lateral root formation is crucial for maximizing a root system's ability to absorb water and nutrients as well as to anchor plants in the soil (44). Therefore, manipulating lateral root formation is a desirable goal in creating plants that are more able to withstand abiotic stress, for example drought or poor soil conditions.

Eukaryotic protein function is regulated in part by posttranslational processes such as the covalent attachment of small polypeptides. The most frequent and best characterized is the modification by ubiquitin and ubiquitin-like proteins. SUMO, the small ubiquitin-like modifier is similar to ubiquitin in tertiary structure but differs in primary sequence. SUMO conjugation to target proteins, a process referred to as SUMOylation, involves the sequential action of a number of enzymes, namely, activating (E1), conjugating (E2 or SUMO E2) and ligase (E3). The process is reversible, and desumoylation, that is, removal of SUMO from the substrate, is mediated by SUMO proteases. Mechanistically, SUMOylation comprises distinct phases. Initially the E1 enzyme complex activates SUMO by binding to it via a highly reactive sulfhydryl bond. Activated SUMO is then transferred to the E2 conjugating enzyme via trans-sterification reaction, involving a conserved cysteine residue in the E2 enzyme. Residue cysteine 94 is the conjugated residue in the Arabidopsis thaliana E2 enzyme, also named AtSCEI protein. In the last step, SUMO is transferred to the substrate via an isopeptide bond.

While protein modification by ubiquitin often results in protein degradation, SUMOylation, i.e. conjugation of SUMO to proteins, is often associated with protein stabilization. SUMOylation function is best understood in yeast and animals where it plays a role in signal transduction, cell cycle DNA repair, transcriptional regulation, nuclear import and subsequent localization and in viral pathogenesis. In plants, SUMOylation has been implicated in regulation of gene expression in response to development, hormonal and environmental changes (25).

Citation or identification of any document in this application is not an admission that such document is available as prior art to the present invention.

### SUMMARY OF THE INVENTION

In summary, being able to control growth responses in plants, for example hypocotyl/stem elongation, but also root growth, in particular to environmental cues, is of major importance in controlling yield, specifically in view of climate change which often leads to adverse environmental conditions. Applicants have identified methods and compositions which are aimed at meeting this need for providing plants with improved responses under stress and non-stress conditions and which are of agricultural benefit.

In a first aspect, the invention relates to a method for modifying growth, yield or root development of a plant which may comprise altering the SUMOylation status of a target protein or altering the interaction of a SUMOylated target protein with its receptor.

In one embodiment, the invention relates to a method for modifying growth of a plant under stress conditions which may comprise expressing a nucleic acid construct which may comprise a nucleic acid that encodes a mutant RGL1-, RGL-2, GAI, RGL-3 polypeptide, wherein the mutant polypeptide is as defined in SEQ ID No. 2, 6, 8 or 12 or a functional variant homologue or orthologue thereof but which may comprise a substitution of a conserved residue, for example the K residue, in the conserved SUMOylation site in a plant. The SUMOylation site is shown in FIG. 2d.

In a further aspect, the invention relates to a transgenic plant expressing a gene encoding for a mutant receptor protein which may comprise an altered SIM site wherein said unmodified receptor protein binds a target protein involved in growth regulation. In a further aspect, the invention relates to an isolated plant cell expressing a gene encoding for a mutant target protein involved in growth regulation wherein said protein may comprise an altered SUMOylation site. In a further aspect, the invention relates to an isolated plant cell expressing a gene encoding for a mutant receptor protein which may comprise an altered SIM site wherein said unmodified receptor protein binds a target protein involved in growth regulation. In yet a further aspect, the invention relates to a method for increasing growth which may comprise altering the SUMOylation status of a target protein or altering the interaction of a SUMOylated target protein with its receptor. The invention also relates to a method for increasing stress tolerance which may comprise altering the SUMOylation status of a target protein or altering the interaction of a SUMOylated target protein with its receptor. In a further aspect, the invention relates to an in vitro assay for identifying a target compound that increases SUMOylation. The invention also relates to a method for identifying a compound that regulates SUMOylation and methods for using such compound sin altering SUMOylation of a target protein.

In another aspect, the invention relates to a method for altering root architecture, by manipulating SUMOylation of a AtARF19 or AtARF7 polypeptide as defined in SEQ ID No. 14 or 16, a functional variant, homolog or ortholog thereof and introducing and expressing an altered ARF19 or ARF7 nucleic acid encoding for a mutant protein in a plant. In a further aspect, the invention relates to a transgenic plant obtained or obtainable by one of the methods described herein. The invention also relates to a transgenic plant expressing a gene encoding for a mutant target protein selected from a RGL-1, RGL-2, GAI, RGL-3 polypeptide, a homologue or orthologue thereof involved in growth regulation and/or expressing a gene encoding for a mutant target protein selected from a ARF7 or ARF19 polypeptide involved in the development of root architecture wherein said protein may comprise an altered SUMOylation site or additional SUMOylation sites.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. §112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. Nothing herein is to be construed as a promise.

It is noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following detailed description, given by way of example, but not intended to limit the invention solely to the specific embodiments described, may best be understood in conjunction with the accompanying drawings.

FIGS. 1A-D. OTS1 and OTS2 modulate growth through a DELLA-dependent mechanism. a, Images of NaCl-grown seedlings. Bar=5 mm. b, mean root growth on 100 mM NaCl expressed as an inhibition (%) relatively to the untreated controls. Error bar=s.e.m. n=20-24. c, accumulation of RGA protein in the absence (−) or presence (+) of 100 mM NaCl. Number indicates molecular mass (kDa). Coomassie Blue filter staining (C. Blue) serves as a loading control. d, mean concentrations of gibberellins (GAs) in ots1 ots2 double mutants and wild type (Col-0). Error bars=s.d. of 3 biological replicates.

FIGS. 2A-G. SUMOylation is a novel DELLA modification affecting DELLA accumulation. a, Immunoprecipitation of GFP:RGA proteins. Arrow indicates the GFP:RGA protein, vertical bars, the SUMOylated forms of GFP:RGA protein. b, in vitro deSUMOylation of plant-derived GFP:RGA with recombinant His:OTS1 or His:OTS1C526S. c, His:RGA SUMOylation in E. coli by activating (E1), conjugating (E2) enzymes and active (His:AtS1GG) but not inactive (His:AtS1AA) AtSUMO1. His:RGAK65R is not SUMOylated. Arrow reveals the SUMOylated forms of His:RGA protein. d, crossspecies alignment of the DELLA domain (“DELLA” disclosed as SEQ ID NO: 70). In bold characters, the conserved lysine residues, shaded area, the non-canonical SUMOylation motif. Alignment discloses SEQ ID NOS 41, 76-77, 42, and 78-82, respectively, in order of appearance. e, SUMOylated GFP:RGA accumulation upon NaCl treatment. f, SUMOylated GFP:RGA accumulation in wild-type (OTS1 OTS2) or mutants (ots1 ots2) plants. g, accumulation of GFP:RGA at different concentrations of NaCl. Wild-type extracts (Col-0) were used as a negative control.

FIGS. 3A-F. DELLA deSUMOylation impairs DELLA accumulation. a, Images of 20 days-old petri-grown seedlings. b, accumulation of RGA or GAI proteins in wild-type (Ler), gal-5 or three transgenic (T2) 35S::4Xmyc:OTS2 gal-5 lines. RGA* indicates a cross reaction of the GAI antibody with RGA. c, real-time PCR analysis of RGA, GAI and OTS2 transcripts levels. Total RNA derived from the same samples as in b. Bars indicate the expression levels as fold change variations relatively to gal-5. ACTIN was used for normalisation, error bars=s.d. of two technical replicates. d, Image of 8 weeks T1 transgenic plants (gal-5 background). e, accumulation of RGA, RGA:GFP or RGAK65R:GFP proteins from transgenic (T2) seedlings. Longer exposure (bottom) reveals the endogenous RGA protein. f, real-time PCR analysis of RGA transcripts levels. RNA derived from the same samples as in e. Bars indicate the expression levels relatively to vector control line #1. Error bars=s.d. of two technical replicates.

FIGS. 4A-F. SUMOylated DELLA binds GID1 independently from GA. a, crossspecies alignment of SIM B in the GID1 protein amino terminal extension (grey). Alignment discloses SEQ ID NOS 50-55, respectively, in order of appearance. b, GST pull down assay between His:AtSUMO1 and GST:GID1a or GST in the presence (+) or absence (−) of GA3 (10 μM). Asterisk indicates a cross-reacting band. c, GST pull down assay between plant-derived GFP:RGA proteins with recombinant GST:GID1a or GST. d, mean germination rates (percentage of visible green cotyledons) of wild type (wt), ots1 ots2 double mutants and transgenic lines (T4). n=40-80 for each treatment/genotype combination. Error bar=s.d. of three biological replicates. e, images of NaCl-grown seedlings. Bar=1 cm f, model for the SUMOylation-dependent DELLA accumulation.

FIGS. 5A-C. OTS1 and OTS2 mediate GA signaling through DELLA. a, Image of germinating seeds photographed 5 days after sowing in the presence or absence of PAC. b and c, mean germination rates (percentage of visible green cotyledons) under different PAC or PAC and/or gibberellic acid (GA3) concentrations. n=11 40-80 for each treatment/genotype combination. Error bar=s.d. of three biological replicates.

FIGS. 6A-B. Increased DELLA protein levels in ots1 ots2 is not dependent on altered DELLA transcripts levels. a, immunoblot detection of GAI protein in 10 days old seedlings of the indicated genotypes grown in petri dishes in the presence of different concentrations of NaCl. Coomassie Blue filter staining (C. Blue) serves as a loading control. b, real-time PCR analysis of RGA and GAI transcripts levels in the presence or absence of 100 mM NaCl. Bars indicate the expression levels as fold change variations relatively to wild-type control samples (which was arbitrarily set as 1). ACTIN was used for normalisation, error bars=s.d. of two biological replicate, each one performed in two technical replicates. ND=not detected.

FIGS. 7A-D. RGA and GAI are SUMOylated in vivo. a, Immunoprecipitation of GFP proteins from 35S::GFP or 35S::GFP:NPR1 (NON EXPRESSER OF PR GENES) young seedlings sprayed with 1 mM Salicylic acid (+SA) or control (−SA). Numbers indicate molecular mass (kDa), arrowhead, the GFP:NPR1 or GFP proteins. Ponceau staining of the Rubisco large subunit serves as a loading control. b, in vitro deSUMOylation of plant-derived GFP:RGA by recombinant SUMO protease subunits of SENP1 and SENP2. c, immunoprecipitation of equal amount of total proteins derived from pRGA::GFP:RGA seedlings or a transgenic line (Col-0) expressing GAI:GFP (35S::GAI:GFP). 9 days old seedlings were grown in petri dishes in the absence (−) or presence (+) of PAC (0.1 μM). Immunoprecipitated proteins were probed with GFP (WB aGFP) or AtSUMO1 (WB aAtS1) antibodies. The migration of GFP:RGA, GAI:GFP and their respective SUMOylated forms is shown. d, immunoprecipitation of GFP:RGA proteins derived from pRGA::GFP:RGA seedlings, harvested at different time point (hours) after being sprayed with GA3 (10 μM) and compared to untreated control (ctrl). The migration of GFP:RGA and SUMOylated forms (AtS1-GFP:RGA) of GFP:RGA protein is indicated.

FIGS. 8A-D. SUMOylation affects DELLA activity in vivo. a, mean rosette size (maximum diameter) of 24 days old wild-type (Ler), gal-5 and transgenic (T2) plants grown on soil. n=16-18, Bar=s.e.m. b, images of 6 weeks old wild-type (Ler), gal-5 and 35S::4Xmyc:OTS2 gal-5 #3 transgenic (T2) plants. Inset shows gal-5 and 35S::4Xmyc:OTS2 plants one week later. Note the increased stem length and presence of open flowers and developing siliques in the transgenic line but not in the gal-5 mutant. Scale bar=1 cm. c, plant height phenotypic classes of T1 transgenic plants (gal-5 background) transformed with empty vector (Vector), 35S::RGA:GFP, or 35S::RGAK65R:GFP. The primary inflorescences of independent Basta resistant plants were measured after 8 weeks of growth on soil. d, flowering time phenotypic classes of T1 plants as illustrated in c.

FIGS. 9A-C. GID1a contains a functional SIM motif in the N-terminal region. a, amino acid positions of two putative SIMs (SUMO interacting motifs) in the GID1a N-terminal domain (SEQ ID NO: 59). Lower panel, far-western assays of two peptides corresponding to SIM A (SEQ ID NO: 60) and SIM B (SEQ ID NO: 58). Binding between the SIM and SUMO1 occurs with SIM B. SIMs contain a central, mostly hydrophobic, core (bold character). The substitution of a hydrophobic amino acid for an alanine residue (SIM B V22A) results in a strongly reduced SIM-SUMO1 interaction. b, immunoblot detection of GID1a:TAP protein derived from independent transgenic 35S::GID1a:TAP young seedlings. Number indicates molecular mass (kDa). Non-transgenic, wild-type extracts (wt) were used as a negative control. c, mean root growth of 10 days old seedlings in the presence of 100 mM NaCl expressed as a inhibition (%) relatively to the untreated controls. Error bar=s.e.m. n=16.

FIGS. 10A-B. SIMs are conserved in crop species Peptide arrays to identify SIMs in GID1 proteins. a) Initial screening of two putative SIMs (SEQ ID NOS 57 and 61) in AtGID1a (SEQ ID NO: 56), showing location and sequence; SIM “B” shown to be a genuine SIM and the V22A mutant of this SIM shows a reduction in interaction. b) Peptide array of all SIMs in Arabidopsis, rice and maize; all show interaction with SUMO1; all W21A mutations show reduced interaction while the V22T mutations had little effect except for AtGID1b.

FIG. 11. Sequence alignment of DELLA proteins. DELLA proteins from different species are highly conserved. The figure shows sequences for DELLA proteins for Arabidopsis (AtRGA (SEQ ID NO: 62), AtGAI (SEQ ID NO: 63)), rice (OsSLN) (SEQ ID NO: 64), maize (ZmD8) (SEQ ID NO: 83) and wheat (TaRht) (SEQ ID NO: 65). Also shown is the consensus sequence.

FIG. 12. JAZ proteins are SUMOylated. Western blot of SUMOylation screen of JAZ6, with three K to R mutants. Arrows indicate SUMOylation band shifts. Blot shows that JAZ6 is SUMOylated and that mutating lysine 221 to arginine (K221R) abolishes SUMOylation, therefore lysine 221 is likely the site of SUMOylation. JAZ6 fused to maltose binding protein (MBP) and probed with anti MBP.

FIG. 13. PHY-B (S86D) phospho mutant is not SUMOylated. A SUMOylation screen of phytochrome B (PHYB-GFP), with two mutant forms, PHY-B (S86D), which is the hyperphosphorylated form of PHYB, and PHY-B S86A, the non phosphorylated form was carried out by Western Blot. Arrows indicate SUMOylation band shifts. Blot shows that PHY-B is hyperSUMOylated during middle of day then end of night. The hyperphosphorylated mutant form cannot be SUMOylated even in the middle of day time point indicating interdependence of phosphorylation and SUMOylation mechanisms.

FIG. 14. Transgenic plants expressing mutated forms of DELLA proteins
1: 35S::RGA (k/r):GFP
2: 35S::RGA:GFP
3: 35S::GAI:GFP
4: 35S::GAI(k/r):GFP
5: 35S:GFP
6: Col-0-

FIGS. 15A-C. Expression of a GID SIM mutant
a) Expression of 35S:GID1a and 35S:GID1a (V22A) in the ots1:ots2 background in the absence of salt.
b) Expression of 35S:GID1a and 35S:GID1a (V22A) in the ots1:ots2 background in the presence of salt (75 mM NaCl).
c) Expression of 35S:GID1a and 35S:GID1a (V22A) in wt background in the presence of salt (75 mM NaCl).

FIGS. 16A-C. ARF19 and ARF7 are sumoylated a) GST-ARF7/19 SUMOylation in E. coli by activating (E1), conjugating (E2) enzymes; b) ARF19 protein levels are up regulated in ots1/2 SUMO protease mutants ; c) ARF 7/19 SUMO sites are missing in rice (SEQ ID NOS 66-69, respectively, in order of appearance).

FIG. 17. SUMO inhibits GID1a binding to RGA-DELLA protein Interaction between RGA alone with GID1a (red) and, RGA and SUMO1 (AtS1, blue) with GID1a both in the presence of GA3. The combined response (blue) is reduced in the presence of AtS1 indicating that less of the higher molecular weight RGA is bound, being displaced by the lower molecular weight AtS1. Shaded area shows SE (standard error of the mean). Method: SPR was carried out on a Biacore 2000 instrument at 25° C. Purified GID1a was amine-coupled to a CM5 sensor chip (GE Healthcare). Flow cell 1 was blocked using ethanolamine and used as reference. Approx 500 RU of GID1a was bound to flow cells 2 and 3. All binding assays were carried out in HBS-EP buffer (10 mM HEPES pH 7.5, 150 mM NaCl, 1 mM EDTA, 1 mM DTT, 0.005% P20) at a flow rate of 20 μl/min using 180 second injections followed by 180 s of dissociation in HBS-EP. Each condition was run in duplicate using proteins at 100 μg/ml in HBS-EP (containing 100 μM GA3 as appropriate). Regeneration used 10 mM glycine pH 1.5 at 30 μl/min for 30 s.

FIGS. 18A and B. GID1a—SUMO Interaction Data Sensorgram of interaction between SUMO1 (AtS1) with GID1a. Figure shows binding and saturation of AtS1 to GID1a followed by disassociation when AtS1 is removed from buffer flow over GID1a. Shaded area shows SE (standard error of the mean).

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The practice of the present invention will employ, unless otherwise indicated, conventional techniques of botany, microbiology, tissue culture, molecular biology, chemistry, biochemistry and recombinant DNA technology, bioinformatics which are within the skill of the art. Such techniques are explained fully in the literature.

The inventors have shown that altering the SUMOylation status of a target protein in a plant modifies growth. Thus, the invention relates to methods for altering growth of a plant which may comprise altering the SUMOylation status of a target protein. The invention further provides transgenic plants with altered growth which express a nucleic acid that encodes a mutant target protein that has a decrease or increase in its susceptibility to SUMOylation. In other words, the mutant target protein is SUMOylated to a greater or lesser extent. The invention also provides transgenic plants with altered growth which express a nucleic acid that encodes a mutant receptor protein which has reduced or increased susceptibility for interaction with its SUMOylated target protein. The invention also relates to isolated nucleic acid sequences and uses thereof.

As used herein, the words “nucleic acid”, “nucleic acid sequence”, “nucleotide”, “nucleic acid molecule” or “polynucleotide” are intended to include DNA molecules (e.g., cDNA or genomic DNA), RNA molecules (e.g., mRNA), natural occurring, mutated, synthetic DNA or RNA molecules, and analogs of the DNA or RNA generated using nucleotide analogs. It can be single-stranded or double-stranded. Such nucleic acids or polynucleotides include, but are not limited to, coding sequences of structural genes, anti-sense sequences, and non-coding regulatory sequences that do not encode mRNAs or protein products. These terms also encompass a gene. The term “gene” or “gene sequence” is used broadly to refer to a DNA nucleic acid associated with a biological function. Thus, genes may include introns and exons as in genomic sequence, or may comprise only a coding sequence as in cDNAs, and/or may include cDNAs in combination with regulatory sequences. In some embodiment, the DNA of the nucleic acids described herein explicitly refers to cDNA. Thus, in the various methods described herein, the nucleic acid is, in one embodiment, cDNA of genomic sequence listed herein.

The terms “polypeptide” and “protein” are used interchangeably herein and refer to amino acids in a polymeric form of any length, linked together by peptide bonds.

For the purposes of the invention, “transgenic”, “transgene” or “recombinant” means with regard to, for example, a nucleic acid sequence, an expression cassette, gene construct or a vector which may comprise the nucleic acid sequence or an organism transformed with the nucleic acid sequences, expression cassettes or vectors according to the invention, all those constructions brought about by recombinant methods in which either
(a) the nucleic acid sequences encoding proteins useful in the methods of the invention, or
(b) genetic control sequence(s) which is operably linked with the nucleic acid sequence according to the invention, for example a promoter, or
(c) a) and b)

are not located in their natural genetic environment or have been modified by recombinant methods, it being possible for the modification to take the form of, for example, a substitution, addition, deletion, inversion or insertion of one or more nucleotide residues. The natural genetic environment is understood as meaning the natural genomic or chromosomal locus in the original plant or the presence in a genomic library. In the case of a genomic library, the natural genetic environment of the nucleic acid sequence is preferably retained, at least in part. The environment flanks the nucleic acid sequence at least on one side and has a sequence length of at least 50 bp, preferably at least 500 bp, especially preferably at least 1000 bp, most preferably at least 5000 bp. A naturally occurring expression cassette—for example the naturally occurring combination of the natural promoter of the nucleic acid sequences with the corresponding nucleic acid sequence encoding a polypeptide useful in the methods of the present invention, as defined above—becomes a transgenic expression cassette when this expression cassette is modified by non-natural, synthetic (“artificial”) methods such as, for example, mutagenic treatment. Suitable methods are described, for example, in U.S. Pat. No. 5,565,350 or WO 00/15815.

A transgenic plant for the purposes of the invention is thus understood as meaning, as above, that the nucleic acids used in the method of the invention are not at their natural locus in the genome of said plant, it being possible for the nucleic acids to be expressed homologously or heterologously. However, as mentioned, transgenic also means that, while the nucleic acids according to the different embodiments of the invention are at their natural position in the genome of a plant, the sequence has been modified with regard to the natural sequence, and/or that the regulatory sequences of the natural sequences have been modified. Transgenic is preferably understood as meaning the expression of the nucleic acids according to the invention at an unnatural locus in the genome, i.e. homologous or, preferably, heterologous expression of the nucleic acids takes place. Preferably, according to the methods described herein, the progeny plant is stably transformed and may comprise the exogenous polynucleotide which is heritable as a fragment of DNA maintained in the plant cell and the method may include steps to verify that the construct is stably integrated. The method may also comprise the additional step of collecting seeds from the selected progeny plant and producing a food or feed composition.

The plant according to the various aspects of the invention may be a moncot or a dicot plant. A dicot plant may be selected from the families including, but not limited to Asteraceae, Brassicaceae (eg Brassica napus), Chenopodiaceae, Cucurbitaceae, Leguminosae (Caesalpiniaceae, Aesalpiniaceae Mimosaceae, Papilionaceae or Fabaceae), Malvaceae, Rosaceae or Solanaceae. For example, the plant may be selected from lettuce, sunflower, Arabidopsis, broccoli, spinach, water melon, squash, cabbage, tomato, potato, yam, capsicum, tobacco, cotton, okra, apple, rose, strawberry, alfalfa, bean, soybean, field (fava) bean, pea, lentil, peanut, chickpea, apricots, pears, peach, grape vine or citrus species. In one embodiment, the plant is oilseed rape.

Also included are biofuel and bioenergy crops such as rape/canola, sugar cane, sweet sorghum, Panicum virgatum (switchgrass), linseed, lupin and willow, poplar, poplar hybrids, Miscanthus or gymnosperms, such as loblolly pine. Also included are crops for silage (maize), grazing or fodder (grasses, clover, sanfoin, alfalfa), fibres (e.g. cotton, flax), building materials (e.g. pine, oak), pulping (e.g. poplar), feeder stocks for the chemical industry (e.g. high erucic acid oil seed rape, linseed) and for amenity purposes (e.g. turf grasses for golf courses), ornamentals for public and private gardens (e.g. snapdragon, petunia, roses, geranium, Nicotiana sp.) and plants and cut flowers for the home (African violets, Begonias, chrysanthemums, geraniums, Coleus spider plants, Dracaena, rubber plant).

A monocot plant may, for example, be selected from the families Arecaceae, Amaryllidaceae or Poaceae. For example, the plant may be a cereal crop, such as wheat, rice, barley, maize, oat, sorghum, rye, millet, buckwheat, turf grass, Italian rye grass, sugarcane or Festuca species, or a crop such as onion, leek, yam or banana.

Preferably, the plant is a crop plant. By crop plant is meant any plant which is grown on a commercial scale for human or animal consumption or use. Preferred plants are maize, wheat, rice, oilseed rape, sorghum, soybean, potato, tomato, grape, barley, pea, bean, field bean, lettuce, cotton, sugar cane, sugar beet, broccoli or other vegetable brassicas or poplar.

The term “plant” as used herein encompasses whole plants, ancestors and progeny of the plants and plant parts, including seeds, fruit, shoots, stems, leaves, roots (including tubers), flowers, and tissues and organs, wherein each of the aforementioned may comprise the gene/nucleic acid of interest. The term “plant” also encompasses plant cells, suspension cultures, callus tissue, embryos, meristematic regions, gametophytes, sporophytes, pollen and microspores, again wherein each of the aforementioned may comprise the gene/nucleic acid of interest. The invention furthermore relates to products derived, preferably directly derived, from a harvestable part of such a plant, such as dry pellets or powders, oil, fat and fatty acids, starch or proteins, including food and animal feed compositions.

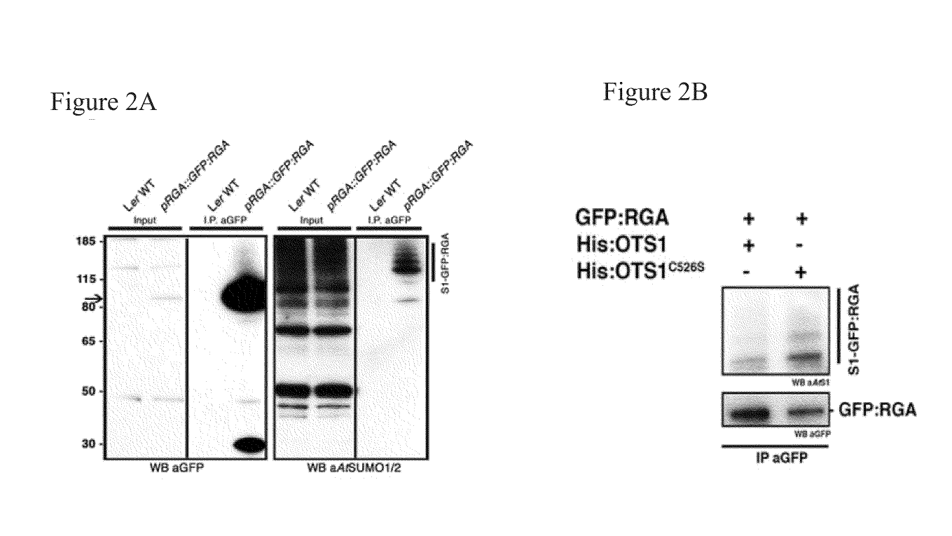

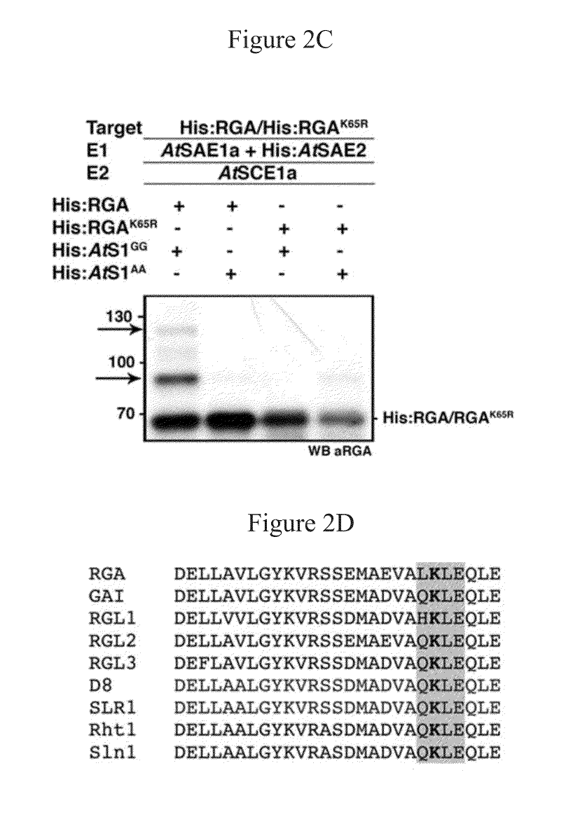

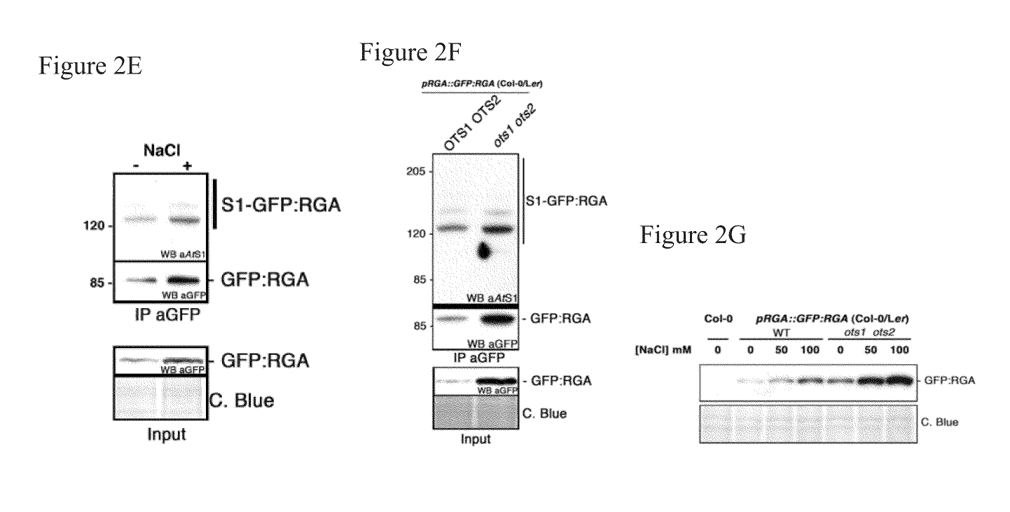

The examples demonstrate in vivo transformation of Arabidopsis thaliana. However, a skilled person would know that the invention can be applied to other plant species by routine experimentation. Arabidopsis thaliana is a well known model plant that has been used in numerous biotechnological processes and it has been demonstrated that the results obtained in Arabidopsis thaliana can be extrapolated to any other plant species. This is in particular the case for signaling processes that are conserved in the plant kingdom, as for example in the case of signaling involving DELLA proteins. DELLA proteins are those that are characterised by a DELLA amino acid motif (“DELLA” disclosed as SEQ ID NO: 70) as shown in FIG. 2.

Furthermore, according to some embodiments of the various aspects of the invention that concern the expression of a transgene in a plant, the gene that is expressed in the plant encodes for an endogenous protein. For example, a wheat DELLA protein (TaRht1) may be expressed in a wheat plant as part of an expression cassette using recombinant technology. In another embodiment, the gene encodes for an exogenous protein. For example, an Arabidopsis GAI protein may be expressed in a different plant species, for example a crop plant, as part of an expression cassette using recombinant technology.

In a first aspect, the invention relates to a method for modifying growth of a plant which may comprise altering the SUMOylation status of a target protein. In one embodiment, this increases yield.

The term SUMOylation status refers to the degree of SUMOylation of a target protein or its susceptibility to SUMOylation. In one embodiment, the SUMOylation status refers to the degree of SUMOylation of a target protein, that is the presence or absence of SUMOylation sites.

In one preferred embodiment of all of the various aspects of the invention, growth is modified under abiotic stress conditions. Abiotic stress is preferably selected from drought, salinity, freezing, low temperature or chilling. In one embodiment, the stress is moderate or mild stress, for example moderate salinity. Thus, the invention relates to improving growth of a plant under moderate or severe abiotic stress conditions which may comprise altering the SUMOylation status of a target protein. Under moderate stress conditions, this yields plants that show improved growth under stress conditions under which growth of control plants normally is impaired. Thus, the invention also relates to mitigating the effects of abiotic stress on plant growth by altering the SUMOylation status of a target protein as described herein.

In one embodiment, a target protein is a protein that is involved in growth regulation and which may comprise a SUMOYlation site. For example, the protein may be a component of a plant hormone signaling pathway. This pathway includes auxin, cytokinin, GA, ABA, ethylene, BR and JA signaling. Other genes known to influence growth include, but are not limited to, JAZ proteins, including JAZ6, ABI3, ABI5, DELLAs proteins, PHYB, PHYA, PHYC, PHYD PHOT1, PHOT2, PIF proteins, SPT1, CTS, PIL5, PYL5, PYL7, NPR1, BHLH32, FT, CO, BAK1, CERK1, FLS2, EIN1, EIN2, ARF7 and ARF19. In one embodiment of the various aspects of the invention, the proteins that are included in the ABA pathway, such as ABI, for example ABI5, are specifically disclaimed.

In one embodiment of the various aspects of the invention, growth may be increased compared to a control plant. In another embodiment, growth may be repressed compared to a control plant. A control plant is a plant in which the SUMOylation status of a target protein has not been altered and/or in which binding of a SUMOylated target protein to its receptor has not been altered, for example a wild type plant. The control plant is preferably of the same species. Furthermore, the control plant may comprise additional genetic modifications that do however not affect SUMOylation.

In a preferred aspect of the method for altering growth, growth is increased compared to a control plant. Thus, the invention also relates to a method for increasing growth of a plant which may comprise altering the SUMOylation status of a target protein. According to this aspect of the invention, an increase in growth can be achieved in different ways. In one preferred embodiment, SUMOylation of a target protein is decreased or prevented. In another embodiment, SUMOylation of a target protein is increased.

The terms “increase”, “improve” or “enhance” are interchangeable. Growth or yield is increased by at least a 3%, 4%, 5%, 6%, 7%, 8%, 9% or 10%, preferably at least 15% or 20%, more preferably 25%, 30%, 35%, 40% or 50% or more in comparison to a control plant. Preferably, growth is measured by measuring hypocotyl or stem length. The term “yield” in general means a measurable produce of economic value, typically related to a specified crop, to an area, and to a period of time. Individual plant parts directly contribute to yield based on their number, size and/or weight, or the actual yield is the yield per square meter for a crop and year, which is determined by dividing total production (includes both harvested and appraised production) by planted square meters. The term “yield” of a plant may relate to vegetative biomass (root and/or shoot biomass), to reproductive organs, and/or to propagules (such as seeds) of that plant. Thus, according to the invention, yield may comprise one or more of and can be measured by assessing one or more of: increased seed yield per plant, increased seed filling rate, increased number of filled seeds, increased harvest index, increased number of seed capsules/pods, increased seed size, increased growth or increased branching, for example inflorescences with more branches. Preferably, yield may comprise an increased number of seed capsules/pods and/or increased branching. Yield is increased relative to control plants.

SUMOylation is increased by adding 1, 2, 3, 4, 5 or more additional SUMOylation sites to a target protein as described below.

In one embodiment, the method may comprise decreasing or preventing SUMOylation of a target protein. For example, SUMOylation of the target protein is prevented by expressing a nucleic acid sequence encoding a mutant target protein in a plant wherein said nucleic acid sequence has been altered to prevent or reduce SUMOylation of said target protein.

It is known that SUMOylation requires interaction between the substrate (target protein) and SUMO. Three enzymes mediate covalent attachment of SUMO to substrate proteins: SUMO-activating enzyme (SAE or E1), SUMO-conjugating enzyme (SCE or E2), and SUMO ligase (E3). SAE, a heterodimer (SAE1 and SAE2), forms a thioester bond between a reactive cysteine residue in its large subunit (SAE2) and the C-terminal end of SUMO. SCE binds both SUMO and the potential substrate and mediates the transfer and conjugation of SUMO from SAE to the substrate. Specific residues in SCE interact with a sequence motif present in the substrate called the SUMO attachment site (SAS). The term “motif or “consensus sequence” or “signature” refers to a short conserved region in the sequence of evolutionarily related proteins. Motifs are frequently highly conserved parts of domains, but may also include only part of the domain, or be located outside of conserved domain (if all of the amino acids of the motif fall outside of a defined domain). As described in the art, one SAS consensus sequence or SUMOylation motif that has been identified in plants typically consists of a lysine residue to which SUMO is attached (position 2), flanked by preferably a hydrophobic amino acid (position 1), any amino acid (position 3), and an acidic amino acid (position 4), typically E or D (ΨKXE/D). SCE catalyzes the formation of an isopeptide bond between the ε-amino group of the lysine residue of the substrate and the C-terminal glycine residue of SUMO (25).

There are however also non-consensus SUMOylation motifs (i.e. not ΨKXE/D described above). These include:
(ICM) inverted consensus motif where the consensus site is inverted, but still maintains hydrophobic residues;
PDSM: a phosphorylation-dependent SUMO motif, where the phosphorylated serine is located at 5 amino acids distance from the modified lysine, a negatively charged amino acid-dependent SUMO motif (NDSM) and
a hydrophobic cluster SUMOylation motif (HCSM) that increases the efficiency of modification in relevant targets of SUMOylation.

Thus, to decrease or prevent SUMOylation according to the methods of the invention, one or more SUMOylation site within the target protein is altered to decrease the degree of SUMOylation. In one embodiment, SUMOylation is prevented and SUMO can no longer be conjugated to the target protein. This means that SUMOylation is substantially abolished. For example, site-directed mutagenesis of a target nucleic acid sequence encoding for a target protein can be used to substitute one or all SUMOylation sites to a non-SUMOylatable site or to delete one or more residues in the SUMOylation site. The amino acid substitutions are preferably conservative amino acid substitutions. Conservative substitution tables are well known in the art. Alternatively, insertions can be made to render the site non-functional.

In one embodiment, the conserved SUMOylation motif ΨKXE/D is changed. These changes preferably may comprise altering a codon encoding the conserved lysine (K) residue in this motif within the target nucleic acid by replacing a nucleotide within said codon to produce a protein with non-SUMOylatable residue. In other words, the codon encoding K is altered so that it encodes for a different amino acid, for example R. As shown in the examples, mutagenesis of the conserved SUMOylatable R in a target protein prevents SUMOylation of said protein.

Preferably, the conserved K residue is located within the following consensus SUMOylation motif: X₁/ΨKX₂E/D wherein the first residue in the motif is occupied by any amino acid (X₁) or a hydrophobic amino acid, X₂ is any amino acid and the final residue in the motif is E or D. The hydrophobic amino acid may be V, I, L, M, F, W, C, A, Y, H, T, S, P, G, R or K. In one embodiment, the first residue is not hydrophobic and X₁ is Q.

In one embodiment, further residues within the SUMOylation motif, in addition to K, may be altered by mutating one or more, for example all of the codons encoding for the remaining residues in the SUMOylation motif.

The mutant nucleic acid in which the codon encoding the SUMOylation acceptor K and/or another residue in the conserved SUMOylation site is altered can be expressed in a transgenic plant as part of an expression cassette which may comprise a promoter as described herein. This leads to abundance or targeted expression of non-SUMOylatable target protein which in turn increases growth of the transgenic plant compared to a control plant.

Amino acid substitutions, deletions and/or insertions may readily be made using peptide synthetic techniques well known in the art, such as solid phase peptide synthesis and the like, or by recombinant DNA manipulation. Methods for the manipulation of DNA sequences to produce substitution, insertion or deletion variants of a protein are well known in the art. For example, techniques for making substitution mutations at predetermined sites in DNA are well known to those skilled in the art and include M13 mutagenesis, T7-Gen in vitro mutagenesis (USB, Cleveland, Ohio), QuickChange Site Directed mutagenesis (Stratagene, San Diego, Calif.), PCR-mediated site-directed mutagenesis or other site-directed mutagenesis protocols.

Bioinformatics analysis can be used to predict SUMOylation sites in plant proteins based on the consensus motif X₁/ΨKX₂E/D. The key residue in the consensus motif is the K acceptor. Once a SUMOylation site in a plant protein from a specific species with a K acceptor has been predicted by bioinformatics and the use of protein sequence databases, further bioinformatics analysis can be carried out to confirm that the motif and in particular the K residue is conserved across homologues in a diverse range of plant species. As shown in the examples, this methodology was used to successfully predict the SUMO site in DELLA proteins. A skilled person would therefore be able to apply this method to identify SUMOylation sites in other growth regulating proteins.

It is known that although SUMOylation often occurs on specific K residues within the consensus SUMOylation motif other modifications, such as phosphorylation, may regulate the SUMOylation of a substrate. Therefore, according to the methods of the invention, the SUMOylation status of a target protein can be modified by reducing the degree of phosphorylation or preventing or increasing phosphorylation of the target protein.

In another embodiment, one or more of the non-consensus SUMOylation motifs listed above is altered.

In one embodiment of the methods for increasing growth by preventing SUMOylation according to the methods of the invention, phosphorylation-dependent SUMOylation of the target protein is decreased or prevented.

For example, phosphorylation-dependent SUMOylation of the target protein is prevented by expressing a nucleic acid sequence encoding a mutant target protein in a plant wherein said nucleic acid sequence has been altered to prevent phosphorylation-dependent SUMOylation of said target protein. This can be achieved by targeting one or more conserved residues which regulates phosphorylation-dependent SUMOylation. Mutating such a residue abolishes phosphorylation-dependent SUMOylation.

For example, PDSM (phosphorylation-dependent sumoylation motif), composed of a SUMO consensus site and an adjacent proline-directed phosphorylation site is a highly conserved bipartite motif that regulates phosphorylation-dependent sumoylation of multiple substrates, such as heat-shock factors (HSFs), GATA-1, and myocyte enhancer factor 2. PDSM may comprise a SUMOylation and a serine/proline directed phosphorylation site separated from the SUMOylation by one to seven amino acids. SUMOylation of the K residue in the SUMOylation motif is phosphorylation dependent. The target protein is first phosphorylated at the serine (S) residue and K is then SUMOylated. Accordingly, expressing a mutant nucleic acid in which the codon encoding the conserved S residue 1-7 amino acids downstream of the SUMOylation is mutated in a transgenic plant results in a protein which can no longer be SUMOylated.

In one embodiment of the methods of the invention, a mutant nucleic acid is expressed in a transgenic pant which may comprise a modified SUMOylation motif as described above and a modified phosphorylation site as described above.

It is known that there is a link to SUMOylation via glycosylation. For example in cases where phosphorylation affects SUMOylation, either by enhancing SUMOylation or preventing target SUMOylation, glycosylation is important as glycosylation has been shown to affect phosphorylation of target proteins (26). Thus, in one embodiment of the methods for increasing growth by preventing SUMOylation according to the methods of the invention, glycosylation-dependent SUMOylation of the target protein is decreased or prevented.

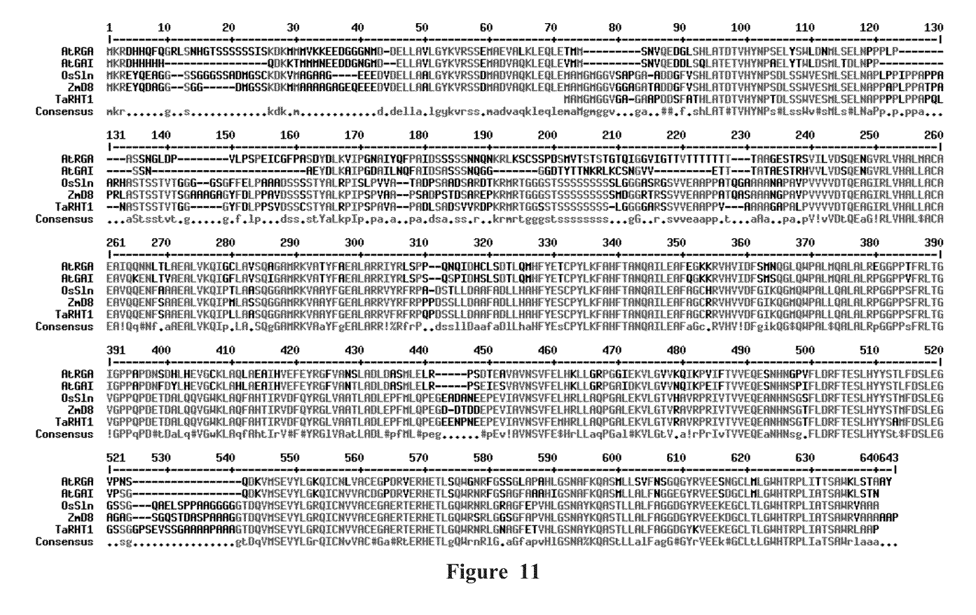

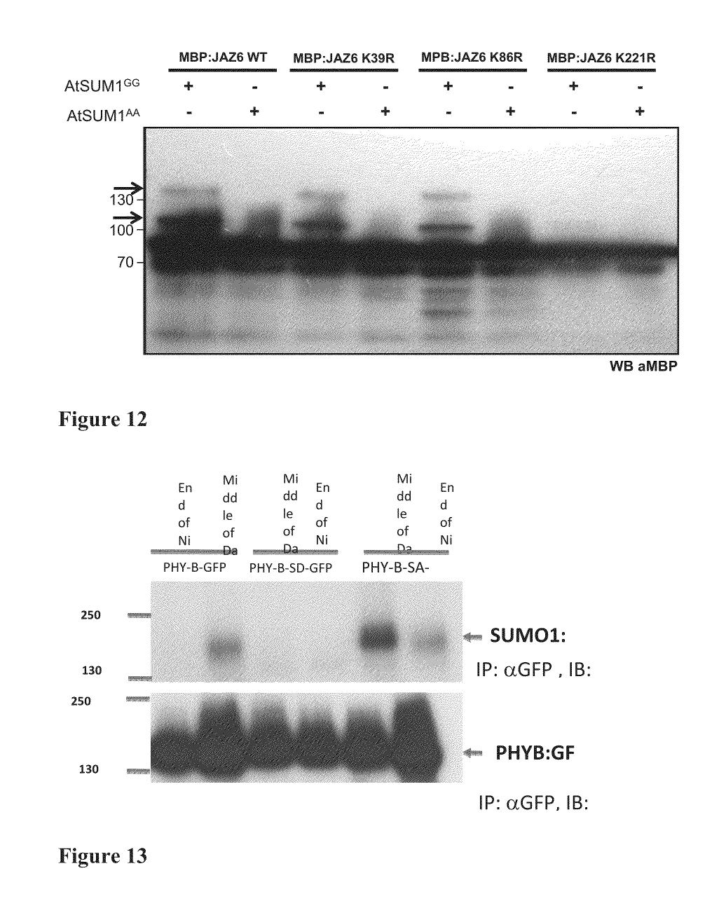

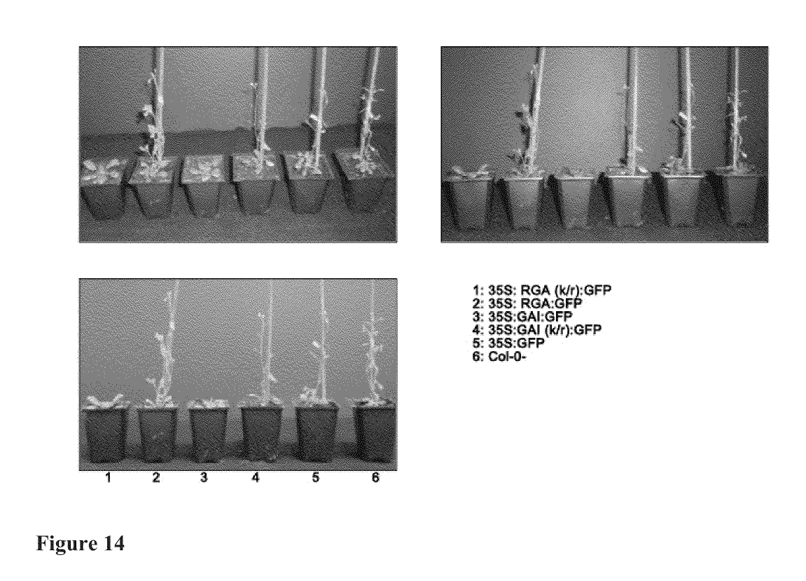

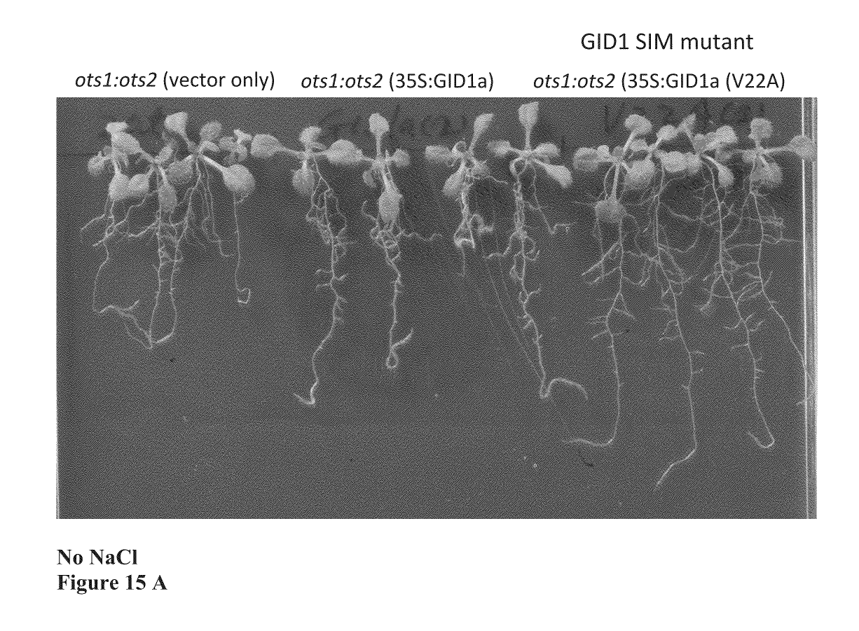

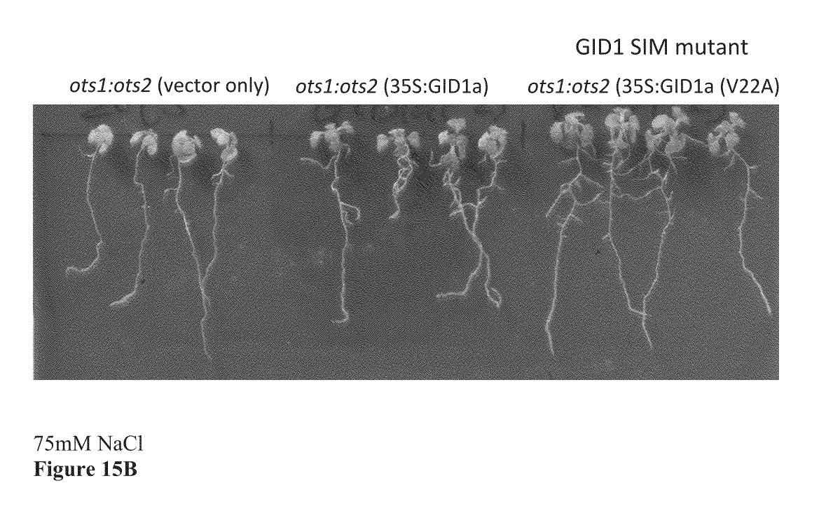

In one embodiment of the various aspects of the invention, the target protein is selected from a DELLA protein wherein said DELLA protein is not RGA. Thus, the DELLA protein is GAI or a GAI-like DELLA protein. A GAI-like protein refers for example to a protein that may comprise a DELLA domain (“DELLA” disclosed as SEQ ID NO: 70) and does, when overexpressed in a plant, result in a dwarf phenotype. DELLA proteins are involved in growth regulation and gibberellin signaling and belong to the GRAS family of plant-specific nuclear proteins. They are characterised by the presence of a highly conserved DELLA domain (“DELLA” disclosed as SEQ ID NO: 70) (FIGS. 2d and 11, for example DELLA (SEQ ID NO: 70) or DELLx wherein X is V (SEQ ID NO: 71)) and a SUMOYlation site. In the absence of GA, DELLA proteins repress growth and other GA-dependent processes. In the presence of GA, interaction between the DELLA protein and its receptor induces DELLA degradation. As shown in the examples, SUMOylation represents a novel mechanism of regulating DELLA abundance that is not GA dependent. Both GAI and RGA are SUMOylated in vivo and the SUMOylation site in DELLA proteins is highly conserved (FIGS. 2d and 11). The SUMOylation site in GAI, RGL-2, 3, D8, SLR1, Rht1 and Sln1 is QKLE (SEQ ID NO: 72) (residues 64-67 in GAI). This is located C-terminal of the conserved DELLA site (SEQ ID NO: 70) (residues 44-48 in GAI). As also shown in the examples, site-directed mutagenesis of a SUMOylatable conserved K residue in the SUMOylation site of the DELLA protein RGA abolished SUMOylation.

Thus, in one embodiment of the methods for increasing growth and/or yield and for modulation of SUMOylation, SUMOylation of a DELLA protein selected from RGA-LIKE 1, 2 and 2 (RGL-1, RGL-3 and RGL-2), GIBBERELLIC ACID INSENSITIVE (GAI) or their homologs or orthologues in other plants, including maize D8 (Accession No. NM_{—}001137157, AJ242530), rice SLR1 (Accession No.: AB262980), wheat Rht1 (Accession No.: KC434135), GhSLR (Accession No.: FJ974047) and barley Sln1 ((Accession No.: AK372064) is prevented or decreased. In a preferred embodiment, the DELLA protein is GAI or a GAI homolog or orthologue in other plants, preferably in a crop plant. This can be carried out using the method described above wherein SUMOylation motifs are altered. According to one embodiment of these methods, a nucleic acid encoding a DELLA protein as defined above in which a SUMOylatable residue, for example K, within a SUMOylation motif is deleted or replaced by another, non-SUMOylatable amino acid, for example R, is expressed in a transgenic plant. In one embodiment, one or more residues within the SUMOylation site QKLE (SEQ ID NO: 72) is modified, for example Q, K, L, and/or E.

Thus, in one aspect, the invention relates to a method for modifying growth and/or yield of a plant, preferably under stress conditions, preferably under mild/moderate stress conditions which may comprise expressing a nucleic acid construct in a plant said construct which may comprise a nucleic acid which may comprise SEQ ID NO. 1, 5, 7 or 11 and which encodes a mutant AtRGL-1, AtRGL-2, AtGAI, AtRGL-3 polypeptide, wherein the mutant polypeptide is as defined in SEQ ID No. 2, 6, 8 or 12 or a functional variant homologue or orthologue thereof but which may comprise a substitution of a conserved residue, for example the K residue, in the conserved SUMOylation site. The functional variant homologue or orthologue is not RGA, for example not AtRGA.

According to the various aspects of the invention, growth and/or yield is increased compared to a control plant, plant part or control plant product. The control plant does not express the polynucleotide as described herein. The control plant is preferably a wild type plant. As explained above, in a preferred embodiment, growth is modified under stress, preferably moderate/mild stress.

In one embodiment, the method for increasing growth and/or yield of a plant or part thereof described above further may comprise the steps of screening plants for those that may comprise the polynucleotide construct above and selecting a plant that has an increased growth and/or yield. In another embodiment, further steps include measuring growth and/or yield in said plant progeny, or part thereof and comparing growth and/or yield to that of a control plant.

DELLA proteins have been identified in many plant species, including dicots and monocots. There are a number of DELLA proteins in Arabidopsis, including REPRESSOR OF gal-3 (RGA), RGA-LIKE 1 and 2 (RGL-1 and RGL-2), GIBBERELLIC ACID INSENSITIVE (GAI). The terms “orthologues” and “paralogues” encompass evolutionary concepts used to describe the ancestral relationships of genes. Paralogues are genes within the same species that have originated through duplication of an ancestral gene; orthologues are genes from different organisms that have originated through speciation, and are also derived from a common ancestral gene. Orthologues of the GAI DELLA protein have been described in other plant species, including rice (SLR1), maize (D8, D8-1, D8-MP1, D9), wheat (Rht genes, e.g. Rht-1), barley (SLN) and cotton (GhSLR) (27, 28) (FIG. 11). A skilled person would appreciate that these can be used according to the various aspects of the invention explained herein and the various aspects of the invention specifically relate to these genes and their proteins (for example as shown in FIG. 11).

Thus, based on the various aspects of the invention, the term DELLA protein includes a protein selected from RGL-1 (SEQ ID No. 6), RGL-2 (SEQ ID No. 8), GAI (SEQ ID No. 2), RGL-3 (SEQ ID No. 12), a functional variant homologue or an orthologue thereof, but not RGA. These polypeptides are encoded by the corresponding nucleic acid sequences shown in SEQ ID. Nos. 5, 7, 1 and 11.

The homologue/orthologue of a RGL1-, RGL-2, GAI, RGL-3 polypeptide as defined in SEQ ID No. 2, 6, 8 or 12 has, in increasing order of preference, at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the amino acid represented by SEQ ID NO: 2, 6, 8 or 12. In another embodiment, the homologue/orthologue of a RGL-1, RGL-2, GAI, RGL-3 nucleic acid sequence has, in increasing order of preference, at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the nucleic acid represented by SEQ ID NO: 1, 5, 7 or 11. Preferably, the homologue/orthologue is a GAI homologue/orthologue with at least 80%, 81%, 82%, 83%, 84%, 85%, 86%, 87%, 88%, 89%, 90%, 91%, 92%, 93%, 94%, 95%, 96%, 97%, 98%, or 99% overall sequence identity to the amino acid represented by SEQ ID NO: 2. The overall sequence identity is determined using a global alignment algorithm known in the art, such as the Needleman Wunsch algorithm in the program GAP (GCG Wisconsin Package, Accelrys). A preferred orthologue is selected from D8, SLR1, Rht1 and Sln1 as shown in FIG. 11.

Thus, the nucleotide sequences of the invention and described herein can be used to isolate corresponding sequences from other organisms, particularly other plants, more particularly cereals. In this manner, methods such as PCR, hybridization, and the like can be used to identify such sequences based on their sequence homology to the sequences described herein. Sequences may be isolated based on their sequence identity to the entire sequence or to fragments thereof. In hybridization techniques, all or part of a known nucleotide sequence is used as a probe that selectively hybridizes to other corresponding nucleotide sequences present in a population of cloned genomic DNA fragments or cDNA fragments (i.e., genomic or cDNA libraries) from a chosen plant. The hybridization probes may be genomic DNA fragments, cDNA fragments, RNA fragments, or other oligonucleotides, and may be labeled with a detectable group, or any other detectable marker. Thus, for example, probes for hybridization can be made by labeling synthetic oligonucleotides based on the ABA-associated sequences of the invention. Methods for preparation of probes for hybridization and for construction of cDNA and genomic libraries are generally known in the art and are disclosed in Sambrook, et al., (1989) Molecular Cloning: A Library Manual (2d ed., Cold Spring Harbor Laboratory Press, Plainview, N.Y.).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, preferably less than 500 nucleotides in length. Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Duration of hybridization is generally less than about 24 hours, usually about 4 to 12. Stringent conditions may also be achieved with the addition of destabilizing agents such as formamide.

The term “functional variant of a nucleic acid sequence” as used herein with reference to SEQ ID No. shown herein refers to a variant gene sequence or part of the gene sequence which retains the biological function of the full non-variant sequence, for example confers increased growth or yield when expressed in a transgenic plant. A functional variant also may comprise a variant of the gene of interest which has sequence alterations that do not affect function, for example in non-conserved residues. Also encompassed is a variant that is substantially identical, i.e. has only some sequence variations, for example in non-conserved

In the methods for manipulating growth by modifying the SUMOylation of a DELLA protein selected from RGL-1, 2 or 3, GAI as encoded by SEQ ID NO: 1, 3, 7 or 11 or their homologues or orthologues, growth is modified under abiotic stress conditions. Abiotic stress is preferably selected from drought, salinity, freezing, low temperature or chilling. In one embodiment, the stress is salinity, for example moderate or high salinity. In another embodiment, the stress is drought. Thus, the invention relates to improving growth of a plant under abiotic stress conditions which may comprise altering the SUMOylation status of a DELLA protein selected from RGL-1, 2 or 3, GAI as encoded by SEQ ID NO: 1, 3, 7 or 11 or their homologues or orthologues. This yields plants that show improved growth under stress conditions under which growth of control plants is impaired. Thus, the invention also relates to mitigating the effects of abiotic stress on plant growth by altering the SUMOylation status of a DELLA protein selected from RGL-1, 2 or 3, GAI as encoded by SEQ ID NO: 1, 3, 7 or 11 or their homologues or orthologues. Modification of the SUMOylation site in these methods is as explained below by altering one or more residue in the conserved SUMOylation site.

The stress may be severe or preferably moderate or mild stress. In Arabidopsis research, stress is often assessed under severe conditions that are generally lethal to wild type plants. For example, drought tolerance is assessed predominantly under quite severe conditions in which plant survival is scored after a prolonged period of soil drying. However, in temperate climates, limited water availability rarely causes plant death, but restricts biomass and seed yield. Moderate water stress, that is suboptimal availability of water for growth can occur during intermittent intervals of days or weeks between irrigation events and may limit leaf growth, light interception, photosynthesis and hence yield potential. Leaf growth inhibition by water stress is particularly undesirable during early establishment. There is a need for methods for making plants with increased yield under moderate stress conditions. In other words, whilst plant research in making stress tolerant plants is often directed at identifying plants that show increased stress tolerance under severe conditions that will lead to death of a wild type plant, these plants do not perform well under moderate stress conditions and often show growth reduction which leads to unnecessary yield loss (Skircyz et al, 45).

Thus, in one embodiment of the methods of the invention, yield is improved under moderate or mild stress conditions by altering the SUMOylation status of a gene and expressing the gene in a plant. The transgenic plants according to the various aspects of the invention show enhanced tolerance to these types of stresses compared to a control plant and are able to mitigate any loss in yield/growth. The tolerance can therefore be measured as an increase in yield/growth as shown in the examples and using methods known in the art.

Any given crop achieves its best yield potential at optimal conditions. Mild or moderate stress include any suboptimal environmental conditions, for example, suboptimal water availability or suboptimal temperatures conditions. Moderate or mild stress conditions are well known term in the filed and refer to non-severe stress. Severe stress is generally lethal and leads to the death of a substantial portion of plants. It is generally measured by measuring survival of plants. Moderate or mild stress does not affect plant survival, but it affects plant growth and/or yield. In other words, under mild or moderate (suboptimal) conditions, growth and/or yield of a wild type plant is reduced, for example by at least 10%, for example 10%-50% or more.

The terms moderate or mild stress/stress conditions are used interchangeably and refer to non-severe stress. Severe stress leads to deaths of a significant population of a wild type control population, for example 50-100%, for example at least 50%, at least 60%, at least 70% , at least 80% or at least 90% of the wild type population. In other words, moderate stress, unlike severe stress, does not lead to plant death of the transgenic or the control plant. Under moderate or mild, that is non-lethal, stress conditions, wild type plants are able to survive, but show a decrease in growth and seed production (and thus yield) and prolonged moderate stress can also result in developmental arrest. Tolerance to severe stress is, on the other hand, measured as a percentage of survival, whereas moderate stress does not affect survival, but growth rates. The precise conditions that define moderate stress vary from plant to plant species and also between climate zones, but ultimately, these moderate conditions do not cause the plant to die. With regard to high salinity for example, most plants can tolerate and survive about 4 to 8 dS/m. Specifically, in rice, soil salinity beyond ECe˜4 dS/m is considered moderate salinity while more than 8 dS/m becomes high. Similarly, pH 8.8-9.2 is considered as non-stress while 9.3-9.7 as moderate salinity stress and equal or greater than 9.8 as higher stress.

Drought stress can be measured through leaf water potentials. Generally speaking, moderate drought stress is defined by a water potential of between −1 and −2 Mpa. Moderate temperatures vary from plant to plant and specially between species. Normal temperature growth conditions for Arabidopsis are defined at 22-24° C. For example, at 28° C., Arabidopsis plants grow and survive, but show severe penalties because of “high” temperature stress associated with prolonged exposure to this temperature. The threshold temperature during flowering, which resulted in seed yield losses, was 29.5° C. for all Brassica species. However, the same temperature of 28° C. is optimal for sunflower, a species for which 22° C. or 38° C. causes mild, but not lethal stress. The optimum temperature for growth processes in maize is around 30° C. temperature higher than 30° C. impact on yield/growth.

Suboptimal temperature stress, but not lethal severe stress, can be defined as any reduction in growth or induced metabolic, cellular or tissue injury that results in limitations to the genetically determined yield potential, caused as a direct result of exposure to temperatures below the thermal thresholds for optimal biochemical and physiological activity or morphological development (Greaves et al, 46).

In other words, for each species and genotype, an optimal temperature range can be defined as well as a temperature range that induces mild stress or severe stress which leads to lethality of a significant part of the wild type population.

In another embodiment of the methods for increasing growth of a plant, SUMOylation of the target protein is increased. This can be achieved by introducing additional SUMOylation sites into a target protein and expressing a nucleic acid sequence encoding a mutant target protein in a plant wherein said nucleic acid sequence has been altered in this way to increase SUMOylation of said target protein.

As explained above, the consensus SUMOylation motif is X₁/ΨKX₂E/D. The amino acid sequence of a plant target protein can be altered to introduce one or more SUMOylation sites in addition to any existing SUMOylation sites in the protein. This can be achieved by altering the codons in the corresponding nucleic acid sequence resulting in a peptide which may comprise one or more additional SUMOylation motif. The nucleic acid sequence can be expressed in a transgenic plant using a promoter described herein to increase the amount of target protein that can be SUMOylated. Abundance of SUMOylatable target protein results in an increase in growth.

In one embodiment of these methods of the invention, a mutant nucleic acid is expressed in a transgenic pant which may comprise a modified SUMOylation motif as described above and further may comprise a phosphorylation site downstream of the SUMOylation motif to mediate SUMOylation dependent phosphorylation.

In another aspect, the invention relates to a method for modifying growth and/or yield of a plant which may comprise altering the interaction of a SUMOylated target protein with its receptor. In one embodiment, growth is increased. In one embodiment, this can be achieved by preventing binding of a SUMOylated protein to its receptor. To prevent binding of a SUMOylated protein to its receptor, the binding site of the receptor can be altered for example by site-directed mutagenesis. So-called SUMO-interacting motifs (SIMs) are the mediators of various types of interactions between SUMO and SUMO binding proteins. For example, SIMs form distinct SUMO-binding domains to recognize diverse forms of protein SUMOylation. SIMs have been identified in animals.

Thus, in one embodiment, site-directed mutagenesis of a nucleic acid sequence encoding a receptor protein which binds to a SUMOylated target protein involved in growth regulation is used to change the SIM motif to prevent or decrease binding of the SUMOylated protein to its receptor. The nucleic acid encoding for the mutant amino acid is expressed in a transgenic plant using a promoter described herein.

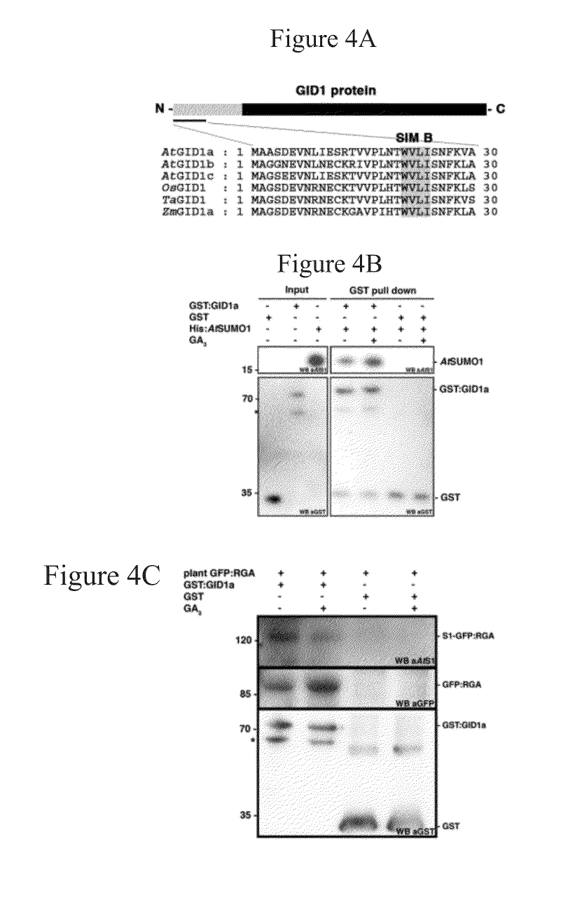

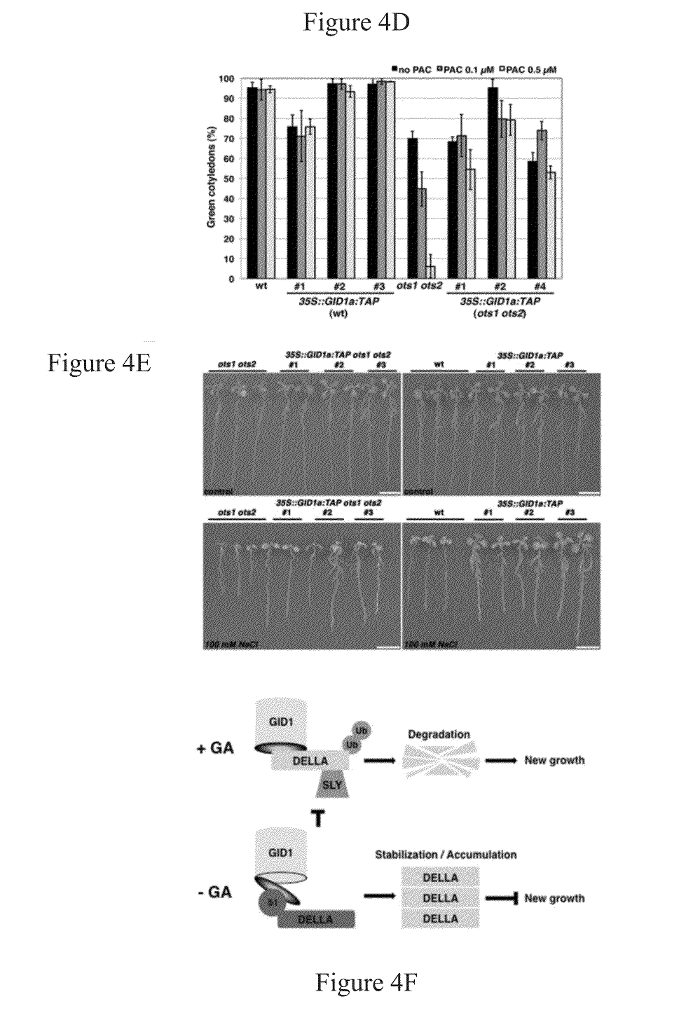

In one embodiment, the target protein is a DELLA protein selected from GAI, RGL-1, 2 or 3 or their homologues or orthologues and the receptor is GID1. In a preferred embodiment, the DELLA protein is selected from GAI, SLR1, D8, D8-1, D8-MP1, D9, Rht, SLN or GhSLR. As shown in the examples, SUMOylation of a DELLA protein mediates binding to the GID1 receptor which is GA independent. The examples also show that GID1 is rate limiting in maintaining the steady state levels of DELLA proteins. SUMOylation of DELLAs then acts as a ‘decoy’ to enhance the levels of non-SUMOylated DELLAs by sequestering the GA receptor GID1 (FIG. 4f). FIG. 17. shows that SUMO inhibits GID1a binding to RGA-DELLA protein.

In Arabidopsis, three GID1 receptors have been identified (AtGID1a, see SEQ ID No. 9 and 10, AtGID1b and AtGID1c). Orthologues of GID1 in other species have also been identified. These include GID1 in maize, wheat, barley, sorghum, and rice (see FIG. 4a). Thus, the GID1 receptor may be Arabidopsis GID1a or a homologue or orthologue thereof. The homologue or orthologue of a AtGID1 polypeptide has, in increasing order of preference, at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the amino acid represented by SEQ ID NO: 10. In one embodiment, the GID1 receptor is ZmGID1 or OsGID1.

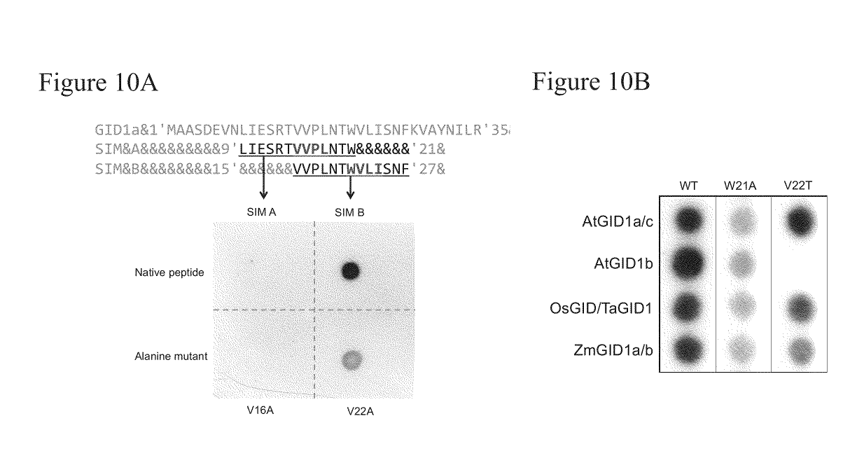

As shown in FIG. 4a, SIM sites are conserved in GID1 polypeptides from different plant species. The core sequence of the SIM site is WVLI (SEQ ID NO: 73). As shown in FIG. 10, peptide array of all SIMs in Arabidopsis, rice and maize show interaction with SUMO1. Moreover, a mutation of the conserved W residue showed reduced interaction with SUMO1 in all GID1 receptors analysed. Thus, creating a mutation in the conserved SIM site of a GID1 protein abolished interaction with SUMO and consequently the SUMOylated target protein. This renders the receptor available for binding to non-SUMOylated DELLA protein and reduces the abundance of non-SUMOylated DELLA. Accordingly, in one aspect, the invention may comprise a method for increasing growth by mutagenesis of a nucleic acid encoding a GID1 receptor wherein one or more codons encoding a SIM motif are altered. In one embodiment, the conserved W and/or V residue in the SIM motif is replaced by another amino acid. As shown in FIG. 14, plants expressing a GID1a receptor in which the SUMOylation site has been altered (35S:GID1a (V22A)) are more resistant to salinity stress and show improved growth under salt stress compared to the wild type. In another embodiment, one or more residues within the SIM site WVLI (SEQ ID NO: 73) are replaced.

Thus, the invention relates to a method for increasing growth and/or yield of a plant under abiotic stress conditions, for example drought or salinity, which may comprise expressing a gene construct encoding a mutant GID1 receptor in a plant wherein the mutation in said receptor prevents binding of a SUMOylated DELLA protein, selected from RGL-1, -2 or -3, GAI as encoded by SEQ ID NO: 1, 3, 7 or 11 or their homologs or orthologues, to its receptor. In one embodiment, the DELLA protein is not RGA. The method may comprise expressing a gene construct encoding a mutant GID1a polypeptide wherein said mutant is as defined in SEQ ID NO: 10 or a functional variant, homolog or ortholog thereof, but may comprise a mutation in the SIM motif. This mutation can be a replacement of one or more residues within the SIM site WVLI (SEQ ID NO: 73), for example W, V, L and/or I or any combination thereof, preferably a substitution of W and/or V. For example, the modification may be V to A and V to S.

In one embodiment, the method for increasing growth and/or yield of a plant or part thereof described above further may comprise the steps of screening plants for those that may comprise the polynucleotide construct above and selecting a plant that has an increased growth and/or yield. In another embodiment, further steps include measuring growth and/or yield in said plant progeny, or part thereof and comparing growth and/or yield to that of a control plant.

In another embodiment, mutagenesis of a nucleic acid sequence encoding a receptor protein which binds to a SUMOylated plant target protein involved in growth regulation is used to change the SIM motif to increase binding of the SUMOylated protein to its receptor.

The altered gene sequences described in the various embodiments of the invention herein can be expressed in the organism using expression vectors commonly known in the art. The mutated sequence may be part of an expression cassette which may comprise a promoter driving expression of said sequence. Said promoter may be the endogenous promoter, a constitutive promoter, or a tissue specific promoter. Using a tissue specific promoter, it is possible to drive expression of the transgene in a tissue specific way thus altering temperature sensing in a particular tissue.

Overexpression using a promoter in plants may be carried out using a constitutive promoter, such as the cauliflower mosaic virus promoter (CaMV35S), the rice actin promoter, the maize ubiquitin promoter, the rice ubiquitin rubi3 promoter or any promoter that gives enhanced expression. Alternatively, enhanced or increased expression can be achieved by using transcription or translation enhancers, introns, or activators and may incorporate enhancers into the gene to further increase expression. Furthermore, an inducible expression system may be used, such as a steroid or ethanol inducible expression system in plants. In one embodiment, the promoter is a plant promoter that is stress promoter, such as the HaHB1 promoter. Other suitable promoters and inducible systems are also known to the skilled person.

As a skilled person will know, the expression may also comprise a selectable marker which facilitates the selection of transformants, such as a marker that confers resistance to antibiotics, for example kanamycin.

Selection of the vector that may comprise the selected sequence of the invention can be carried out by techniques such as:
Selection of cells that contain the vectors of the invention by adding antibiotics to the culture medium. The resistance of these cells to substances such as antibiotics is produced by the synthesis of molecules encoded by a sequence contained in the sequence of the vector.
Digestion with restriction enzymes, by means of which a fragment of some of the sequences of the invention inserted in the vector is obtained.
Detection of a marker gene present in the transformation vector, whose presence in the plant indicates the presence of the sequences of the invention.

The recombinant nucleic acid sequence carrying a mutation as described herein is introduced into a plant and expressed as a transgene. The nucleic acid sequence is introduced into said plant through a process called transformation. The term “introduction” or “transformation” as referred to herein encompasses the transfer of an exogenous polynucleotide into a host cell, irrespective of the method used for transfer. Plant tissue capable of subsequent clonal propagation, whether by organogenesis or embryogenesis, may be transformed with a genetic construct of the present invention and a whole plant regenerated there from. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The polynucleotide may be transiently or stably introduced into a host cell and may be maintained non-integrated, for example, as a plasmid. Alternatively, it may be integrated into the host genome. The resulting transformed plant cell may then be used to regenerate a transformed plant in a manner known to persons skilled in the art. The transfer of foreign genes into the genome of a plant is called transformation. Transformation of plants is now a routine technique in many species. Advantageously, any of several transformation methods may be used to introduce the gene of interest into a suitable ancestor cell. The methods described for the transformation and regeneration of plants from plant tissues or plant cells may be utilized for transient or for stable transformation. Transformation methods include the use of liposomes, electroporation, chemicals that increase free DNA uptake, injection of the DNA directly into the plant, particle gun bombardment, transformation using viruses or pollen and microprojection. Methods may be selected from the calcium/polyethylene glycol method for protoplasts, electroporation of protoplasts, microinjection into plant material, DNA or RNA-coated particle bombardment, infection with (non-integrative) viruses and the like. Transgenic plants, including transgenic crop plants, are preferably produced via Agrobacterium tumefaciens mediated transformation.

Thus, the invention relates to a method for producing a transgenic plant with improved with improved yield/growth under stress conditions said method which may comprise
a) introducing into said plant and expressing a nucleic acid encoding an altered DELLA protein selected from GAI, RGL-1, -2 or -3 or a homolog or ortholog thereof for example SLR1, D8, D8-1, D8-MP1, D9, Rht, SLN or GhSLR wherein the SUMOylation site is altered as described above and
b) obtaining a progeny plant derived from the plant or plant cell of step a).

Thus, the invention relates to a method for producing a transgenic plant with improved with improved yield/growth under stress conditions said method which may comprise
a) introducing encoding a mutant GID1 receptor in a plant wherein the mutation in said receptor prevents binding of a SUMOylated DELLA protein, selected from RGL1, 2 or 3, GAI as encoded by SEQ ID NO: 1, 3, 7 or 11 or a homolog or ortholog thereof, to its receptor and
b) obtaining a progeny plant derived from the plant or plant cell of step a).

The method may comprise expressing a gene construct encoding a mutant GID1a polypeptide wherein said mutant is as defined in SEQ ID NO: 10 or a functional variant, homolog or ortholog thereof, but may comprise a mutation in the SIM motif. This mutation can be a replacement of one or more residues within the SIM site WVLI (SEQ ID NO: 73), for example W, V, L and or I or any combination thereof, preferably a substitution of W and/or V. For example, the modification may be V to A and V to S.

The invention also provides a transgenic plant obtained or obtainable by the methods described herein. In one embodiment, the plant expresses a nucleic acid sequence encoding an altered DELLA protein selected from GAI, RGL-1, 2 or 3 or their homologs or orthologues for example SLR1, D8, D8-1, D8-MP1, D9, Rht, SLN or GhSLR wherein the SUMOylation site is altered as described above. In another embodiment, the plant expresses an altered DELLA receptor, for example GID1a.

Furthermore, the invention also provides a method for improving stress tolerance, for example abiotic stress. In one embodiment, the stress is high or moderate salinity. In another embodiment, the stress is drought. As described in the examples, sequestration of GID1 by SUMO-conjugated DELLAs leads to an accumulation of non-SUMOylated DELLAs and subsequent growth restraint during stress. Thus, reducing the abundance of non-SUMOylated DELLAs increases growth. As described above, this can be achieved by preventing SUMOylation of the target protein thus rendering the GID1 receptor available to non-SUMOylated DELLAs. This can be achieved by altering the SUMOylation motif of the target protein as described above. The target protein is not limited to DELLA proteins and any protein involved in growth regulation can be used. In one embodiment, the protein is a DELLA protein. In another embodiment, the interaction of the target protein with the receptor is altered, for example by removing or altering the SIM motif in the receptor to prevent binding of SUMOylated protein to the receptor. Thus, the invention relates to a method for improving stress tolerance to abiotic stress which may comprise expressing a gene construct in a plant encoding for a DELLA protein selected from GAI, RGL-1, 2 or 3 or their homologs or orthologues as defined in SEQ ID No. 2, 6, 8 or 12 and in FIG. 11 wherein the SUMOylation site in said DELLA protein has been altered to prevent SUMOylation. As explained herein, the SUMOylation site can be altered by substitution of the conserved K residue in the DELLA protein SUMOylation site. In another embodiment, of the method for improving stress tolerance to abiotic stress may comprise expressing a gene construct in a plant encoding for a GID1a receptor or a homolog or orthologue thereof in which the SUMOylation site of the receptor has been altered. As explained herein, the SUMOylation site can be altered by substitution of the conserved W or V residue in the receptor SIM site. For example, the modification may be V to A and V to S.

In a preferred embodiment of the method, the DELLA protein is selected from GAI, SLR1, D8, D8-1, D8-MP1, D9, Rht1, SLN or GhSLR and the stress is moderate or high salinity or moderate or high drought. Accession numbers for these genes are given elsewhere herein and sequences can thus be readily identified by the skilled person. Applicants also refer to the peptide sequence

The invention also provides a method of preventing SUMOylation of a plant protein involved in growth regulation. As described above, this can be achieved by substituting or deleting one or more residue in the conserved SUMOylation site, preferably the K residue.

The invention also provides an isolated nucleic acid encoding for a plant protein for example involved in growth regulation in which one or more SUMOylation sites have been modified. In one embodiment, some or all SUMOylatable conserved K residues have been replaced by non-SUMOylatable residues. In one embodiment, the modified protein is a DELLA protein as described herein. Thus, the isolated nucleic acid encodes for a DELLA selected from GAI, RGL-1, 2 or 3 or their homologues or orthologues as defined in SEQ ID No. 2, 6, 8 or 12 but which may comprise a substitution of one or more conserved residue, for example K, in the conserved SUMOylation site (as shown in FIGS. 2d and 11). Thus, the naturally occurring nucleic acid has been altered by human intervention to introduce specific mutations in the target SUMOylation site. In one embodiment, the nucleic acid is cDNA. The invention also provides an expression vector which may comprise such a nucleic acid. In another aspect, the invention relates to an isolated host plant or bacterial cell, for example Agrobacterium tumefaciens cell, transformed with a vector or a nucleic acid sequence as described above. The cell may be comprised in a culture medium. Thus, in one aspect the invention also relates to a culture medium which may comprise an isolated host plant cell transformed with a vector or a nucleic acid sequence in which one or more SUMOylation sites have been modified as described above.

The invention also provides the use of an isolated nucleic acid sequence or molecule or expression vector described above in methods for increasing growth.

The invention further provides a transgenic plant expressing a nucleic acid sequence encoding for a protein in which one or more SUMOylation sites have been modified as described herein. In one embodiment, the protein is a DELLA protein selected from GAI, RGL-1, 2 or 3 or their homologues or orthologues as described herein. Thus, in one embodiment, the plant expresses a nucleic acid construct which may comprise a nucleic acid that encodes for a DELLA selected from GAI, RGL-1, 2 or 3 as encoded by SEQ ID NO: 1, 3, 7 or 11 or their homologues or orthologues as defined in SEQ ID No. 2, 6, 8 or 12 but which may comprise a substitution of one or more conserved residue, for example K, in the conserved SUMOylation site (as shown in FIGS. 2d and 11). GAI orthologues selected from D8, Rht1, SLR1 and Sln1 are preferred.

The plant is characterised by increased growth under stress conditions, for example high or moderate salinity or drought.

The invention also provides an isolated nucleic acid encoding for a plant receptor protein involved in growth regulation in which one or more SIM sites have been modified as described herein to decrease, prevent or increase binding of a SUMOylated target protein to its receptor. In one embodiment, the target protein is a DELLA protein as described herein which binds to a GID1 receptor. Thus, the isolated nucleic acid encodes a GID1a receptor as defined in SEQ ID No. 10 but which may comprise a substitution or one or more residue within the SIM site, for example of the conserved W or V residue or the K residue (as shown in FIG. 4a). For example, the modification may be V to A and V to S.

The invention also provides an expression vector which may comprise such a nucleic acid. In another aspect, the invention relates to an isolated plant or bacterial, for example Agrobacterium tumefaciens, host cell transformed with a vector or a nucleic sequence as described above. The cell may be comprised in a culture medium. Thus, in one aspect the invention also relates to a culture medium which may comprise an isolated host plant cell transformed with a vector or a nucleic acid sequence in which one or more SIM sites have been modified as described above.

The invention also provides the use of an isolated nucleic acid or an expression vector as described above in methods for increasing growth or stress tolerance, for example to drought or salinity.

The invention further provides a transgenic plant expressing a nucleic acid encoding for a protein in which one or more SIM sites have been modified. In one embodiment, the protein is a DELLA protein receptor as described herein. Thus, in one embodiment, the plant expresses a nucleic acid construct which may comprise a nucleic acid that encodes a GID1a receptor as defined in SEQ ID No. 10 but which may comprise a substitution of one or more residue within the SIM site, for example of the conserved W or V residue or the K residue in the conserved SUMOylation site (as shown in FIG. 4a).

The invention also relates to a method for producing a transgenic plant with improved with improved yield/growth under stress conditions said method which may comprise
a) introducing into said plant and expressing a nucleic acid construct which may comprise a nucleic acid that encodes a GID1a receptor as defined in SEQ ID No. 10 or a homolog or ortholog thereof but which may comprise a substitution of one or more residue within the SIM site, for example of the conserved W or V residue or the K residue in the conserved SUMOylation site and
b) obtaining a progeny plant derived from the plant or plant cell of step a).

In another embodiment of the methods of the invention for increasing growth of a plant by decreasing or preventing SUMOylation, the decrease or prevention of SUMOylation is achieved by targeting other components of the SUMOylation pathway that interact with the target protein.

For example, inhibiting SUMO proteases using cysteine protease inhibitors prevents SUMOylation of the target protein. Furthermore, agents that block SIM or SUMO sites prevent binding or SUMOylation itself or binding of the target protein to the SIM motif in the receptor.

The invention therefore also provides an in vitro or in vivo assay for identifying a target compound that reduces or prevents SUMOylation of a protein in a plant. The compound may be an agonist or antagonist of the SUMOylation pathway. In one embodiment, the compound is a cysteine protease inhibitor. In another embodiment, the compound is a compound that blocks SIM or SUMO sites to prevent binding or SUMOylation itself or binding of the target protein to the SIM motif in the receptor.

In another embodiment of the methods of the invention for increasing growth or root development of a plant by increasing SUMOylation, the increase of SUMOylation is achieved by targeting other components of the SUMOylation pathway that interact with the target protein. For example, allosteric potentiators (activators of SUMO proteases) can be used.

The invention therefore also provides an in vitro or in vivo assay for identifying a target compound that increases SUMOylation of a protein in a plant. In one embodiment, the compound is an activator of SUMO proteases. In another embodiment, the compound is a compound that increases SUMOylation itself or increases the binding of the target protein to the SIM motif in the receptor.

In another aspect, the invention provides a method for identifying a compound that regulates, that is increases, decreases or prevents SUMOylation.

These assays can be used to identify compounds that bind to target SUMO sites or prevent SUMO ligases from binding to plant target proteins and therefore block SUMOylation. Conversely there could be chemicals that enhance SUMO E3 binding to targets and hence increase SUMOylation.

In another aspect, the invention relates to compounds identified by the methods above.

In a further aspect, the invention relates to methods using compounds, for example compounds identified by the methods above, in altering the SUMOylation status of the plant target protein by interfering with the SUMOylation pathway. The method may comprise treating a plant with a chemical compound or expressing in a plant a gene encoding a compound that alters the SUMOylation status of the target protein.

Also within the scope of the invention is altering growth of a plant by altering a component, or components, involved in the SUMOylation pathway and which directly or indirectly interact with the target protein, such as SUMO proteases. Thus, expression of SUMO proteases may be upregulated, for example by introducing a construct which may comprise a nucleic acid encoding for a SUMO protease in a plant and expressing said one or more SUMO protease in the plant. In another aspect, expression of SUMO proteases may be downregulated, for example using RNAi technology.

Finally, the invention relates to methods for improving seed vigour by modifying the SUMOylation status of a germination regulator, preferably a DELLA protein or its interaction with its receptor, and also for detecting the SUMOylation status of a germination regulator, preferably a DELLA protein, in a seed, or the status of its interaction with its receptor, and thereby inferring the vigour of that seed, or that of its peers. The germination regulator is selected from a DELLA protein, DOG1, PIL5, SPT, PYR1, ABI5 or COMATOSE. In a preferred method, the regulator is a DELLA protein. In these methods, seeds are analysed to determine the SUMOylation status of a DELLA protein, for example by using anti-SUMO antibodies for the detection of SUMOylated DELLA protein. Using specific anti-SUMO antibodies, the level of SUMOylated DELLA protein can be identified in immunoblot studies using total protein extracts. In addition, protein extraction buffers containing proteasome inhibitors and SUMO protease inhibitors can be utilised to generate a SUMO protein modification profile of each of the targets using a combination of immunoprecipitation and Western blotting techniques.

Thus, in a further step of the method the patterns for target protein stability and also a protein modification profile for each of the targets are obtained. In a further step, the see vigour is determined on the basis of the patterns for target protein stability and also a protein modification profile for each of the targets.

In one embodiment, additional germination regulators, for example DOG1, PIL5, SPT, PYR1, ABI5 or COMATOSE are also analysed. Furthermore, additional post transcriptional mechanisms, such as ubiquitination and phosphorylation can also be analysed in embodiments of this method.

High seed vigour is the cornerstone of sustainable crop production as it greatly influences the number of seedlings that emerge as well as timing and uniformity of emergence. This has a direct crop-specific influence on marketable yield in agriculture and horticulture. In addition, poor emergence has an environmental impact, because chemical inputs (pesticides, herbicides, fertilisers), irrigation and land are not used efficiently; therefore input costs (financial and environmental) remain the same or higher, while marketable yield is reduced. Residual dormancy is the major factor affecting seed quality and despite considerable breeding efforts of selecting for increased seed/seedling vigour, it remains a major problem for industry. It is estimated that between 30-80% of harvested seed in seed production fields is not marketable because of poor quality. The lack of robust tools for confidently predicting seed vigour in the field further adds to the loss of marketable seed to breeders and crop yield to growers.

DELLA proteins are involved in germination. Modifying the SUMOylation status of a DELLA protein can improve seed vigour. Seed vigour may be measured by percentage germination. Furthermore, altering the binding of SUMOylated DELLA protein to their receptor can also improve seed vigour.

In a further aspect, the invention relates to methods for decreasing growth by altering the SUMOylation status of a target protein. The SUMOylation may be increased or decreased using the methods described herein. In yet a further aspect, the invention relates to methods for decreasing growth by altering SUMOylation sites of a receptor as described herein. In one embodiment, the target protein is a DELLA protein. The invention also relates to transgenic plants obtained through such methods, related uses and methods for repressing growth by altering the SUMOylation status of a target protein.

The terms “decrease”, “reduce” or are interchangeable. Growth is decreased by at least a 3%, 4%, 5%, 6%, 7%, 8%, 9% or 10%, preferably at least 15% or 20%, more preferably 25%, 30%, 35%, 40% or 50% or more in comparison to a control plant. Growth can be measured for example by measuring hypocotyl or stem length.

In another aspect of the methods described herein, the target protein is selected from ARF19 or ARF7. As explained above, these proteins are regulators of root architecture and play a key role in regulating root architecture. In particular, these proteins can direct the formation of tap root formation v. lateral root formation. Accordingly, by manipulating these proteins to change their SUMOylation state root architecture can be altered in different ways in transgenic plants expressing modified ARF19 or ARF7 proteins.

There are two main types of root according to origin of development and branching pattern in the angiosperms: taproot system and fibrous system. Generally, plants with a taproot system are deep-rooted in comparison with plants having fibrous roots. The taproot system enables the plant to anchor better to the soil and obtain water from deeper sources. In contrast, shallow-rooted plants are more susceptible to drought but they have the ability to respond quickly to fertilizer application. In grasses and other monocots including rice and cereals, the root system is a fibrous root system consisting of a dense mass of slender, adventitious roots that arise from the stem. A fibrous root system has no single large taproot because the embryonic root dies back when the plant is still young. The roots grow downward and outward from the stem, branching repeatedly to form a mass of fine roots.

Plant roots are essential to facilitate the uptake of nutrients and improving root architecture, such as increasing the formation of lateral roots, is particularly beneficial under stress conditions and to improve response to fertiliser and poor soil conditions. On the other hand, increasing the formation of a deep tap root system can be used to increase drought resistance.

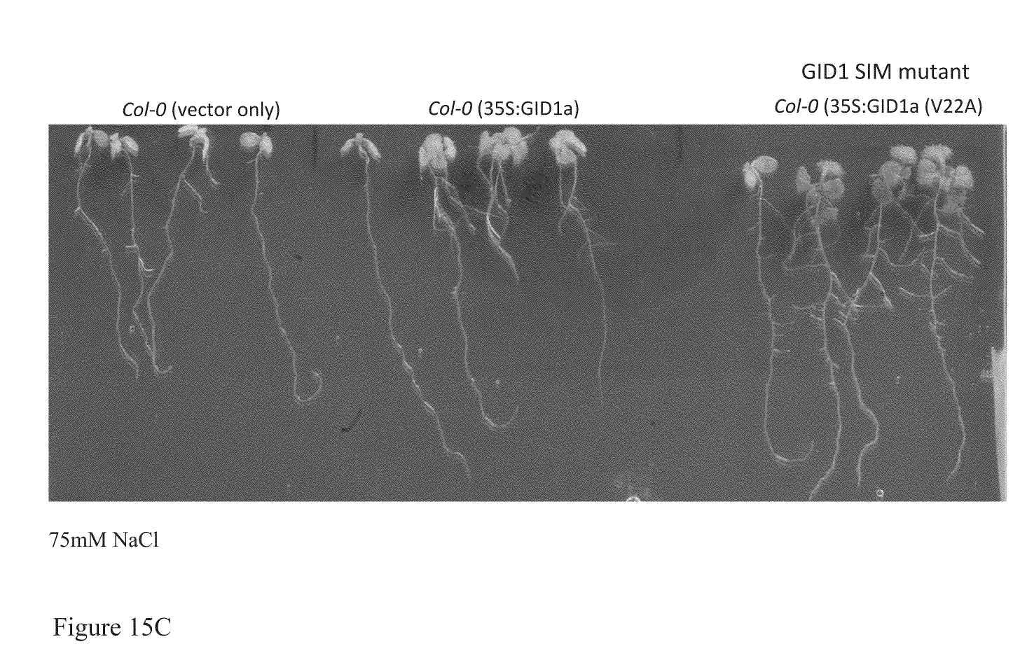

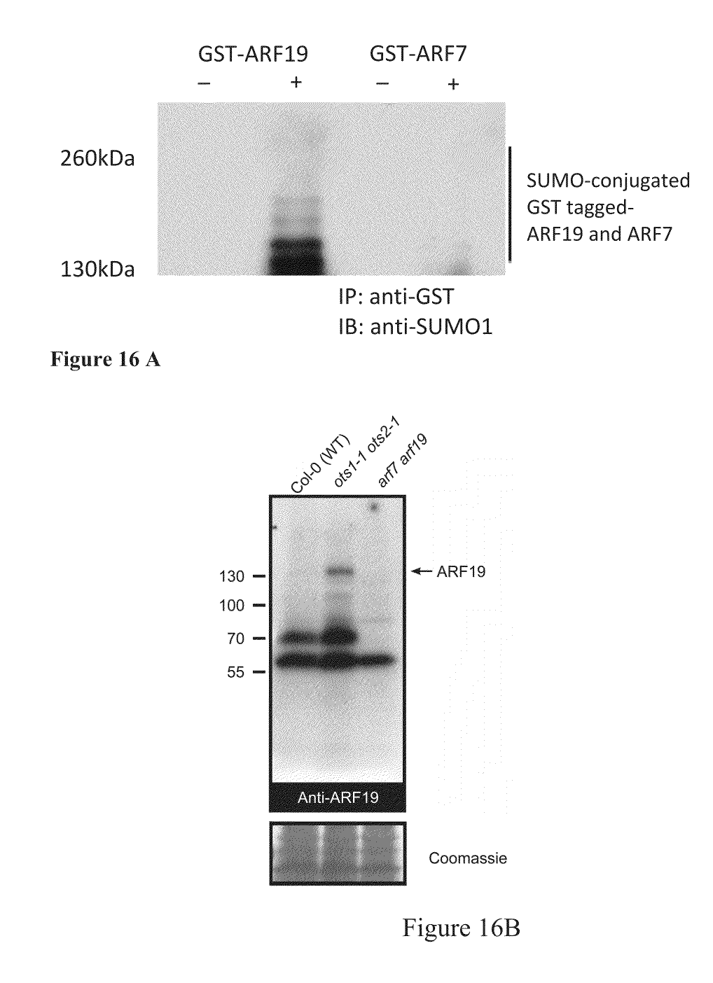

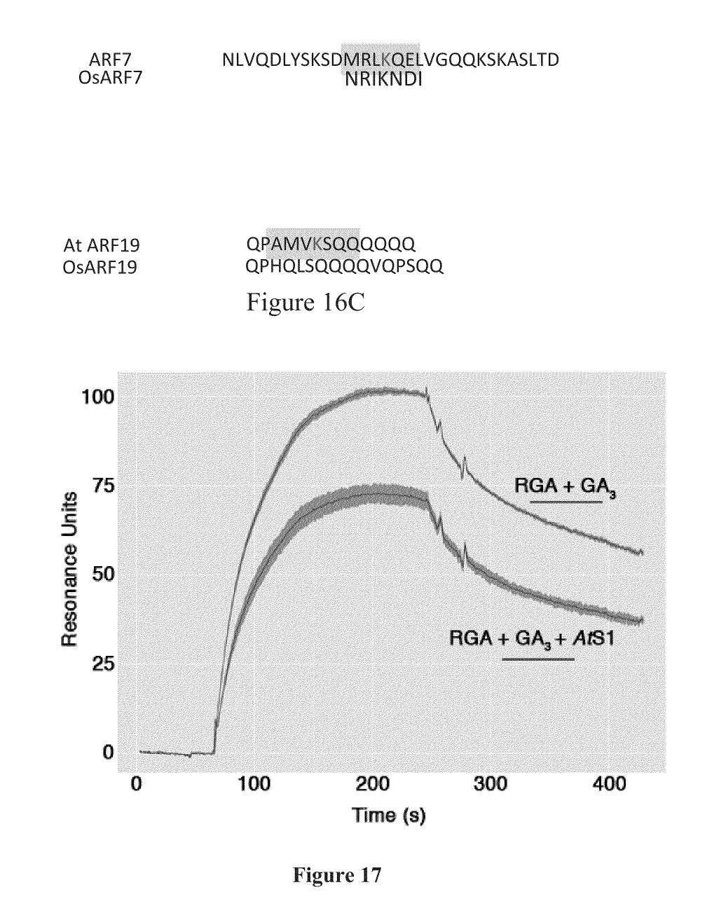

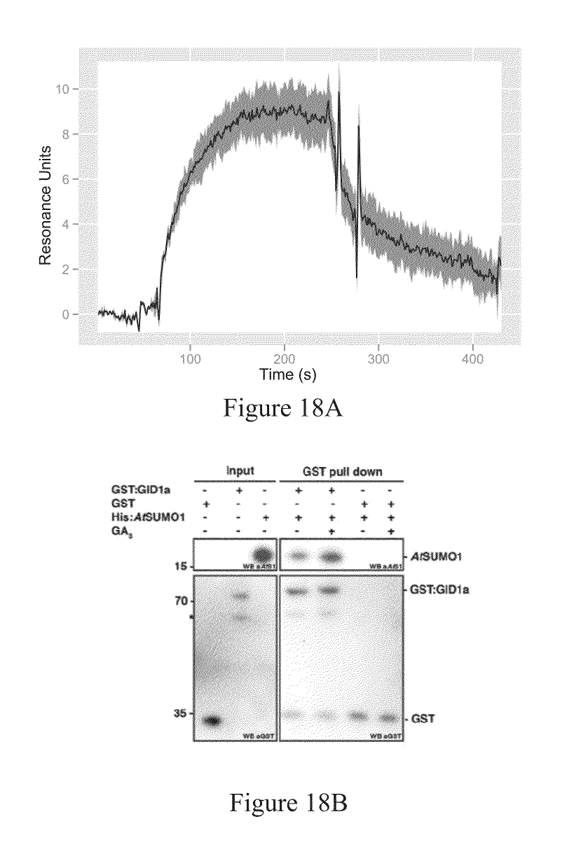

The inventors have demonstrated that AtARF19 and AtARF7 are SUMOylated and they have identified SUMOylation sites in the AtARF19 and AtARF7 proteins (FIG. 16). The inventors have also shown that AtARF19 protein levels are upregulated in ots1/2 SUMO protease mutants. In other words, the absence of SUMO protease increases the presence of the protein as tit is no longer the target of the SUMO protease. Thus, it is clear that AtARF19 and AtARF7 are SUMOylated and that SUMOylation has an effect on the AtARF19 and AtARF7 protein and/or their gene expression. Furthermore, the inventors have also shown that in OsARF19/7, the SUMOylation sites that can be found in AtARF19 and AtARF7 are missing. As explained above, rice has, like other cereals, a branched root system with many lateral roots. Accordingly, the inventors postulate that in the absence of SUMOylation of OsARF19/7 due to missing SUMOylation sites, the formation of a fibrous root system is favoured. Thus, preventing SUMOylation of ARF19/7, preferably in plants that have a tap root system (non-cerals), leads to the formation of more lateral roots compared to control plants and a root phenotype that is more akin to what can be observed in cereals.

On the other hand, increasing SUMOylation of ARF19/7 leads to an improved tap root system compared to control plants.

Thus, in another aspect, the invention relates to a method for altering root architecture by manipulating SUMOylation of a AtARF19 or AtARF7 polypeptide as defined in SEQ ID No. 14 or 16, a functional variant, homolog or ortholog thereof.

In one embodiment, the invention relates to a method for increasing the formation of lateral roots which may comprise preventing or decreasing SUMOylation of AtARF19 or AtARF7 as defined in SEQ ID No. 14 or 16, a functional variant, homolog or ortholog thereof. According to this method, a mutant of AtARF19 or AtARF7 as defined in SEQ ID No. 14 or 16, a functional variant, homolog or ortholog thereof which may comprise an altered SUMOylation site is introduced and expressed into a plant by recombinant methods. The transgenic plants expressing the mutant protein show more lateral root formation compared to control plants which do not express said mutant protein. The plant is preferably a dicot plant.

The protein can be modified using the methods described above wherein the SUMOylation motif in the protein is altered to remove the SUMOylation site thus preventing or decreasing SUMOylation of the protein. According to one embodiment of these methods, a nucleic acid encoding AtARF19 or AtARF7, a functional variant, homologue or orthologue thereof in which one or more SUMOylatable residue within the SUMOylation motif, for example K, is deleted or replaced by another, non-SUMOylatable amino acid, for example R, is expressed in a transgenic plant. The SUMOylation site in ARF7 is MRLKQEL (SEQ ID NO: 74) and in ARF19 AMVKSQQ (SEQ ID NO: 75) (see FIG. 16c). K in the SUMOylation motif is a preferred target and this may be combined with other modifications in the motif. Also, aside from K, any conserved residue in the motif may be altered. Thus, for ARF7, one or more of M, R, L, K, Q, E and/or L can be altered. For ARF19, one or more of A, M, V, K, S, Q and/or Q can be altered.

In one embodiment, the invention relates to a method for improving the formation of a tap root system which may comprise increasing SUMOylation of a AtARF19 or AtARF7 polypeptide as encoded by SEQ ID No. 14 or 16, a functional variant, homolog or ortholog thereof. According to this method, a mutant AtARF19 or AtARF7 as defined in SEQ ID No. 14 or 16, a functional variant, homolog or ortholog thereof but which may comprise additional SUMOylation sites as defined above is introduced and expressed into a plant by recombinant methods. The transgenic plants expressing the mutant protein shows an improved tap root system compared to control plants which do not express said mutant protein. The plant is a dicot or monocot plant as defined herein. Crop plants, for example dicot crop plants, are preferred.

The invention also provides an isolated nucleic acid encoding for AtARF19 or AtARF7, a functional variant, homologue or orthologue thereof in which one or more SUMOylation sites have been modified. In one embodiment, one or more conserved SUMOylatable conserved residues have been replaced by non-SUMOylatable residues. In one embodiment, K has been replaced. In one embodiment, for ARF7, one or more of M, R, L, K, Q, E and/or L can be altered. For ARF19, one or more of A, M, V, K, S, Q and/or Q can be altered. Thus, the naturally occurring nucleic acid has been altered by human intervention. In one embodiment, the nucleic acid may be cDNA.

Thus, the isolated nucleic acid as defined in SEQ ID No. 13 or 15 encodes for AtARF19 or AtARF7 as defined in SEQ ID No. 14 or 16 or a functional variant, homolog or ortholog thereof but which may comprise a substitution of one or more residue, for example of the K residue, in the conserved SUMOylation site. The invention also provides an expression vector which may comprise such a nucleic acid. In another aspect, the invention relates to an isolated host plant or bacterial cell, for a example Agrobacterium tumefaciens cell, transformed with a vector or a nucleic acid sequence as described above. The cell may be comprised in a culture medium. Thus, in one aspect the invention also relates to a culture medium which may comprise an isolated host plant cell transformed with a vector or a nucleic acid sequence in which one or more SUMOylation sites have been modified as described above.

The invention also provides the use of an isolated nucleic acid sequence as defined in SEQ ID No. 13 or 15 that encodes for a AtARF19 or AtARF7 polypeptide as defined in SEQ ID No. 14 or 16 or a functional variant, homologue or orthologue thereof but which may comprise a substitution of one or more conserved residue, for example the K residue, in the conserved SUMOylation site or the use of an expression vector which may comprise said nucleic acid in methods for manipulating root architecture, for example to increase the formation of lateral roots. The invention also provides the use of an isolated nucleic acid sequence as defined in SEQ ID No. 13 or 15 that encodes for a AtARF19 or AtARF7 polypeptide as defined in SEQ ID No. 14 or 16 or a functional variant, homologue or orthologue thereof but which may comprise additional SUMOylation or the use of an expression vector which may comprise said nucleic acid to improve the tap root system.

The invention further provides a transgenic plant expressing a nucleic acid sequence encoding for a AtARF19 or AtARF7 polypeptide as defined in SEQ ID No. 14 or 16 or a functional variant, homolog or ortholog in which one or more SUMOylation sites have been modified as described herein or which may comprise an increased number of SUMOylation sites. Thus, the plant expresses a construct which may comprise a nucleic acid as defined in SEQ ID No. 13 or 15 that encodes for a AtARF19 or AtARF7 polypeptide as defined in SEQ ID No. 14 or 16 or a functional variant, homologue or orthologue thereof but which may comprise a substitution of, for example, the K residue in the conserved SUMOylation site (as shown in FIG. 16).

The invention also provides a method of producing a plant with an altered root phenotype, preferably increased lateral root formation which may comprise incorporating a nucleic acid as defined in SEQ ID No. 13 or 15 encodes for a AtARF19 or AtARF7 polypeptide as defined in SEQ ID No. 14 or 16 or a functional variant, homologue or orthologue thereof but which may comprise a substitution of, for example, the K residue in the conserved SUMOylation site into a plant cell by means of transformation, and; regenerating the plant from one or more transformed cells. Another aspect of the invention provides a plant produced by a method described herein which displays altered root development relative to controls.

The invention also relates to a method for increasing tolerance of a plant to nutrient-deficient conditions, which may comprise incorporating a nucleic acid as defined in SEQ ID No. 13 or 15 encodes for a AtARF19 or AtARF7 polypeptide as defined in SEQ ID No. 14 or 16 or a functional variant, homologue or orthologue thereof but which may comprise a substitution of, for example, the K residue in the conserved SUMOylation site into a plant cell by means of transformation, and; regenerating the plant from one or more transformed cells.

The invention also relates to a method for increasing tolerance of a plant to water deficit conditions, which may comprise incorporating a nucleic acid as defined in SEQ ID No. 13 or 15 encodes for a AtARF19 or AtARF7 polypeptide as defined in SEQ ID No. 14 or 16 or a functional variant, homologue or orthologue thereof but which may comprise additional SUMOylation sites into a plant cell by means of transformation, and; regenerating the plant from one or more transformed cells.

Preferably, the aspects relating to ARF7 and ARF19 relate to manipulation of dicot plants to increase lateral root formation.

Methods that solely rely on conventional breeding techniques and do not involve recombinant technologies are disclaimed.

It will be understood by the skilled person that the transgene is preferably stably integrated into the transgenic plants described herein and passed on to successive generations. A skilled person will also understand that the target genes identified herein and which are expressed in a plant according to the various methods of the invention are expressed as transgenes using recombinant methods. For example, the nuclei acid as used in these methods is part of a heterologous gene expression construct which may comprise the nucleic acid and a regulatory sequence driving expression of said sequence. Plants identified as having a stable copy of the transgene may be sexually or asexually propagated or grown to produce off-spring or descendants. “Heterologous” indicates that the gene/sequence of nucleotides in question or a sequence regulating the gene/sequence in question, has been linked to the target nucleic acid using genetic engineering or recombinant means, i.e. by human intervention. “Isolated” indicate that the isolated molecule (e.g. polypeptide or nucleic acid) exists in an environment which is distinct from the environment in which it occurs in nature. For example, an isolated nucleic acid may be substantially isolated with respect to the genomic environment in which it naturally occurs.

All references mentioned herein are incorporated by reference. Other objects and advantages of this invention will be appreciated from a review of the complete disclosure provided herein and the appended claims.

While the present invention has been generally described above, the following non limiting examples are provided to further describe the present invention, its best mode and to assist in enabling those skilled in the art to practice this invention to its full scope. The specifics of these examples should not be treated as limiting, however.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### EXAMPLES

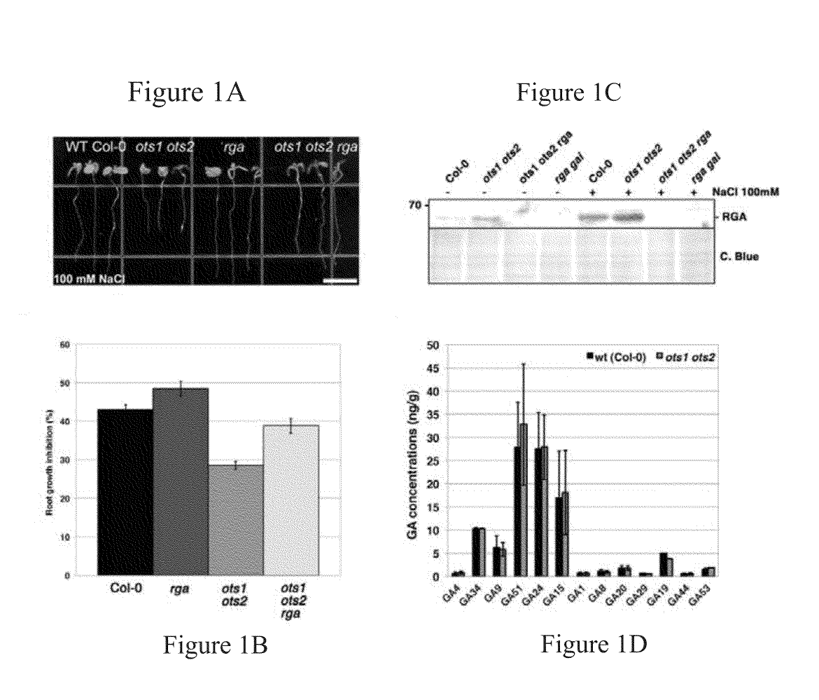

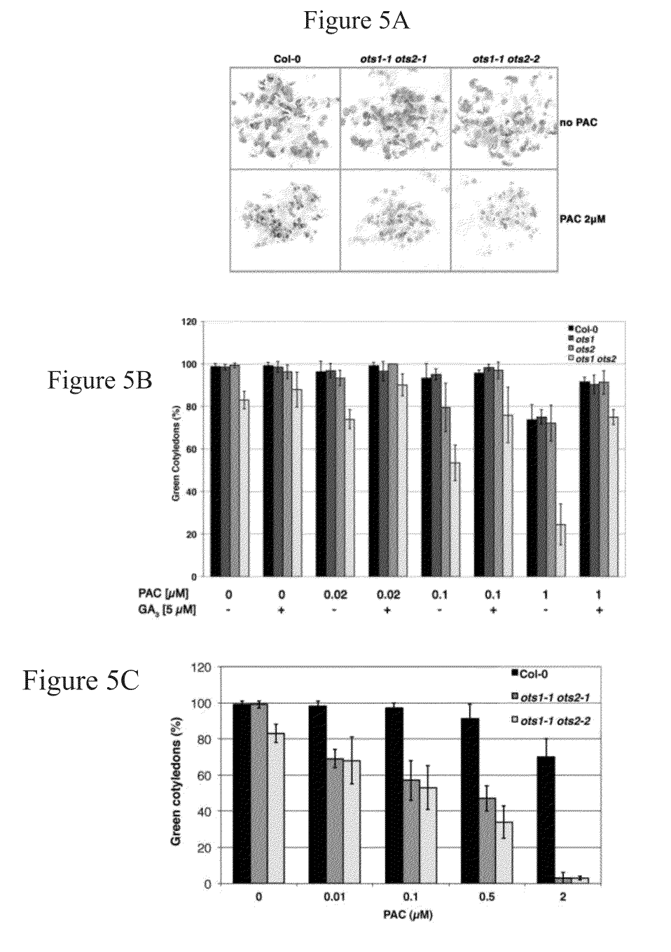

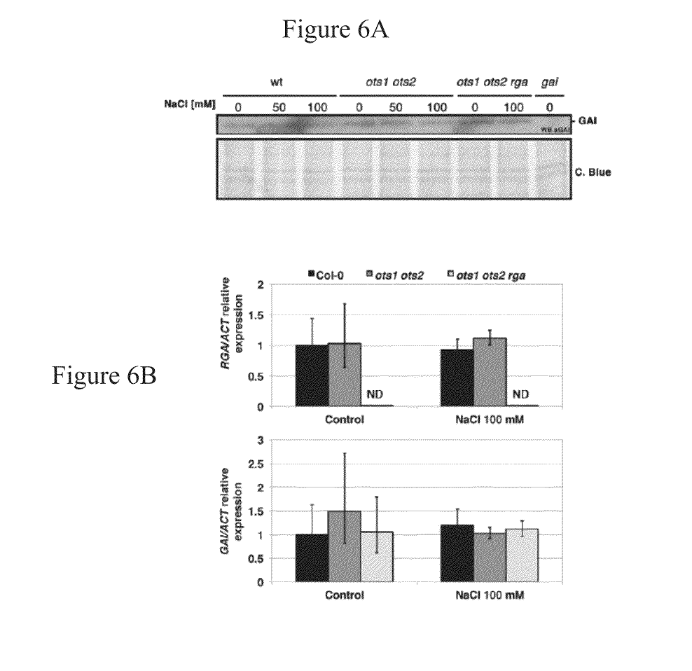

Several ubiquitin-like proteins have been described in plants including SUMO that can act to stabilize the proteins with which it is conjugated (29). SUMO proteases remove SUMO to destabilize the de-conjugated protein (30). Arabidopsis mutant seedlings lacking the SUMO proteases OTS1 and OTS2 exhibit inhibition of root growth when exposed to a 100 mM salt stress (31) (FIG. 1a). Applicants addressed whether DELLAs contribute to the reduced growth phenotype of ots1 ots2 in the presence of salt by creating an ots1 ots2 rga triple mutant, which lacks the RGA DELLA protein. Indeed loss of RGA function was sufficient to alleviate the reduced root growth phenotype of ots1 ots2 double mutant on this permissive concentration of NaCl (FIG. 1a, b). Further observations confirmed that ots1 ots2 plants were affected in not only RGA function but also that of other DELLAs, including those with more specialized functions (e.g. RGL2 which controls seed germination 32, 33), (FIG. 5a, b, c). Hence, the ots1 ots2 mutant reveals a novel link between SUMOylation and DELLA-mediated growth regulation. To directly assess the impact of the ots1 ots2 mutations on DELLA protein abundance, immunoblot experiments were performed. This revealed that endogenous levels of RGA and GAI DELLA proteins were more abundant in the ots1 ots2 mutant plants compared to wild type (FIG. 1c and FIG. 6a). Moreover, this effect was even more pronounced when plants are grown on salt-containing medium (FIG. 1c). The current model for GA signaling dictates that regulation of the abundance of DELLA proteins is directly related to changes in levels of GA. However, Applicants observed that there were no significant differences in GA levels between ots1 ots2 mutant and wild type plants (FIG. 1d). Real-time quantitative RT-PCR also failed to detect a significant difference in RGA and GAI transcript levels in the ots1 ots2 mutant in the presence or absence of 100 mM NaCl (FIG. 6b). Since increased DELLA gene transcription or altered GA accumulation could not account for the increased DELLA protein accumulation observed in ots1 ots2 mutants, Applicants hypothesized that it could be caused by a novel GA independent posttranslational mechanism.

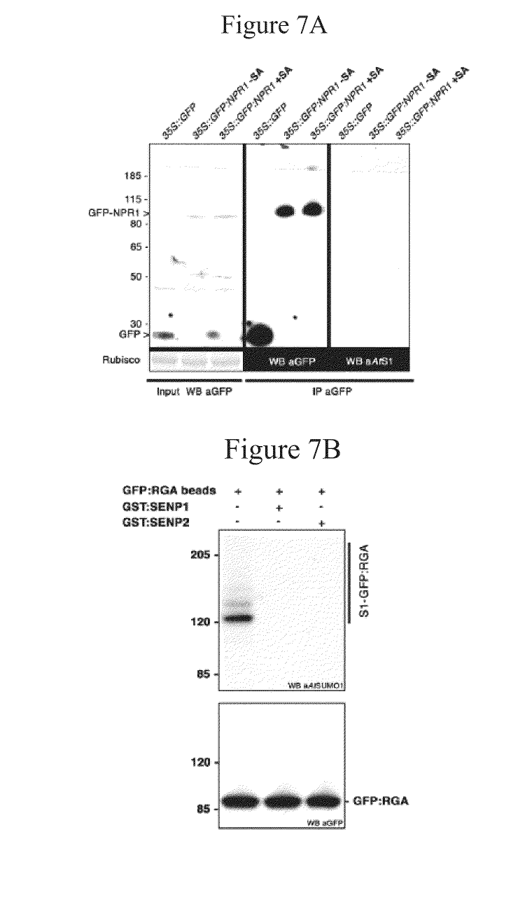

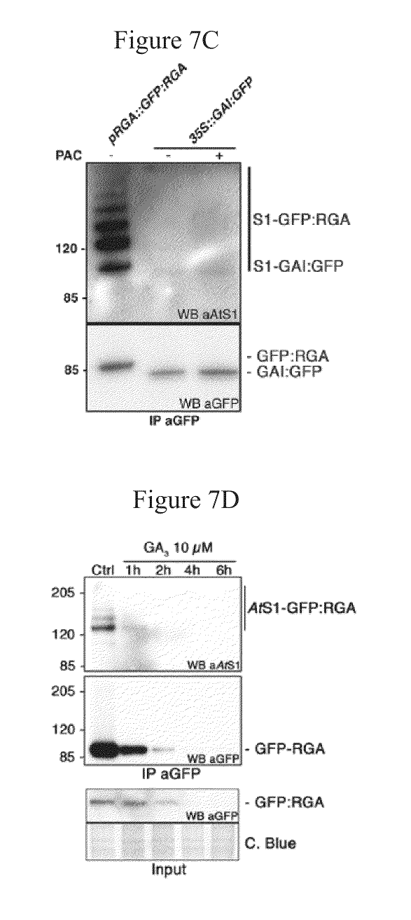

Applicants next addressed whether SUMOylation of DELLA proteins could provide such a GA independent mechanism for stabilising DELLAs. Taking advantage of a well-established transgenic line in which RGA is expressed as a functional GFP fusion under the endogenous RGA promoter (12) (pRGA::GFP:RGA) Applicants immunopurified GFP:RGA protein under stringent conditions using GFP antibody-coated beads. GFP antibody detection revealed several forms of GFP:RGA in the immunoprecipitate migrating at higher antibodies indicated that these higher molecular weight forms of GFP:RGA were conjugated to SUMO1 (FIG. 2a). To confirm that these SUMOylated GFP:RGA forms were targets for OTS1 SUMO protease action Applicants incubated the immunoprecipitate with purified OTS1 SUMO protease as well as a catalytically inactive form of OTS1 (OTS1C526S). This treatment resulted in the dramatic reduction of the higher molecular weight, anti-SUMO1 cross-reacting bands only in the tubes containing wild-type OTS1, strongly indicating that OTS1 SUMO protease directly deSUMOylates DELLA proteins (FIG. 2b). Further controls excluded the possibility that the SUMOylated forms of GFP:RGA could be derived from non-specific SUMOylation of GFP (FIG. 7a, b).

If SUMOylation represented an important regulatory mechanism for DELLA stability in plants, Applicants would expect the site of conjugation to be highly conserved in DELLA sequences across all plant species. Using a bacterial SUMOylation system (34) Applicants established that lysine 65 is the critical amino acid for SUMO attachment on RGA (FIG. 2c). Strikingly, this SUMOylation site lysine residue is conserved across all DELLA proteins in Arabidopsis and other plant species including monocots (FIG. 2d). Notably however, the N-terminal residue immediately adjacent to the highly conserved K residue varies between RGA and GAI. IN RGA, this is L, but in GAI and GAI orthologues in crops, as well as in RGL2 and 3, this is Q. Applicants believe that it is this difference which mediates a different effect of expression of 35S:RGA(k/r):GFP and 35S:GAI(k/r):GFP respectively. Applicants postulate that disruption of the SUMOylation site in RGA increases stability of the SUMOylation site whereas manipulation of the K in GAI makes it more unstable and prevents SUMOylation. In any case, Applicants also demonstrated that the other major growth regulating DELLA protein, GAI is also SUMOylated in vivo (FIG. 7c). This remarkable conservation of the SUMO site in DELLAs from divergent plant species is consistent with this mechanism playing a critical role in DELLA signaling. To gain more insight into the role of DELLA SUMOylation and its interplay with the non-SUMOylated DELLA, Applicants analysed the pattern of accumulation of the SUMOylated RGA pool in conditions known to stimulate DELLA accumulation. Applicants found that conditions that promote DELLA accumulation (high salinity) also enhanced SUMOylated DELLA abundance (FIG. 2e). However GA treatment induced a rapid disappearance of both SUMOylated and non-SUMOylated RGA forms indicating that SUMOylation of DELLAs acts primarily to increase DELLA abundance (FIG. 7d). Applicants next sought to establish the mechanistic role of SUMOylation on DELLA protein accumulation. Applicants previously showed that RGA protein levels are increased in ots1 ots2 compared to wild type. Applicants further confirmed this was also the case for GFP:RGA fusion proteins by crossing the pRGA::GFP:RGA plant lines with ots1 ots2 mutants. This allowed us to compare GFP:RGA and SUMOylated GFP:RGA protein levels in the presence and absence of OTS1 and OTS2 activities. Applicants observed as expected more SUMOylated GFP:RGA in ots1 ots2 mutants compared to wild-type and this was associated with higher GFP:RGA levels (FIG. 2f). This effect on SUMOylated and non-SUMOylated GFP:RGA was enhanced when ots1 ots2 plants were grown in the presence of salt (FIG. 2g). Applicants' data indicate that stress-related OTS SUMO proteases are major regulators of DELLA levels in vivo.

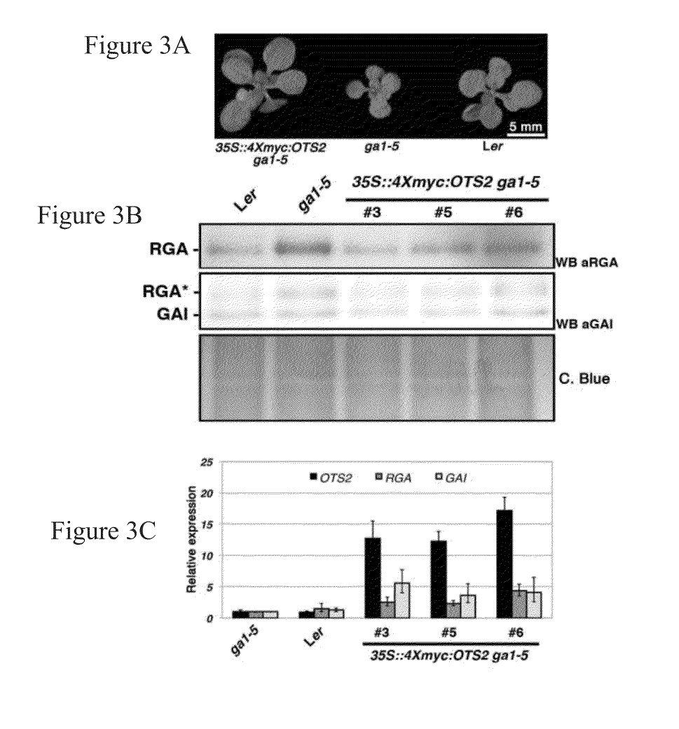

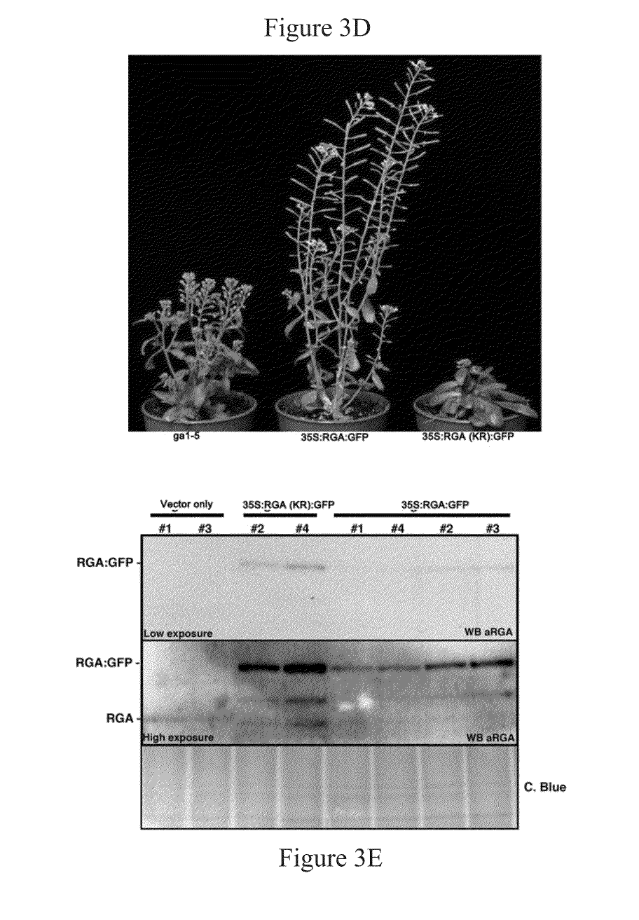

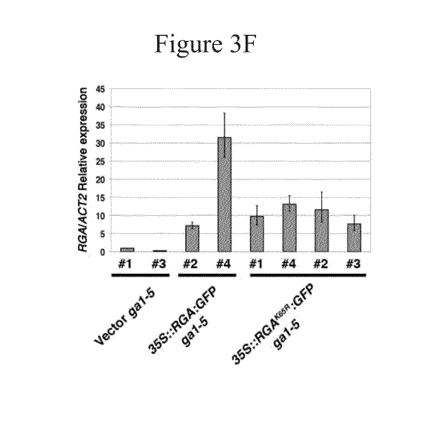

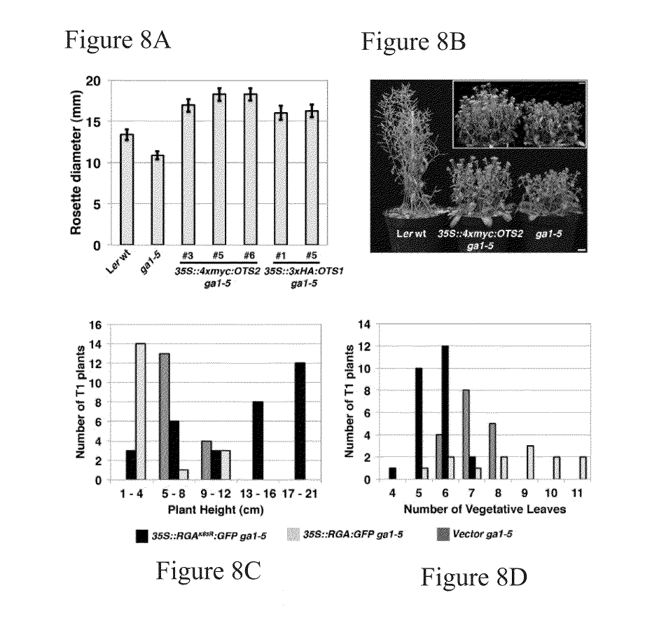

To elucidate the mechanism for how SUMOylation affects the accumulation of DELLAs in a GA independent manner, Applicants first produced transgenic plants that over-expressed OTS1 and OTS2 in the gal-5 background (which is partially deficient in bioactive GA and therefore allowing accumulation of DELLAs). Over-expression of OTS1 or OTS2 SUMO proteases in the gal-5 genetic background attenuated the growth repression mediated by higher DELLA protein levels in these GA-deficient plants (FIG. 3a, FIG. 8a, b). Western blot analysis showed a clear decrease in DELLA protein accumulation indicating that continuous deSUMOylation by OTS results in lower DELLA levels (FIG. 3b). Conversely DELLA transcript levels were up-regulated in OTS2 overexpressing lines, as a result of an established negative feedback loop initiated by lowering DELLA protein levels (35) (FIG. 3c). As gal-5 plants produce very low levels of bioactive GAs it is unlikely that an increase in GA levels can account for this derepression of growth (FIG. 3a). Hence, these data provide further evidence for the existence of an alternative mechanism working via SUMOylation that directly modifies DELLA levels.

To test this new DELLA regulatory mechanism further, Applicants produced transgenic plants ectopically expressing either a wild-type copy of RGA fused to GFP (35S::RGA:GFP) or mutagenized versions of RGA lacking the relevant SUMO attachment site lysine (35S::RGAK65R:GFP) in the gal-5 genetic background. As expected, overexpression of RGA resulted in plants with a phenotype that is very similar to the wild type. This is expected as it has been shown that overexpression of RGA does not cause dwarfism, but over expression of GAI does. RGA was originally identified because loss-of-function mutations cause partial suppression of the dwarf phenotype conferred by the GA deficiency mutation, gal-3. Whilst absence of RGA (in a rga-24 gal-3 double mutant) causes a gal-3 mutant to grow taller than it does in the presence of RGA, the absence of GAI (in a gai-t6 gal-3 double mutant) does not have such an obvious effect on stem elongation of gal-3. This suggests that RGA plays a predominant role in the repression of stem elongation. However, overexpression of RGA (in transgenic 35S:RGA lines) does not confer an obvious phenotype on WT Arabidopsis plants. Thus, overexpression of GAI results in a different phenotype compared to overexpression of RGA (FIG. 3d, FIG. 8c, d). In contrast, plants expressing RGAK65R were dwarf compared to those expressing RGA, but also compared to vector control plants. For GAI overexpressing plants, as expected, the plants show a dwarf phenotype. Plants overexpressing GAIK65R:GFP were similar to the wild type.

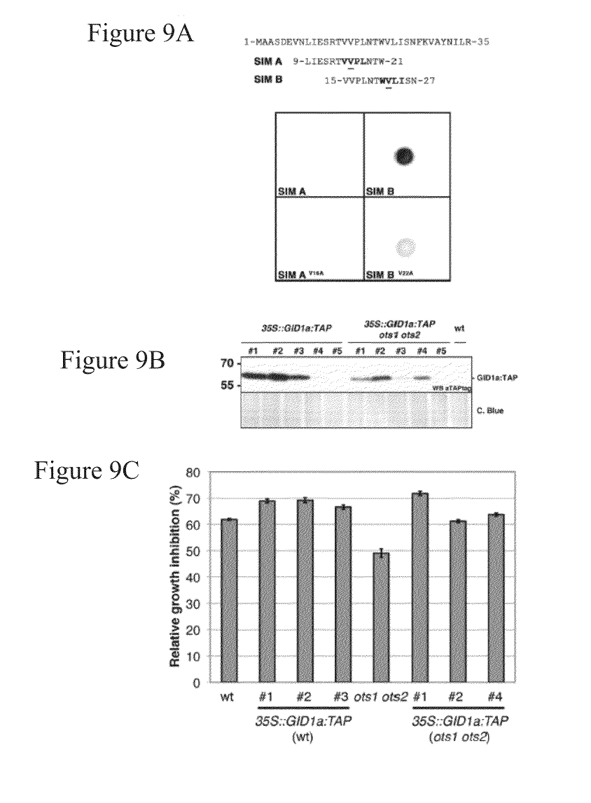

Applicants next investigated whether the SUMOylated DELLA could interfere with the function of other components of the GA signaling pathway, namely GID16 and SLEEPY117. Closer inspection of the GID1 protein sequence revealed a functional SUMO interaction motif (SIM) at its N-terminus (FIG. 4a, FIG. 9a). Applicants directly demonstrated that recombinant GST-tagged GID1a can bind to SUMO1 from Arabidopsis in a GA-independent manner (FIG. 4b). Applicants then tested whether SUMOylated DELLA had similar binding properties to GID1a as did uncoupled SUMO1. Recombinant GST-tagged GID1a was incubated with plant-derived DELLA mixture (consisting of both SUMOylated and non-SUMOylated forms). Applicants found that SUMOylated RGA could bind to GST:GID1a even in the absence of GA indicating that the SUMO1 protein that is bound to DELLAs mediates this GA independent interaction with GID1a (FIG. 4c).

This result allowed us to postulate that a relatively small pool of SUMOylated DELLA could stabilize the larger pool of unmodified DELLA by titrating GID1a protein. To test the hypothesis that GID1a protein is rate limiting for this process Applicants overexpressed GID1a in ots1 ots2 double mutant plants where there are higher levels of both SUMOylated DELLA and non-SUMOylated DELLAs. Applicants anticipated that by overexpressing GID1a Applicants should overcome the sensitivity to salt and the GA-biosynthesis inhibitor paclobutrazol (PAC) mediated by increased DELLA levels in the ots1 ots2 double mutant. The dramatic delay in germination in ots1 ots2 mutants during PAC treatment is suppressed when GID1a is overexpressed in this genetic background (FIG. 4d). Similarly the greater inhibition of root growth in ots1 ots2 double mutants on high salinity can be ameliorated by enhancing the expression of GID1a (FIG. 4e, FIG. 9b, c). Applicants' data indicates that GID1 is rate limiting in maintaining the steady state levels of DELLA proteins. SUMOylation of DELLAs then acts as a ‘decoy’ to enhance the levels of non-SUMOylated DELLAs by sequestering the GA receptor GID1 (FIG. 4f). The discovery of an alternative mechanism regulating DELLA abundance reported in this study provides an important new insight into the central role of DELLAs in controlling plant growth.

### Methods Summary

All plants used in this study were in either the Col-0 or Landsberg erecta backgrounds and multiple mutants were generated by crossing. Transgenic plants were obtained by transformation of the relevant genetic background by floral dip. T-DNA lines seeds were obtained from the Nottingham Arabidopsis stock centre. The procedures for Arabidopsis plant growth, protein blots and recombinant protein production in E. coli were previously described (32). GA measurements were done as previously illustrated5. For the germination assay, GA3 and PAC were supplemented to the plant growth medium. Seeds were stratified on plates for three days before exposure to light and scored after 3 to 5 days. For proteins and transcripts analysis, surface sterilised seeds were stratified and germinated on filter papers laid on plant agar growth medium and pooled seedlings (20-40) were harvested after 8 to 10 days. Full details of the constructs and plant genotypes used in this study are available in the full methods section. Primers used in this study are listed in Table 1.

### Plant Material

The ots1-1 ots2-1 double mutants plants were previously described (36). The ots2-2 mutant is a novel T-DNA insertion allele (SALK_{—}067439) resulting in no detectable full length OTS2 transcript. The ots2-2 allele was detected by PCR on genomic DNA using primers LC15 and LC18, flanking the T-DNA insertion region and LBa1 (SALK T-DNA primer) in combination with LC15, which were insertion-specific. The null rga mutant allele used in this study (dubbed rga-100) derives from a T-DNA insertion (SALK_{—}089146C). Homozygous plants were genotyped with primers LC69 and LC70, flanking the T-DNA insertion region and LBa1 (SALK T-DNA primer) and LC70, which were insertion allele specific. The null gai mutant allele used in this study (dubbed gai-100) derived from a TDNA insertion (SAIL_{—}587_C02). Homozygous plants were resistant to the herbicide Basta and confirmed by PCR using with primers LC80 and LC81, flanking the T-DNA insertion region and LB1 (SAIL T-DNA primer) and LC81, which were insertion allele specific. The gal-5 mutants were obtained from NASC and the pRGA::GFP:RGA line (Ler background) (37), 35S::NPR1:GFP npr1 (38) plants were previously described.

### Plasmid Construction

The 35S::3XHA:OTS1 and 35S::4Xmyc:OTS2 constructs were generated by recombining the plasmids pLCG1 and pLCG14 (harbouring the OTS1 and OTS2 cDNAs, respectively) with the binary GATEWAY destination vectors pGWB15 and pGWB18 (respectively) (39) via LR Recombinase II (Invitrogen). The RGA ORF (and part of the 5′ UTR region) was amplified by PCR from whole cDNAs from seedlings with oligos LC75 and LC76 and cloned into pENTR/D-TOPO (Invitrogen) to yield pLCG67. The rgaK65R allele was generated by amplifying pLCG67 with mutagenic oligos LC77 and LC78 (which carried a single base pair change) according to the QuikChange Site-Directed Mutagenesis Kit Directions (Stratagene) and the resulting plasmid (pLCG68) was sequenced. The GAI ORF was amplified by PCR from whole cDNAs from seedlings with oligos LC80 and LC81 and cloned into pENTR/D-TOPO (Invitrogen) to yield pLCG69. The 35S::RGA:GFP, 35S::GAI:GFP, 35S::GAIK65R:GFP and 35S::RGAK65R:GFP constructs were generated by recombining the plasmids pLCG67, pLCG68 and pLCG69 with the binary GATEWAY destination vectors pGBPGWG (40) via LR Recombinase II (Invitrogen). The GID1a ORF was amplified by PCR from whole cDNAs from seedlings with oligos LC73 and LC74 and cloned into pENTR/D-TOPO (Invitrogen) to yield pLCG66.

Plants expressing 35S::GAIK65R:GFP are tested under stress conditions, including high salinity and water deficit (drought). The high salinity test is carried out by growing seedlings on MS agar plates for 14 days in 100 mM NaCl. The drought test is carried out on soil grown plants. Plants are grown with normal watering for 2 weeks after which water is withdrawn for 3 weeks. Plants are analysed for survival and biomass production. Furthermore, plants (including controls) are watered once with a known quantity of water e.g. (50 ml.) and recovery of plant growth and productivity (biomass production seed yield etc.) is monitored. The 35S::GID1a:TAP construct were generated by recombining the plasmids pLCG66 with the binary GATEWAY destination vectors pEarleyGate 205 (41) via LR Recombinase II (Invitrogen). The fusion GST:GID1a construct was generated by recombining the plasmids pLCG66 with the GATEWAY destination vectors pDEST15 via LR Recombinase II (Invitrogen). 35S::GID1V22A constructs were generated in destination vector pEarly vector 201 (with a N-terminal HA tag and expressed in wild type plants and the ots1:ots2 background respectively. Seedlings were grown on plates using 75 mM NaCl for 14 days.

### Protein Extraction, Immunoprecipitation and Antibodies

Total proteins were extracted by homogenizing fresh Arabidopsis seedlings in the presence of ice cold extraction buffer—150 mM NaCl, 1% Igepal CA-630, 0.5% sodium deoxycholate, 0.1% SDS, 1 mM EDTA, 50 mM Tris HCl, pH 8.0 and freshly added protease inhibitor cocktail (Roche) and 10 mM N-ethylmaleimide (NEM). The homogenates were clarified by spinning 10 min at 4° C. at 13000×g and the supernatant quantified with the Bradford assay. Approximately 2-3 mg were subjected to immunoprecipitation using the μMACS GFP Isolation Kit (Miltenyi biotech) according to the manufacturers' instructions. Magnetic beads were washed four times with extraction buffer and once with 20 mM Tris HCl, pH 7.5 before elution with hot SDSPAGE buffer (50 mM Tris HCl, pH 6.8, 50 mM DTT, 1% SDS, 1 mM EDTA, 0.005% bromphenol blue, 10% glycerol). For combined RNA and protein analysis, the protein fraction was obtained by following the TRIzol (life technologies) reagent protocol. The isopropanol precipitated protein pellet was washed three times in 0.3 M Guanidine hydrochloride, 95% ethanol before solubilisation in 6 M Urea, 0.1% SDS. Total proteins were quantified with the Bradford reagent and an equal amount of proteins was precipitated with five volumes of cold acetone. The pellet was then resuspended in SDSPAGE loading buffer (containing Urea 4 M) before loading. To reveal the SUMOylation pattern at high resolution, the immunoprecipitates were resolved on precast 4-8% Tris-Acetate NuPAGE gels (Invitrogen) otherwise proteins (50-100 μg) were resolved on standard 8% SDS-PAGE gels. Proteins were blotted and probed with AtSUMO1 and TAPtag antibodies as previously described. The RGA and GAI antibodies were made in sheep and used at a 1:2000 dilution. The rabbit GFP ad GST antibodies were bought from abcam and used at a 1:4000 dilution.

### GST Pull Down Assay

For recombinant proteins, affinity purified GST:GID1a (0.1 μg) or GST were mixed with His:AtSUMO1 (0.1 μg) and incubated in 1× reaction buffer (Gamborg's B5—minimal organics, 50 mM NaCl, 0.05% Igepal CA-630, 1 mM DTT, 50 mM Tris HCl, pH 7.5). GA3 was added at a final concentration of 10 μM. Proteins were pulled-down using the μMACS GST Isolation Kit, according to the manufacturers' instruction (Miltenyi biotech). Plant GFP:RGA proteins were affinity captured as previously described and eluted from anti-GFP magnetic beads with 0.1% Triethanolamine, 0.1% Triton X100 and neutralised with 100 mM MES (pH 2.5). The eluate was dialyzed against 50 mM Tris HCl, pH 7.5, 50 mM NaCl, 1 mM DTT. Plant purified GFP:RGA proteins were split into different tubes and incubated with recombinant GST:GID1a (0.1 μg) or GST proteins in 1× reaction buffer (with freshly added protease inhibitor cocktail) in the presence or absence of 10 μM GA3. GST-bound proteins were pulled-down using the μMACS GST Isolation Kit, washed four times with 1× reaction buffer and eluted according to the manufacturers' instruction.

### Far-Western Assay

Peptides corresponding to the putative SIMs in GID1 were purchased from Cambridge Research Biochemicals. 1 μg of each peptide was spotted on a PVDF membrane. Membranes were washed in 100% Ethanol, equilibrated in TBST (25 mM Tris HCl, pH 8.0, 150 mM NaCl, 0.05% Tween 20) and blocked in TBST-Milk 5%. Peptides were probed overnight at 4° C. with recombinant His:proAtSUMO1 (10 μg/ml), washed and subsequently probed with SUMO1 antibodies for standard chemoluminescence-based detection.

On-Column deSUMOylation Assay

GFP:RGA proteins were affinity captured from total proteins extracts of pRGA::GFP:RGA transgenic plants with the μMACS GFP Isolation Kit. Magnetic beads were eluted from the columns with 50 μl of 20 mM Tris HCl, pH 7.5 and split into different tubes. Purified GFP:RGA proteins were incubated with 5-10 μg of recombinant OTS1 or OTS1C526S, or 300 ng of GST tagged human SENP1 or SENP (42) (catalytic domain) (Enzo life sciences). After incubation (typically 1-2 hours at room temperature), the beads were applied to the column, washed and bound proteins eluted with SDSPAGE loading buffer.

### Transcript Analysis

Plant material (young seedlings) was pulverized with a pestle in the presence of liquid nitrogen and total RNA was extracted with the TRIzol reagent (life technologies). First strand cDNA synthesis was carried out from 500 ng of total RNA using the VILO reverse transcriptase kit (Invitrogen). cDNA was diluted 5 times, mixed with the FAST Sybr Green master mix (Applied Biosystem) and used for qPCR with a 7900HT Fast Realtime PCR (Applied Biosystem). To detect RGA transcript levels, oligonucleotides lcm26 and lcm27 were used; for GAI, oligonucleotides lcm28 and lcm29. OTS2 transcript levels were analysed using oligonucleotides LC85 and LC86. Oligonucleotides mr37 and mr38 amplifying ACT2 (At3g18780) were used for normalization.

### Bioinformatics

The SUMO site in DELLAs was identified by using a combination of in vitro SUMOylation system (Okada et al., Plant Cell Physiol 50, 1049-1061), Mass spectrometry and bioinformatics based on homology to related DELLAs in other plant species.

### Analysis of Jaz6 Protein

The SUMOylation site in JAZ6 was identified and mutated. A Western blot of SUMOylation screen of JAZ6, with three K to R mutants was carried out. Blot shows that JAZ6 is SUMOylated and that mutating lysine 221 to arginine (K221R) abolishes SUMOylation, therefore lysine 221 is likely the site of SUMOylation. JAZ6 fused to maltose binding protein (MBP) and probed with anti MBP.

### Analysis of Phy-B Mutant

A SUMOylation screen of phytochrome B (PHYB-GFP), with two mutant forms, PHY-B (S86D), which is the hyperphosphorylated form of PHYB, and PHY-B S86A, the non-phosphorylated form was carried out by Western Blot. The blot shows that PHY-B is hyperSUMOylated during middle of day then end of night. The hyperphosphorylated mutant form cannot be SUMOylated even in the middle of day time point indicating interdependence of phosphorylation and SUMOylation mechanisms.

### ARF19 and ARF17 Analysis

### In Vitro SUMO Assays.

The SUMO cascade has been reconstituted into E. coli by Okada et al. (2009) and allows a recombinant protein of choice (in this case ARF7 and 19) to be co-expressed and tested for SUMOylation, either by a molecular weight increase in the protein under investigation or by probing with anti-SUMO antibodies. Their system consists of three co-expressed plasmids. The first two contain genes for the SUMO cascade enzymes and the third is used to express the gene to be tested. SUMO, the E1 dimer and E2 but not any E3 are expressed by the system. E3 is not essential for SUMOyation in this assay, especially as the SUMO cascade enzymes are expressed at very high concentrations and rate limitations of the reaction are overcome. All proteins expressed in this system are only inducible after addition of IPTG. The defective form of SUMO (SUM-AA) with the diglycine C-terminus mutated to dialanine that cannot be ligated to a target is included as a negative control.

To confirm that ARFs were indeed SUMOylated in vitro, the ARF19 and 7 cDNAs were cloned as GST fusions for expression into the reconstituted SUMOylation system in E. coli. Once the proteins were induced by IPTG for 2 hours in the SUMO system the E. Coli lysates were prepared by centrifugation. The E. Coli cells were lysed using lysozyme and sonication to prepare total protein extracts. These extracts were subjected to immunoprecipitation with anti-GST antibodies to immunopurify GST-ARF7 or GST-ARF19. The immunoprecipitates were subjected to electrophoresis and the proteins were blotted onto PVDF membranes. The membranes were than probed with anti-SUMO1 antibodies to detect SUMOylation of GST-ARF7 or 19.

FIG. 16 shows a western blot probed with anti-SUMO1 antibodies (as detailed below). The negative controls (−, AA SUMO mutants) show no conjugation of SUMO to ARF19 or 7. The + lanes contain wildtype SUMO and they show a characteristic “ladder’ of SUMO conjugation ARF19 however this is not so clear with ARF7. This maybe due to poor immunoprecipitation of ARF7 or ARF7 is a poor substrate for SUMOylation.

Western Blotting to Detect ARF19 Protein from Arabidopsis Total Protein Extracts

Arabidopsis seedlings were frozen in liquid nitrogen and homogenized in E buffer (125 mM Tris-HCl, pH 8.8, 1% [w/v] SDS, 10% [v/v] glycerol, and 50 mM sodium metabisulfite) (Martinez-Garcia et al., 1999) with freshly added 5 mM NEM—N-Ethylmaleimide and protease inhibitor cocktail (Roche mini-PI tablets) (1 tablet in 20 mls of Extraction Buffer). The homogenate was microcentrifuged at 16,000 g for 5 min at 4 degrees Celsius and the supernatant was quantified with Bradford reagent before mixing with 4× SDS-PAGE loading buffer. Equal amounts of proteins for each sample were loaded onto a 4 to 12% NuPAGE Novex Bis-Tris gel run in MES-SDS buffer (Invitrogen) or a standard SDS-PAGE gel. Proteins were then transferred to a polyvinyl difluoride membrane (Bio-Rad) for immunoblot analysis.

### Probing Membranes

Filters were blocked in TTBS-milk (5% [w/v] dry nonfat milk, 10 mM Tris-HCl, pH 8, 150 mM NaCl, and 0.1% [v/v] Tween 20) before incubation with primary antibody anti-sheep ARF19 or anti-SUMO1 (for in vitro SUMO assays). Filters were washed in TTBS and incubated with secondary antibody (anti-rabbit horseradish peroxidase conjugate [Sigma-Aldrich]) or anti-Sheep horseradish peroxidase conjugate diluted 1:20,000 in TTBS-milk. Filters were washed and incubated with the horseradish peroxidase substrate (Immobilon Western; Millipore) before exposure to film (Kodak).

### Barley Transformation

The constructs for barley transformation contain GAI (wildtype) and mutant GAI (K65R, SUMO site mutant) and are expressed under the control of the ubiquitin promoter in barley. The vector is pBRACT214 with kanamycin resistance in bacteria and hygromycin in plants. Salt stress experiments in 10 day old seedling are carried out in pots to ascertain that the barley transgenics show improved salt tolerance. For general phenotypic analysis, plants are grown under glasshouse conditions and GAI and GAI (K65R)-ox barley lines (10 plants per independent transgenic line) are monitored for changes in growth rate, plant height, heading time, number of tillers, spike phenotype, grain phenotype and yield. Untransformed plants and plants with no transgene expression (null segregants) as well as vector only transformed plants are used as controls. Biomass is assayed. Agrobacterium strain AGL1 containing pBract vectors is used. pBract vectors are based on pGreen and therefore need to be co-transformed into Agrobacterium with the helper plasmid pSoup. To enable the small size of pGreen, the pSa origin of replication required for replication in Agrobacterium, is separated into its' two distinct functions. The replication origin (ori) is present on pGreen, and the trans-acting replicase gene (RepA) is present on pSoup. Both vectors are required in Agrobacterium for pGreen to replicate. pBract vector DNA and pSoup DNA were concurrently transferred to AGL1 via electroporation. A standard Agrobacterium inoculum for transformation is prepared. A 400 μl aliquot of standard inoculum is removed from −80° C. storage, added to 10 ml of MG/L medium without antibiotics and incubated on a shaker at 180 rpm at 28° C. overnight. This full strength culture is used to inoculate the prepared immature embryos. A small drop of Agrobacterium suspension is added to each of the immature embryos on a plate. The plate is then tilted to allow any excess Agrobacterium suspension to run off. Immature embryos is then gently dragged across the surface of the medium (to remove excess Agrobacterium) before being transferred to a fresh CI plate, scutellum side down. Embryos are co-cultivated for 3 days at 23-24° C. in the dark.

Donor plants of the spring barley, Golden Promise, are grown under controlled environment conditions with 15° C. day and 12° C. night temperatures as previously described (43). Humidity is about 80% and light levels about 500 μmol.m⁻².s⁻¹ at the mature plant canopy level provided by metal halide lamps (HQI) supplemented with tungsten bulbs. Immature barley spikes are collected when the immature embryos were 1.5-2 mm in diameter. Immature seeds are removed from the spikes and sterilised as previously described (44). The immature embryos are exposed using fine forceps and the embryonic axis removed. The embryos are then plated scutellum side up on CI medium containing 4.3 g l⁻¹ Murashige & Skoog plant salt base (Duchefa), 30 g l⁻¹ Maltose, 1.0 g l⁻¹ Casein hydrolysate, 350 mg l⁻¹ Myo-inositol, 690 mg l⁻¹ Proline, 1.0 mg l⁻¹ Thiamine HCl, 2.5 mg l⁻¹ Dicamba (Sigma-Aldrich) and 3.5 g l⁻¹ Phytagel, with 25 embryos in each 9 cm Petri dish. After co-cultivation, embryos are transferred to fresh CI plates containing 50 mg l⁻¹ hygromycin, 160 mg l⁻¹ Timentin (Duchefa) and 1.25 mg l⁻¹ CuSO₄.5H₂O. Embryos are sub-cultured onto fresh selection plates every 2 weeks and kept in the dark at 24° C. After 4-6 weeks, embryos are transferred to transition medium (T) containing 2.7 g l⁻¹ Murashige & Skoog modified plant salt base (without NH₄NO₃) (Duchefa), 20 g l⁻¹ Maltose, 165 mg l⁻¹ NH₄NO₃, 750 mg l⁻¹ Glutamine, 100 mg l⁻¹ Myo-inositol, 0.4 mg l⁻¹ Thiamine HCl, 1.25 mg l⁻¹ CuSO₄.5H₂O, 2.5 mg l⁻¹ 2,4-Dichlorophenoxy acetic acid (2,4-D) (Duchefa), 0.1 mg l⁻¹ 6-Benzylaminopurine (BAP), 3.5 g l⁻¹ Phytagel, 50 mg l⁻¹ Hygromycin and 160 mg l⁻¹ Timentin in low light. After a further 2 weeks, embryo derived callus are transferred to regeneration medium in full light at 24° C., keeping all callus from a single embryo together. Regeneration medium is the same as the transition medium but without additional copper, 2,4-D or BAP. Once regenerated plants shoots of about 2-3 cm in length are transferred to glass culture tubes containing CI medium, without dicamba or any other growth regulators but still containing 50 mg l⁻¹ hygromycin and 160 mg l⁻¹ Timentin.

### Salt Stress

Two-week-old control and Ti generation Hv GAI and GAI K65R-ox plants are initially subjected to 10 days of salt stress by watering with 100 mM NaCl in pots. During this period Applicants determine the onset of salt stress symptoms such as loss of turgor, leaf rolling and loss of chlorophyll and compare them to control plants. Plants are assessed for recovery after 1 and 3 weeks of re-watering with no salt, and stress-tolerant plants will be transferred to the glasshouse for generation of seeds to determine yield.

### REFERENCES

1. Achard, P. et al. Integration of plant responses to environmentally activated phytohormonal signals. Science 311, 91-94 (2006).
2. Achard, P. et al. DELLAs contribute to plant photomorphogenesis. Plant Physiol. 143, 1163-1172 (2007).
3. Navarro, L. et al. DELLAs control plant immune responses by modulating the balance of jasmonic acid and salicylic acid signaling. Curr. Biol. 18, 650-655 (2008).
4. Hou, X., Lee, L. Y. C., Xia, K., Yan, Y. & Yu, H. DELLAs modulate jasmonate signaling via competitive binding to JAZs. Dev Cell 19, 884-894 (2010).
5. Griffiths, J. et al. Genetic characterization and functional analysis of the GID1 gibberellin receptors in Arabidopsis. Plant Cell 18, 3399-3414 (2006).
6. Ueguchi-Tanaka, M. et al. GIBBERELLIN INSENSITIVE DWARF1 encodes a soluble receptor for gibberellin. Nature 437, 693-698 (2005).
7. Ueguchi-Tanaka, M. et al. Molecular interactions of a soluble gibberellin receptor, GID1, with a rice DELLA protein, SLR1, and gibberellin. Plant Cell 19, 2140-2155 (2007).
8. Murase, K., Yoshinori Hirano, Sun, T.-P. & Toshio Hakoshima Gibberellin induced DELLA recognition by the gibberellin receptor GID1. Nature 456, 459 (2008).
9. Asako Shimada et al. Structural basis for gibberellin recognition by its receptor GID1. Nature 456, 520 (2008).
10. Willige, B. C. et al. The DELLA domain of GA INSENSITIVE mediates the interaction with the GA INSENSITIVE DWARF IA gibberellin receptor of Arabidopsis. Plant Cell 19, 1209-1220 (2007). “DELLA” disclosed as SEQ ID NO: 70.
11. Dill, A. & T Sun Synergistic derepression of gibberellin signaling by removing RGA and GAI function in Arabidopsis thaliana. Genetics 159, 777-785 (2001).
12. Silverstone, A. L. et al. Repressing a repressor: gibberellin-induced rapid reduction of the RGA protein in Arabidopsis. Plant Cell 13, 1555-1566 (2001).
13. Fu, X. et al. The Arabidopsis mutant sleepy1gar2-1 protein promotes plant growth by increasing the affinity of the SCFSLY1 E3 ubiquitin ligase for DELLA protein substrates. Plant Cell 16, 1406-1418 (2004).
14. Itoh, H., Ueguchi-Tanaka, M., Sato, Y., Ashikari, M. & Matsuoka, M. The Gibberellin Signaling Pathway Is Regulated by the Appearance and Disappearance of SLENDER RICE1 in Nuclei. Plant Cell 14, 57 (2002).
15. Wang, F. et al. Biochemical insights on degradation of Arabidopsis DELLA proteins gained from a cell-free assay system. Plant Cell 21, 2378-2390 (2009).
16. Fu, X. et al. Gibberellin-mediated proteasome-dependent degradation of the barley DELLA protein SLN1 repressor. Plant Cell 14, 3191-3200 (2002).
17. McGinnis, K. M. et al. The Arabidopsis SLEEPY1 gene encodes a putative F-box subunit of an SCF E3 ubiquitin ligase. Plant Cell 15, 1120-1130 (2003).
18. Achard, P., Renou, J.-P., Berthome, R., Harberd, N. P. & Genschik, P. Plant DELLAs restrain growth and promote survival of adversity by reducing the levels of reactive oxygen species. Curr. Biol. 18, 656-660 (2008).
19. Peng, J. et al. The Arabidopsis GAI gene defines a signaling pathway that negatively regulates gibberellin responses. Genes Dev. 11, 3194-3205 (1997).
20. Peng, J. et al. Green revolution genes encode mutant gibberellin response modulators. Nature 400, 256 (1999).
21. Silverstone, A. L., Mak, P. Y., Martinez, E. C. & Sun, T. P. The new RGA locus encodes a negative regulator of gibberellin response in Arabidopsis thaliana. Genetics 146, 1087-1099 (1997).
22. Ikeda, A. et al. slender rice, a constitutive gibberellin response mutant, is caused by a null mutation of the SLR1 gene, an ortholog of the height-regulating gene GAI/RGA/RHT/D8. Plant Cell 13, 999-1010 (2001).
23. de Lucas, M. et al. A molecular framework for light and gibberellin control of cell elongation. Nature 451, 480-484 (2008).
24. Feng, S. et al. Coordinated regulation of Arabidopsis thaliana development by light and gibberellins. Nature 451, 475-479 (2008).
25. Elrouby and Coupland. Proteome-wide screens for small ubiquitin-like modifier (SUMO). PNAS, 107 (40) 17415-17420
26. Liu et al Neuroscience. 2002; 115(3):829-37).
27. Fleet and Sun, Current Opinion in Plant Biology 2005, 8:77-85
28. Bolle C (2004) The role of GRAS proteins in plant signal transduction and development. Planta 218:683-692).
29. Miura, K. et al. Sumoylation of ABI5 by the Arabidopsis SUMO E3 ligase SIZ1 negatively regulates abscisic acid signaling. Proc Natl Acad Sci USA 106, 5418-5423 (2009).
30. Lee, M. H. et al. SUMO-specific protease SUSP4 positively regulates p53 by promoting Mdm2 self-ubiquitination. Nat. Cell Biol. 8, 1424-1431 (2006).
31. Conti, L. et al. Small ubiquitin-like modifier proteases OVERLY TOLERANT TO SALT1 and -2 regulate salt stress responses in Arabidopsis. Plant Cell 20, 2894-2908 (2008).
32. Lee, S. et al. Gibberellin regulates Arabidopsis seed germination via RGL2, a GAI/RGA-like gene whose expression is up-regulated following imbibition. Genes Dev. 16, 646-658 (2002).
33. Tyler, L. et al. Della proteins and gibberellin-regulated seed germination and floral development in Arabidopsis. Plant Physiol. 135, 1008-1019 (2004).
34. Okada, S. et al. Reconstitution of Arabidopsis thaliana SUMO pathways in E. coli:functional evaluation of SUMO machinery proteins and mapping of SUMOylation sites by mass spectrometry. Plant Cell Physiol 50, 1049-1061 (2009).
35. Ariizumi, T., Murase, K., Sun, T.-P. & Steber, C. M. Proteolysis-independent downregulation of DELLA repression in Arabidopsis by the gibberellin receptor GIBBERELLIN INSENSITIVE DWARF1. Plant Cell 20, 2447-2459 (2008).
36. Conti, L. et al. Small ubiquitin-like modifier proteases OVERLY TOLERANT TO SALT1 and -2 regulate salt stress responses in Arabidopsis. Plant Cell 20, 2894-2908 (2008).
37. Silverstone, A. L. et al. Repressing a repressor: gibberellin-induced rapid reduction of the RGA protein in Arabidopsis. Plant Cell 13, 1555-1566 (2001).
38. Kinkema, M., Fan, W. & Dong, X. Nuclear localization of NPR1 is required for activation of PR gene expression. Plant Cell 12, 2339-2350 (2000).
39. Nakagawa, T. et al. Development of series of gateway binary vectors, pGWBs, for realizing efficient construction of fusion genes for plant transformation. J. Biosci. Bioeng. 104, 34-41 (2007).
40. Zhong, S. et al. Improved plant transformation vectors for fluorescent protein tagging. Transgenic Research 17, 985 (2008).
41. Earley, K. W. et al. Gateway-compatible vectors for plant functional genomics and proteomics. Plant J 45, 616-629 (2006).
42. Bailey, D. & O'Hare, P. Characterization of the localization and proteolytic activity of the SUMO-specific protease, SENP1. J. Biol. Chem. 279, 692-703 (2004).
43. Harwood W A, Ross S M, Cilento P, Snape J W: The effect of DNA/gold particle preparation technique, and particle bombardment device, on the transformation of barley (Hordeum vulgare). Euphytica 2000, 111:67-76
44. Okushima et al: ARF7 and ARF 19 regulate lateral root formation via direct activation of LBD/ASL genes in Arabidopsis, The Plant Cell, 19, 118-130 (2007)
45. Skirycz et at Survival and growth of Arabidopsis plants given limited water are not equal volume 29 number 3 March 2011, Nature biotechnology
46. Greaves et al Improving suboptimal temperature tolerance in maize—the search for variation Journal of Experimental Botany, Vol. 47, No. 296, pp. 307-323, March 1996

TABLE 1
List of primers used in this study
Oligo Sequence (5′-3′) Amplicon

| | |
|---|---|
| LC15 | TTAATCTGTTTGGTTACCCTTGCGG OTS2 SEQ ID No. 21 |
| LC18 | GACAGGGATGCATATTTTGTGAAG OTS2 SEQ ID No. 22 |
| LC69 | CCGTCGGAGCTTTATTCTTG RGA SEQ ID No. 23 |
| LC70 | TCGTTCCTATGACTCCACCA RGA SEQ ID No. 24 |
| LC73 | CACCATGGCTGCGAGCGATGAAGT GID1a SEQ ID No. 25 |
| LC74 | ACATTCCGCGTTTACAAACGC GID1a SEQ ID No. 26 |
| LC75 | CACCCTAGATCCAAGATCAGACC RGA SEQ ID No. 27 |
| LC76 | GTACGCCGCCGTCGAGAGT RGA SEQ ID No. 28 |
| LC77 | GAGATGGCGGAGGTTGCTTTGAGACTCGAACAATTAG RGA |
| | SEQ ID No. 29 |
| LC78 | CTAATTGTTCGAGTCTCAAAGCAACCTCCGCCATCTC RGA |
| | SEQ ID No. 30 |
| LC80 | CACCATGAAGAGAGATCATCATC GAI SEQ ID No. 31 |
| LC81 | ATTGGTGGAGAGTTTCCAAG GAI SEQ ID No. 32 |
| LC85 | GCCTCAAAAGACACCTCTGG OTS2 SEQ ID No. 33 |
| LC86 | GCTTATCCAGCTTCCACGTC OTS2 SEQ ID No. 34 |
| lcm26 | CCGTCGGAGCTTTATTCTTGG RGA SEQ ID No. 35 |
| lcm27 | CGTCGTTCCTATGACTCCACC RGA SEQ ID No. 36 |
| lcm28 | GCAAAACCTAGATCCGACATTG GAI SEQ ID No. 37 |
| lcm29 | GCTCCGCCGGATTATAGTG GAI SEQ ID No. 38 |
| mr37 | CTCTCCCGCTATGTATGTCGCCA ACT2 SEQ ID No. 39 |
| mr38 | GTGAGACACACCATCACCAG ACT2 SEQ ID No. 40 |

### Sequence Listing

| SEQ ID No. 1 | |
|---|---|
| Gai nucleic acid sequence: | |
| 1 | ataaccttcc tctctatttt tacaatttat tttgttatta gaagtggtag tggagtgaaa |
| 61 | aaacaaatcc taagcagtcc taaccgatcc ccgaagctaa agattcttca ccttcccaaa |
| 121 | taaagcaaaa cctagatccg acattgaagg aaaaaccttt tagatccatc tctgaaaaaa |
| 181 | aaccaaccat gaagagagat catcatcatc atcatcatca agataagaag actatgatga |
| 241 | tgaatgaaga agacgacggt aacggcatgg atgagcttct agctgttctt ggttacaagg |
| 301 | ttaggtcatc cgaaatggct gatgttgctc agaaactcga gcagcttgaa gttatgatgt |
| 361 | ctaatgttca agaagacgat ctttctcaac tcgctactga gactgttcac tataatccgg |
| 421 | cggagcttta cacgtggctt gattctatgc tcaccgacct taatcctccg tcgtctaacg |
| 481 | ccgagtacga tcttaaagct attcccggtg acgcgattct caatcagttc gctatcgatt |
| 541 | cggcttcttc gtctaaccaa ggcggcggag gagatacgta tactacaaac aagcggttga |
| 601 | aatgctcaaa cggcgtcgtg gaaaccacta cagcgacggc tgagtcaact cggcatgttg |
| 661 | tcctggttga ctcgcaggag aacggtgtgc gtctcgttca cgcgcttttg gcttgcgctg |
| 721 | aagctgttca gaaagagaat ctgactgtag cggaagctct ggtgaagcaa atcggattct |
| 781 | tagccgtttc tcaaatcgga gcgatgagaa aagtcgctac ttacttcgcc gaagctctcg |
| 841 | cgcggcggat ttaccgtctc tctccgtcgc agagtccaat cgaccactct ctctccgata |
| 901 | ctcttcagat gcacttctac gagacttgtc cttatctcaa gttcgctcac ttcacggcga |
| 961 | atcaagcgat tctcgaagct tttcaaggga agaaaagagt tcatgtcatt gatttctcta |
| 1021 | tgagtcaagg tcttcaatgg ccggcgctta tgcaggctct tgcgcttcga cctggtggtc |
| 1081 | ctcctgtttt ccggttaacc ggaattggtc caccggcacc ggataatttc gattatcttc |
| 1141 | atgaagttgg gtgtaagctg gctcatttag ctgaggcgat tcacgttgag tttgagtaca |
| 1201 | gaggatttgt ggctaacact ttagctgatc ttgatgcttc gatgcttgag cttagaccaa |
| 1261 | gtgagattga atctgttgcg gttaactctg ttttcgagct tcacaagctc ttgggacgac |
| 1321 | ctggtgcgat cgataaggtt cttggtgtgg tgaatcagat taaaccggag attttcactg |
| 1381 | tggttgagca ggaatcgaac cataatagtc cgattttctt agatcggttt actgagtcgt |
| 1441 | tgcattatta ctcgacgttg tttgactcgt tggaaggtgt accgagtggt caagacaagg |
| 1501 | tcatgtcgga ggtttacttg ggtaaacaga tctgcaacgt tgtggcttgt gatggacctg |
| 1561 | accgagttga gcgtcatgaa acgttgagtc agtggaggaa ccggttcggg tctgctgggt |
| 1621 | ttgcggctgc acatattggt tcgaatgcgt ttaagcaagc gagtatgctt ttggctctgt |
| 1681 | tcaacggcgg tgagggttat cgggtggagg agagtgacgg ctgtctcatg ttgggttggc |
| 1741 | acacacgacc gctcatagcc acctcggctt ggaaactctc caccaattag atggtggctc |
| 1801 | aatgaattga tctgttgaac cggttatgat gatagatttc cgaccgaagc caaactaaat |
| 1861 | cctactgttt ttccctttgt cacttgttaa gatcttatct ttcattatat taggtaattg |
| 1921 | aaaaatttta atctcgcttt ggagagtttt ttttttttgc atgtgacatt ggagggtaaa |
| 1981 | ttggataggc agaaatagaa gtatgtgtta ccaagtatgt gcaattggtt gaaataaaat |
| 2041 | catcttgagt gtcaccatct ataaaattca ttgtaatgac taatgagcct gattaaactg |
| 2101 | tctcttatga taatgtgctg attctcatg |
| SEQ ID No. 2 | |
| GAI peptide sequence: | |
| MKRDHHHHHHQDKKTMMMNEEDDGNGMDELLAVLGYKVRSSEMA | |
| DVAQKLEQLEVMMSNVQEDDLSQLATETVHYNPAELYTWLDSMLTDLNPPS | |
| SNAEYDLKAIPGDAILNQFAIDSASSSNQGGGGDTYTTNKRLKCSNGVVETTT | |
| ATAESTRHVVLVDSQENGVRLVHALLACAEAVQKENLTVAEALVKQIGFLA | |
| VSQIGAMRKVATYFAEALARRIYRLSPSQSPIDHSLSDTLQMHFYETCPYLKF | |
| AHFTANQAILEAFQGKKRVHVIDFSMSQGLQWPALMQALALRPGGPPVFRL | |
| TGIGPPAPDNFDYLHEVGCKLAHLAEAIHVEFEYRGFVANTLADLDASMLEL | |
| RPSEIESVAVNSVFELHKLLGRPGAIDKVLGVVNQIKPEIFTVVEQESNHNSPIF | |
| LDRFTESLHYYSTLFDSLEGVPSGQDKVMSEVYLGKQICNVVACDGPDRVER | |
| HETLSQWRNRFGSAGFAAAHIGSNAFKQASMLLALFNGGEGYRVEESDGCL | |
| MLGWHTRPLIATSAWKLSTN | |
| SEQ ID No. 3 | |
| rga nucleic acid sequence: | |
| 1 | atgaatgatg attgaagtgg tagtagcagt gaaaaacaaa agcaatccaa tcccaaaccc |
| 61 | atttgctctt aagattcttc acatagagaa gtcacatgtt ccttcttctt cttccttcat |
| 121 | catccccaaa cacacacaaa ctaaaaaaaa ggcaaaaccc tagatccaag atcagaccta |
| 181 | atctaatcga aactcatagc tgaaaaatga agagagatca tcaccaattc caaggtcgat |
| 241 | tgtccaacca cgggacttct tcttcatcat catcaatctc taaagataag atgatgatgg |
| 301 | tgaaaaaaga agaagacggt ggaggtaaca tggacgacga gcttctcgct gttttaggtt |
| 361 | acaaagttag gtcatcggag atggcggagg ttgctttgaa actcgaacaa ttagagacga |
| 421 | tgatgagtaa tgttcaagaa gatggtttat ctcatctcgc gacggatact gttcattata |
| 481 | atccgtcgga gctttattct tggcttgata atatgctctc tgagcttaat cctcctcctc |
| 541 | ttccggcgag ttctaacggt ttagatccgg ttcttccttc gccggagatt tgtggttttc |
| 601 | cggcttcgga ttatgacctt aaagtcattc ccggaaacgc gatttatcag tttccggcga |
| 661 | ttgattcttc gtcttcgtcg aataatcaga acaagcgttt gaaatcatgc tcgagtcctg |
| 721 | attctatggt tacatcgact tcgacgggta cgcagattgg tggagtcata ggaacgacgg |
| 781 | tgacgacaac caccacgaca acgacggcgg cgggtgagtc aactcgttct gttatcctgg |
| 841 | ttgactcgca agagaacggt gttcgtttag tccacgcgct tatggcttgt gcagaagcaa |
| 901 | tccagcagaa caatttgact ctagcggaag ctcttgtgaa gcaaatcgga tgcttagctg |
| 961 | tgtctcaagc cggagctatg agaaaagtgg ctacttactt cgccgaagct ttagcgcggc |
| 1021 | ggatctaccg tctctctccg ccgcagaatc agatcgatca ttgtctctcc gatactcttc |
| 1081 | agatgcactt ttacgagact tgtccttatc ttaaattcgc tcacttcacg gcgaaccaag |
| 1141 | cgattctcga agcttttgaa ggtaagaaga gagtacacgt cattgatttc tcgatgaacc |
| 1201 | aaggtcttca atggcctgca cttatgcaag ctcttgcgct tcgagaagga ggtcctccaa |
| 1261 | ctttccggtt aaccggaatt ggtccaccgg cgccggataa ttctgatcat cttcatgaag |
| 1321 | ttggttgtaa attagctcag cttgcggagg cgattcacgt agaattcgaa taccgtggat |
| 1381 | tcgttgctaa cagcttagcc gatctcgatg cttcgatgct tgagcttaga ccgagcgata |
| 1441 | cggaagctgt tgcggtgaac tctgtttttg agctacataa gctcttaggt cgtcccggtg |
| 1501 | ggatagagaa agttctcggc gttgtgaaac agattaaacc ggtgattttc acggtggttg |
| 1561 | agcaagaatc gaaccataac ggaccggttt tcttagaccg gtttactgaa tcgttacatt |
| 1621 | attattcgac tctgtttgat tcgttggaag gagttccgaa tagtcaagac aaagtcatgt |
| 1681 | ctgaagttta cttagggaaa cagatttgta atctggtggc ttgtgaaggt cctgacagag |
| 1741 | tcgagagaca cgaaacgttg agtcaatggg gaaaccggtt tggttcgtcc ggtttagcgc |
| 1801 | cggcacatct tgggtctaac gcgtttaagc aagcgagtat gcttttgtct gtgtttaata |
| 1861 | gtggccaagg ttatcgtgtg gaggagagta atggatgttt gatgttgggt tggcacactc |
| 1921 | gtccactcat taccacctcc gcttggaaac tctcgacggc ggcgtactga gtttgactcg |
| 1981 | aagcatacga cggtggtgga gtcgagtcga gtgaatttga gattgagatc agtggaccgg |
| 2041 | tgatgacata tgttcggacc aagacctaaa ccgaactgaa tcgaaccgtt ttgccttttg |
| 2101 | tttattttat ttattttcgt tcacttgttt aaaattctta tatatatcgt tttggtaggt |
| 2161 | catttttaat ttatgccttt ttgggatcaa tttttaatag gctgagtttg tatttattaa |
| 2221 | taaattatct ttatgaattt taaactaaaa ctatgtttta atctcattta aaaaaaaatt |
| 2281 | aatatcaagt tttattaatc tc |
| SEQ ID No. 4 | |
| RGA peptide sequence: | |
| MKRDHHQFQGRLSNHGTSSSSSSISKDKMMMVKKEEDGGGNMDDELLAVL | |
| GYKVRSSEMAEVALKLEQLETMMSNVQEDGLSHLATDTVHYNPSELYSWL | |
| DNMLSELNPPPLPASSNGLDPVLPSPEICGFPASDYDLKVIPGNAIYQFPAIDSS | |
| SSSNNQNKRLKSCSSPDSMVTSTSTGTQIGGVIGTTVTTTTTTTTAAGESTRSV | |
| ILVDSQENGVRLVHALMACAEAIQQNNLTLAEALVKQIGCLAVSQAGAMRK | |
| VATYFAEALARRIYRLSPPQNQIDHCLSDTLQMHFYETCPYLKFAHFTANQAI | |
| LEAFEGKKRVHVIDFSMNQGLQWPALMQALALREGGPPTFRLTGIGPPAPDN | |
| SDHLHEVGCKLAQLAEAIHVEFEYRGFVANSLADLDASMLELRPSDTEAVAV | |
| NSVFELHKLLGRPGGIEKVLGVVKQIKPVIFTVVEQESNHNGPVFLDRFTESL | |
| HYYSTLFDSLEGVPNSQDKVMSEVYLGKQICNLVACEGPDRVERHETLSQW | |
| GNRFGSSGLAPAHLGSNAFKQASMLLSVFNSGQGYRVEESNGCLMLGWHTR | |
| PLITTSAWKLSTAAY | |
| SEQ ID No. 5 | |
| rgl-1 nucleic acid sequence: | |
| ATATCATTATTTAAAAATAGAATTTTATTTTTCTTTCTTCTTCTTCAATTATTATGACA | |
| CTCCCGTGTTCCTAATCTTTTCTCTTATTCTTCTCTTTCTTCTCATCTTACAAAATCTTG | |
| CAAATCAATTTTAATGAAGAGAGAGCACAACCACCGTGAATCATCCGCCGGAGAAG | |
| GTGGGAGTTCATCAATGACGACGGTGATTAAAGAAGAAGCTGCCGGAGTTGACGAG | |
| CTTTTGGTTGTTTTAGGTTACAAAGTTCGATCATCCGACATGGCTGACGTGGCACAC | |
| AAGCTTGAACAGTTAGAGATGGTTCTTGGTGATGGAATCTCGAATCTTTCTGATGAA | |
| ACTGTTCATTACAATCCTTCTGATCTCTCTGGTTGGGTCGAAAGCATGCTCTCGGATC | |
| TTGACCCGACCCGGATTCAAGAAAAGCCTGACTCAGAGTACGATCTTAGAGCTATTC | |
| CTGGCTCTGCAGTGTATCCACGTGACGAGCACGTGACTCGTCGGAGCAAGAGGACG | |
| AGAATTGAATCGGAGTTATCCTCTACGCGCTCTGTGGTGGTTTTGGATTCTCAAGAA | |
| ACTGGAGTGCGTTTAGTCCACGCGCTATTAGCTTGTGCTGAAGCTGTTCAACAGAAC | |
| AATTTGAAGTTAGCCGACGCGCTCGTGAAGCACGTGGGGTTACTCGCGTCCTCTCAA | |
| GCTGGTGCTATGAGGAAAGTCGCGACTTACTTCGCTGAAGGGCTTGCGAGAAGGAT | |
| TTACCGTATTTACCCTCGAGACGATGTCGCGTTGTCTTCGTTTTCGGACACTCTTCAG | |
| ATTCATTTCTATGAGTCTTGTCCGTATCTCAAGTTTGCGCATTTTACGGCGAATCAAG | |
| CGATACTTGAGGTTTTTGCTACGGCGGAGAAGGTTCATGTTATTGATTTAGGACTTA | |
| ACCATGGTTTACAATGGCCGGCTTTGATTCAAGCTCTTGCTTTACGTCCTAATGGTCC | |
| ACCGGATTTTCGGTTAACCGGGATCGGTTATTCGTTAACCGATATTCAAGAAGTTGG | |
| TTGGAAACTTGGTCAGCTTGCGAGTACTATTGGTGTCAATTTCGAATTCAAGAGCAT | |
| TGCTTTAAACAATTTGTCTGATCTTAAACCGGAAATGCTAGACATTAGACCCGGTTT | |
| AGAATCAGTGGCGGTTAACTCGGTCTTCGAGCTTCATCGCCTCTTAGCTCATCCCGG | |
| TTCCATCGATAAGTTTTTATCGACAATCAAATCAATCCGACCGGATATAATGACTGT | |
| GGTCGAGCAAGAAGCAAACCATAACGGTACCGTATTTCTCGATCGGTTCACGGAAT | |
| CGCTACATTACTATTCGAGCTTATTCGACTCGCTCGAGGGCCCGCCAAGCCAAGACC | |
| GAGTGATGTCGGAGTTATTCCTAGGACGGCAGATACTAAACCTTGTGGCATGCGAA | |
| GGAGAAGACCGGGTAGAGAGGCATGAGACTTTAAATCAGTGGAGAAACCGGTTCGG | |
| TTTAGGAGGATTTAAACCGGTTAGTATCGGTTCGAACGCGTATAAGCAAGCAAGCAT | |
| GTTGTTGGCACTTTATGCCGGGGCTGATGGGTATAATGTGGAAGAGAATGAAGGTTG | |
| TTTGTTGCTTGGATGGCAAACGCGACCGCTTATTGCAACATCTGCGTGGCGAATCAA | |
| TCGTGTGGAATAAAAATAAATAATGGGAAAAGTGAAAATGTGCTATATACTTTATTG | |
| CATTGCTGATAAAGAAAAAAAGTCCCACGTTTTCCAAATTTTATGAATTCTAAATTT | |
| TGTTCACTTGTCACGAGATTTTGACCTCGCATAAATAGACTATTACGTCAGGGTCAG | |
| GCCAATGAAATGATTTTTTATCA | |
| SEQ ID No. 6 | |
| RGL-1 peptide sequence: | |
| MKREHNHRESSAGEGGSSSMTTVIKEEAAGVDELLVVLGYKVRSSDMADVAHKLE | |
| QLEMVLGDGISNLSDETVHYNPSDLSGWVESMLSDLDPTRIQEKPDSEYDLRAIPGSA | |
| VYPRDEHVTRRSKRTRIESELSSTRSVVVLDSQETGVRLVHALLACAEAVQQNNLKL | |
| ADALVKHVGLLASSQAGAMRKVATYFAEGLARRIYRIYPRDDVALSSFSDTLQIHFY | |
| ESCPYLKFAHFTANQAILEVFATAEKVHVIDLGLNHGLQWPALIQALALRPNGPPDF | |
| RLTGIGYSLTDIQEVGWKLGQLASTIGVNFEFKSIALNNLSDLKPEMLDIRPGLESVA | |
| VNSVFELHRLLAHPGSIDKFLSTIKSIRPDIMTVVEQEANHNGTVFLDRFTESLHYYSS | |
| LFDSLEGPPSQDRVMSELFLGRQILNLVACEGEDRVERHETLNQWRNRFGLGGFKPV | |
| SIGSNAYKQASMLLALYAGADGYNVEENEGCLLLGWQTRPLIATSAWRINRVE” | |
| SEQ ID No. 7 | |
| RGL-2 nucleic acid sequence: | |
| 1 | caaatcccat taataaaaac cttaccaacc catgaagtaa agtaaactcc tttcttataa |
| 61 | actctctttt gttctttttt tttcaacttc atcagtctct taactcacca tcacaagaac |
| 121 | aagaaagatg aagagaggat acggagaaac atgggatccg ccaccaaaac cactaccagc |
| 181 | ttctcgttcc ggagaaggtc cttcaatggc ggataagaag aaggctgatg atgacaacaa |
| 241 | caacagcaac atggatgatg agcttcttgc tgttcttggc tacaaggttc gatcttctga |
| 301 | gatggctgaa gtagcacaga agcttgaaca acttgagatg gtcttgtcta atgatgatgt |
| 361 | tggttctact gtcttaaacg actctgttca ttataaccca tctgatctct ctaactgggt |
| 421 | cgagagcatg ctttctgagc tgaacaaccc ggcttcttcg gatcttgaca cgacccgaag |
| 481 | ttgtgtggat agatccgaat acgatctcag agcaattccg ggtctttctg cgtttccaaa |
| 541 | ggaagaggaa gtctttgacg aggaagctag cagcaagagg atccgactcg gatcgtggtg |
| 601 | cgaatcgtcg gacgagtcaa ctcggtccgt ggtgctcgtt gactctcagg agaccggagt |
| 661 | tagacttgtc cacgcactag tggcgtgcgc tgaggcgatt caccaggaga atctcaactt |
| 721 | agctgacgcg ctggtgaaac gcgtgggaac actcgcgggt tctcaagctg gagctatggg |
| 781 | aaaagtcgct acgtattttg ctcaagcctt ggctcgtcgt atttaccgtg attacacggc |
| 841 | ggagacggac gtttgcgcgg cggtgaaccc atctttcgaa gaggttttgg agatgcactt |
| 901 | ttacgagtct tgcccttacc tgaagttcgc tcatttcacg gcgaaccaag cgattctaga |
| 961 | agctgttacg acggcgcgta gagttcacgt cattgattta gggcttaatc aagggatgca |
| 1021 | atggcctgct ttaatgcaag ctttagctct ccgacccggt ggacctccgt cgtttcgtct |
| 1081 | caccggaatc ggaccaccgc agacggagaa ttcagattcg cttcaacagt taggttggaa |
| 1141 | attagctcaa ttcgctcaga acatgggcgt tgaattcgaa ttcaaaggct tagccgctga |
| 1201 | gagtttatcg gatcttgaac ccgaaatgtt cgaaacccga cccgaatctg aaaccttagt |
| 1261 | ggttaattcg gtatttgagc tccaccggtt attagcccga tccggttcaa tcgaaaagct |
| 1321 | tctcaatacg gttaaagcta ttaaaccgag tatcgtaacg gtggttgagc aagaagcgaa |
| 1381 | ccacaacgga atcgtcttcc tcgataggtt caacgaagcg cttcattact actcgagctt |
| 1441 | gtttgactcg ctcgaagaca gttatagttt accgagtcaa gaccgagtta tgtcagaagt |
| 1501 | gtacttaggg agacagatac tcaacgttgt tgcggcggaa gggtccgatc gggtcgagcg |
| 1561 | gcacgagacg gctgcacagt ggaggattcg gatgaaatcc gctgggtttg acccgattca |
| 1621 | tctcggatct agcgcgttta aacaagcgag tatgctttta tcgctttacg ctaccggaga |
| 1681 | tggatacaga gttgaagaaa atgacggatg tttaatgata gggtggcaga cgcgaccact |
| 1741 | catcacaacc tcggcgtgga aactcgcctg agtcgcggcg gtagagatga ctcgcctgaa |
| 1801 | accgggaaaa acaataaatg ttttaaaaaa ttaggaaaag agaccgtaac tttagttatg |
| 1861 | tttttacttt ttaacccgaa gtttttgtgt gtttaacctt tttgcctaaa tgtttacaac |
| 1921 | tttatctttt tggaccttgt gcgtatcttt gagagttaag agaacgagta aaaaatcttg |
| 1981 | tatcgtagat cgagctaagt agttttcaat aaatggaagg ataacgattc tgtatgtttt |
| 2041 | ttacttgatc caatatatat gaatttattt |
| SEQ ID No. 8 | |
| RGL-2 peptide sequence: | |
| MKRGYGETWDPPPKPLPASRSGEGPSMADKKKADDDNNNSNMDDELLAVL | |
| GYKVRSSEMAEVAQKLEQLEMVLSNDDVGSTVLNDSVHYNPSDLSNWVES | |
| MLSELNNPASSDLDTTRSCVDRSEYDLRAIPGLSAFPKEEEVFDEEASSKRIRL | |
| GSWCESSDESTRSVVLVDSQETGVRLVHALVACAEAIHQENLNLADALVKR | |
| VGTLAGSQAGAMGKVATYFAQALARRIYRDYTAETDVCAAVNPSFEEVLEM | |
| HFYESCPYLKFAHFTANQAILEAVTTARRVHVIDLGLNQGMQWPALMQALA | |
| LRPGGPPSFRLTGIGPPQTENSDSLQQLGWKLAQFAQNMGVEFEFKGLAAES | |
| LSDLEPEMFETRPESETLVVNSVFELHRLLARSGSIEKLLNTVKAIKPSIVTVV | |
| EQEANHNGIVFLDRFNEALHYYSSLFDSLEDSYSLPSQDRVMSEVYLGRQILN | |
| VVAAEGSDRVERHETAAQWRIRMKSAGFDPIHLGSSAFKQASMLLSLYATG | |
| DGYRVEENDGCLMIGWQTRPLITTSAWKLA | |
| SEQ ID No. 9 | |
| AtGID1a nucleic acid sequence: | |
| 1 | gtttttaatc actcaaccat taaaccccat tttgatctct agttttttaa aagcaggaga |
| 61 | ttttcctttt cccagaaaag aaatttccca aatcaaagtt tcgagctttc acttctcgac |
| 121 | ttgcaaattc tcgtcctttt tactgaattc gatctgggtt tttgtttttg attagtaaaa |
| 181 | taacaaaaaa aaaaaaaagg atttatcaga aatggctgcg agcgatgaag ttaatcttat |
| 241 | tgagagcaga acagtggttc ctctcaatac atgggtttta atatccaact tcaaagtagc |
| 301 | ctacaatatc cttcgtcgcc ctgatggaac ctttaaccga cacttagctg agtatctaga |
| 361 | ccgtaaagtc actgcaaacg ccaatccggt tgatggggtt ttctcgttcg atgtcttgat |
| 421 | tgatcgcagg atcaatcttc taagcagagt ctatagacca gcttatgcag atcaagagca |
| 481 | acctcctagt attttagatc tcgagaagcc tgttgatggc gacattgtcc ctgttatatt |
| 541 | gttcttccat ggaggtagct ttgctcattc ttctgcaaac agtgccatct acgatactct |
| 601 | ttgtcgcagg cttgttggtt tgtgcaagtg tgttgttgtc tctgtgaatt atcggcgtgc |
| 661 | accagagaat ccataccctt gtgcttatga tgatggttgg attgctctta attgggttaa |
| 721 | ctcgagatct tggcttaaat ccaagaaaga ctcaaaggtc catattttct tggctggtga |
| 781 | tagctctgga ggtaacatcg cgcataatgt ggctttaaga gcgggtgaat cgggaatcga |
| 841 | tgttttgggg aacattctgc tgaatcctat gtttggtggg aatgagagaa cggagtctga |
| 901 | gaaaagtttg gatgggaaat actttgtgac ggttagagac cgcgattggt actggaaagc |
| 961 | gtttttaccc gagggagaag atagagagca tccagcgtgt aatccgttta gcccgagagg |
| 1021 | gaaaagctta gaaggagtga gtttccccaa gagtcttgtg gttgtcgcgg gtttggattt |
| 1081 | gattagagat tggcagttgg catacgcgga agggctcaag aaagcgggtc aagaggttaa |
| 1141 | gcttatgcat ttagagaaag caactgttgg gttttacetc ttgcctaata acaatcattt |
| 1201 | ccataatgtt atggatgaga tttcggcgtt tgtaaacgcg gaatgttaac actgggttag |
| 1261 | agaaagaagg ttgttttaac aaagccaaga catctttcaa actaacacac aggtgaatgt |
| 1321 | attgcctgtg gattctctcg tttagttttg tttttgtgtt tagtatctaa gtgtgtggcg |
| 1381 | gtctgcggca gcctttgtga tgactgttta aacgctggat tctgaaacgc taaagcttgt |
| 1441 | ggaagaacag tgaggcgttt agagacttgg aaaggaacca agcactagta aaaatttctc |
| 1501 | ctttttttgt ctgtaatatt tggcatttag cttttaccct tgagcctttt tactaactaa |
| 1561 | aagctgattt tttcagcatg agagtggtaa ttagatatct ataaatatat atatttcaag |
| 1621 | aatgtaatgt ttatacacaa attttagtga ttttggtaaa tgtatgtagg gtctgcactc |
| 1681 | tgcagttgta ttgttgctcc tctttttcat tgtactctaa tggattttac aaaaataagc |
| SEQ ID No. 10 | |
| AtGID1 peptide sequence: | |
| MAASDEVNLIESRTVVPLNTWVLISNFKVAYNILRRPDGTFNRHLAEYLDRK | |
| VTANANPVDGVFSFDVLIDRRINLLSRVYRPAYADQEQPPSILDLEKPVDGDI | |
| VPVILFFHGGSFAHSSANSAIYDTLCRRLVGLCKCVVVSVNYRRAPENPYPCA | |
| YDDGWIALNWVNSRSWLKSKKDSKVHIFLAGDSSGGNIAHNVALRAGESGI | |
| DVLGNILLNPMFGGNERTESEKSLDGKYFVTVRDRDWYWKAFLPEGEDREH | |
| PACNPFSPRGKSLEGVSFPKSLVVVAGLDLIRDWQLAYAEGLKKAGQEVKL | |
| MHLEKATVGFYLLPNNNHFHNVMDEISAFVNAEC | |
| SEQ ID No. 11 | |
| RGL-3 nucleic acid sequence: | |
| 1 | acatgcgaaa ttataatggc ctgcctctct tccttcttat ctcttttact tacactctcc |
| 61 | aggtccctca cttccctcat tggacctctc taactctcct ctcttacctt ctcctgttta |
| 121 | aattcttctc ctttctttcc acaatttctg tctaaccaat tccaacacca aaaaattcca |
| 181 | tttcttgacg atgaaacgaa gccatcaaga aacgtctgta gaagaagaag ctccttcaat |
| 241 | ggtggagaag ttagaaaatg gttgtggtgg tggtggagac gataacatgg acgagtttct |
| 301 | tgctgttttg ggttacaagg ttcgatcttc agacatggca gatgttgcac agaagcttga |
| 361 | acagcttgaa atggtcttgt ccaatgatat tgcctcttct agtaatgcct tcaatgacac |
| 421 | cgttcattac aatccttctg atctctccgg ttgggctcag agcatgctct cggatcttaa |
| 481 | ttactacccg gatcttgacc cgaaccggat ttgcgatctg agaccaatca cagacgacga |
| 541 | tgagtgttgc agtagcaata gtaacagcaa caagaggatt cgactcggtc cttggtgtga |
| 601 | ctcagtgacc agcgagtcaa ctcgttccgt ggtgcttatc gaggagacag gagttagact |
| 661 | cgttcaggcg ctagtggcct gcgccgaggc ggttcagctg gagaatctga gcctcgcgga |
| 721 | tgctctcgtc aagcgcgtgg gattactcgc ggcttctcaa gccggagcca tggggaaagt |
| 781 | cgctacctac ttcgccgaag ccctagctcg tcgaatttac cggattcatc cttccgccgc |
| 841 | cgccattgat ccttccttcg aagagattct tcagatgaac ttctacgact cgtgtcccta |
| 901 | cctgaaattc gctcatttca cggccaatca ggcgattcta gaagctgtta cgacgtcgcg |
| 961 | tgtcgtacac gtaatcgatc tagggcttaa tcaaggtatg caatggccgg cgttaatgca |
| 1021 | agccttagct ctccgacccg gtggtccacc gtcgtttcgt ctcagtggcg ttgggaatcc |
| 1081 | gtcgaatcga gaagggattc aagagttagg ttggaagcta gctcagctgg ctcaagccat |
| 1141 | cggcgtcgaa ttcaaattca atggtctaac gacggagagg ttatccgatt tagaaccgga |
| 1201 | tatgttcgag acccgaaccg aatcggagac tctagtggtt aattcggttt tcgagcttca |
| 1261 | cccggtttta tcccaacccg gttcgatcga aaagctgtta gcgacggtta aggcggttaa |
| 1321 | accgggtctc gtaacagtgg tggaacaaga agcgaaccat aacggtgacg ttttcttaga |
| 1381 | ccggtttaac gaagcgcttc actattactc gagcttgttc gactcgctcg aagatggtgt |
| 1441 | tgtgataccg agtcaagacc gagtcatgtc ggaggtttac ttagggagac agatattgaa |
| 1501 | cttggtggcg acggaaggaa gcgataggat cgagcgacac gagacgctgg ctcagtggcg |
| 1561 | aaaacgtatg ggatccgccg ggtttgaccc ggttaacctc ggatcagacg cgtttaagca |
| 1621 | agcgagtttg ctattggcgt tatctggcgg tggagatgga tacagagtgg aggagaacga |
| 1681 | cggaagccta atgcttgcgt ggcaaacgaa acctctaatc gctgcatcgg cgtggaaact |
| 1741 | agcggcggag ttgcggcggt agatacgtcg tcataaagag gagaagaaaa aagacttagc |
| 1801 | gaacgtgacc ttatgttttt attttacttt aacttacccc agtagtttcg ttttgtgaca |
| 1861 | atttcgcccg aaatattccg tgccttatac ttttgggacc cagttggttc gttggtcgtg |
| 1921 | gagattcgag aacgaggaac atgtgtgtat gtaacaacag cacgagcaag tgttttcata |
| 1981 | gtttgaataa atatgaaaga aatgacgttt atttt |

SEQ ID No. 12
RGL-3 peptide sequence:
MKRSHQETSVEEEAPSMVEKLENGCGGGGDDNMDEFLAVLGYKVRSSDMA
DVAQKLEQLEMVLSNDIASSSNAFNDTVHYNPSDLSGWAQSMLSDLNYYPD
LDPNRICDLRPITDDDECCSSNSNSNKRIRLGPWCDSVTSESTRSVVLIEETGV
RLVQALVACAEAVQLENLSLADALVKRVGLLAASQAGAMGKVATYFAEAL
ARRIYRIHPSAAAIDPSFEEILQMNFYDSCPYLKFAHFTANQAILEAVTTSRVV
HVIDLGLNQGMQWPALMQALALRPGGPPSFRLTGVGNPSNREGIQELGWKL
AQLAQAIGVEFKFNGLTTERLSDLEPDMFETRTESETLVVNSVFELHPVLSQP
GSIEKLLATVKAVKPGLVTVVEQEANHNGDVFLDRFNEALHYYSSLFDSLED
GVVIPSQDRVMSEVYLGRQILNLVATEGSDRIERHETLAQWRKRMGSAGFDP
VNLGSDAFKQASLLLALSGGGDGYRVEENDGSLMLAWQTKPLIAASAWKLA
AELRR
SEQ ID No. 13
AtARF19 nucleic acid sequence
ATGAAAGCTCCATCAAATGGATTTCTTCCAAGTTCCAACGAAGGAGAGAA
GAAGCCAATCAATTCTCAACTATGGCACGCTTGTGCAGGGCCTTTAGTTTC
ATTACCTCCTGTGGGAAGTCTTGTGGTTTACTTCCCTCAAGGACACAGCGA
GCAAGTTGCAGCATCGATGCAGAAGCAAACAGATTTTATACCAAATTACC
CAAATCTTCCTTCTAAGCTGATTTGCTTGCTTCACAGTGTTACATTACATG
CTGATACCGAAACAGATGAAGTCTATGCACAAATGACTCTTCAACCTGTG
AATAAGTATGATAGAGAAGCATTGCTAGCTTCTGATATGGGCTTGAAGCT
AAACAGACAACCTACTGAGTTTTTTTGCAAGACTCTTACTGCAAGTGACA
CAAGCACTCATGGTGGATTCTCTGTACCGCGTCGTGCAGCTGAGAAAATA
TTCCCTCCTCTTGATTTCTCGATGCAACCGCCTGCGCAAGAGATTGTAGCT
AAAGATTTACATGATACTACATGGACTTTCAGACATATCTATCGAGGCCA
ACCAAAAAGACACTTGCTTACCACAGGTTGGAGCGTTTTTGTTAGCACAA
AGAGACTATTTGCGGGTGATTCAGTTTTGTTTGTAAGAGATGAGAAATCA
CAGCTGATGTTGGGTATAAGACGTGCAAATAGACAAACTCCGACTCTTTC
CTCATCGGTCATATCCAGCGACAGTATGCACATTGGGATACTTGCAGCTG
CAGCTCATGCTAATGCCAATAGTAGCCCTTTTACCATCTTCTTCAATCCAA
GGGCAAGTCCTTCAGAGTTTGTAGTTCCTTTAGCCAAATACAACAAAGCC
TTATACGCTCAAGTATCTCTAGGAATGAGATTCCGGATGATGTTTGAGACT
GAGGATTGTGGGGTTCGTAGATATATGGGTACAGTCACAGGTATTAGTGA
TCTTGACCCTGTAAGATGGAAAGGCTCACAATGGCGTAATCTTCAGGTAG
GATGGGATGAATCAACAGCTGGAGATAGGCCAAGCCGAGTATCCATATG
GGAAATCGAACCCGTCATAACTCCTTTTTACATATGTCCTCCTCCATTTTT
CAGACCTAAGTACCCGAGGCAACCCGGGATGCCAGATGATGAGTTAGAC
ATGGAAAATGCTTTCAAAAGAGCAATGCCTTGGATGGGAGAAGACTTTGG
GATGAAGGACGCACAGAGTTCGATGTTCCCTGGTTTAAGTCTAGTTCAAT
GGATGAGTATGCAGCAAAACAATCCATTGTCAGGTTCTGCTACTCCTCAG
CTCCCGTCCGCGCTCTCATCTTTTAACCTACCAAACAATTTTGCTTCCAAC
GACCCTTCCAAGCTGTTGAACTTCCAATCCCCAAACCTCTCTTCCGCAAAT
TCCCAATTCAACAAACCGAACACGGTTAACCATATCAGCCAACAGATGCA
AGCACAACCAGCCATGGTGAAATCTCAACAACAACAACAACAACAACAA
CAACAACACCAACACCAACAACAACAACTGCAACAACAACAACAACTAC
AGATGTCACAGCAACAGGTGCAGCAACAAGGGATTTATAACAATGGTAC
GATTGCTGTTGCTAACCAAGTCTCTTGTCAAAGTCCAAACCAACCTACTGG
ATTCTCTCAGTCTCAGCTTCAGCAGCAGTCAATGCTCCCTACTGGTGCTAA
AATGACACACCAGAACATAAATTCTATGGGGAATAAAGGCTTGTCTCAAA
TGACATCGTTTGCGCAAGAAATGCAGTTTCAGCAGCAACTGGAAATGCAT
AACAGTAGCCAGTTATTAAGAAACCAGCAAGAACAGTCCTCTCTCCATTC
ATTACAACAAAATCTGTCCCAAAATCCTCAGCAACTCCAAATGCAACAAC
AATCATCAAAACCAAGTCCTTCACAACAGCTTCAGTTGCAGCTACTGCAG
AAGCTACAGCAGCAGCAACAGCAGCAGTCGATTCCTCCAGTAAGCTCATC
CTTACAGCCACAATTATCAGCGTTGCAGCAGACACAAAGCCATCAATTGC
AACAACTTCTGTCGTCTCAAAATCAACAGCCCTTGGCACATGGTAATAAC
AGCTTCCCAGCTTCAACTTTCATGCAGCCTCCACAGATTCAGGTGAGTCCT
CAGCAGCAAGGACAGATGAGTAACAAAAATCTTGTAGCCGCTGGAAGAT
CACATTCTGGCCACACAGATGGAGAAGCTCCTTCTTGTTCAACCTCACCTT
CCGCCAATAACACGGGACATGATAATGTTTCACCGACAAATTTCCTGAGC
AGAAATCAACAGCAAGGACAAGCTGCATCTGTATCTGCATCTGATTCAGT
CTTTGAGCGCGCAAGCAATCCGGTCCAAGAGCTTTATACAAAAACTGAGA
GCCGGATCAGTCAAGGCATGATGAATATGAAGAGTGCTGGTGAACATTTC
AGATTTAAAAGCGCGGTAACAGATCAAATCGATGTATCCACAGCGGGAA
CGACGTACTGTCCTGATGTTGTTGGCCCTGTACAGCAGCAACAAACTTTCC
CACTACCATCATTTGGTTTTGATGGAGACTGCCAATCTCATCATCCAAGAA
ACAACTTAGCTTTCCCTGGTAATCTCGAAGCCGTAACTTCTGATCCACTCT
ATTCTCAAAAGGACTTTCAAAACTTGGTTCCCAACTATGGCAACACACCA
AGAGACATTGAGACGGAGCTGTCCAGTGCTGCAATCAGTTCTCAGTCATT
TGGTATTCCCAGCATTCCCTTTAAGCCCGGATGTTCAAATGAGGTTGGCGG
CATCAATGATTCAGGAATCATGAATGGTGGAGGACTGTGGCCCAATCAGA
CTCAACGAATGCGAACATATACAAAGGTTCAAAAACGAGGGTCAGTAGG
TAGATCAATAGATGTTACCCGTTATAGCGGCTATGATGAACTTAGGCATG
ACTTAGCGAGAATGTTTGGCATCGAAGGACAGCTCGAAGATCCGCTAACC
TCTGATTGGAAACTCGTCTACACCGATCACGAAAACGATATTTTACTAGTT
GGTGATGATCCTTGGGAAGAGTTTGTGAACTGCGTGCAGAACATAAAGAT
ACTATCATCAGTAGAAGTTCAGCAAATGAGCTTAGACGGAGATCTTGCAG
CTATCCCAACCACAAACCAAGCCTGCAGCGAAACAGACAGCGGAAATGC
TTGGAAAGTACACTATGAAGACACTTCTGCTGCAGCTTCTTTCAACAGAT
AG
SEQ ID No. 14
AtARF19 peptide sequence
MKAPSNGFLPSSNEGEKKPINSQLWHACAGPLVSLPPVGSLVVYFPQGHSEQ
VAASMQKQTDFIPNYPNLPSKLICLLHSVTLHADTETDEVYAQMTLQPVNKY
DREALLASDMGLKLNRQPTEFFCKTLTASDTSTHGGFSVPRRAAEKIFPPLDF
SMQPPAQEIVAKDLHDTTWTFRHIYRGQPKRHLLTTGWSVFVSTKRLFAGDS
VLFVRDEKSQLMLGIRRANRQTPTLSSSVISSDSMHIGILAAAAHANANSSPFT
IFFNPRASPSEFVVPLAKYNKALYAQVSLGMRFRMMFETEDCGVRRYMGTV
TGISDLDPVRWKGSQWRNLQVGWDESTAGDRPSRVSIWEIEPVITPFYICPPPF
FRPKYPRQPGMPDDELDMENAFKRAMPWMGEDFGMKDAQSSMFPGLSLVQ
WMSMQQNNPLSGSATPQLPSALSSFNLPNNFASNDPSKLLNFQSPNLSSANSQ
FNKPNTVNHISQQMQAQPAMVKSQQQQQQQQQQHQHQQQQLQQQQQLQM
SQQQVQQQGIYNNGTIAVANQVSCQSPNQPTGFSQSQLQQQSMLPTGAKMT
HQNINSMGNKGLSQMTSFAQEMQFQQQLEMHNSSQLLRNQQEQSSLHSLQQ
NLSQNPQQLQMQQQSSKPSPSQQLQLQLLQKLQQQQQQQSIPPVSSSLQPQLS
ALQQTQSHQLQQLLSSQNQQPLAHGNNSFPASTFMQPPQIQVSPQQQGQMSN
KNLVAAGRSHSGHTDGEAPSCSTSPSANNTGHDNVSPTNFLSRNQQQGQAAS
VSASDSVFERASNPVQELYTKTESRISQGMMNMKSAGEHFRFKSAVTDQIDV
STAGTTYCPDVVGPVQQQQTFPLPSFGFDGDCQSHHPRNNLAFPGNLEAVTS
DPLYSQKDFQNLVPNYGNTPRDIETELSSAAISSQSFGIPSIPFKPGCSNEVGGI
NDSGIMNGGGLWPNQTQRMRTYTKVQKRGSVGRSIDVTRYSGYDELRHDL
ARMFGIEGQLEDPLTSDWKLVYTDHENDILLVGDDPWEEFVNCVQNIKILSS
VEVQQMSLDGDLAAIPTTNQACSETDSGNA WKVHYEDTSA AASFNR
SEQ ID No. 15
AtARF7 nucleic acid sequence
TGAAAGCTCCTTCATCAAATGGAGTTTCTCCTAATCCTGTTGAAGGAGAA
AGGAGAAATATAAACTCAGAGCTATGGCACGCTTGTGCTGGGCCATTGAT
TTCGTTGCCTCCAGCAGGAAGTCTTGTTGTTTACTTCCCTCAAGGTCACAG
TGAGCAAGTCGCGGCTTCAATGCAGAAGCAGACTGATTTCATACCAAGTT
ACCCGAATCTTCCTTCCAAGCTCATATGCATGCTCCACAATGTTACACTGA
ATGCTGATCCTGAGACGGATGAGGTCTATGCGCAGATGACTCTTCAGCCA
GTAAACAAATATGACAGAGATGCATTGCTTGCTTCTGACATGGGTCTTAA
GCTAAACAGACAACCTAATGAATTTTTCTGCAAAACCCTCACGGCGAGTG
ACACAAGTACTCACGGTGGATTTTCTGTACCCCGACGAGCTGCTGAGAAA
ATCTTTCCTGCTCTGGATTTCTCGATGCAACCACCTTGTCAGGAGCTTGTT
GCTAAGGATATTCATGACAACACATGGACTTTCAGACATATTTATCGAGG
TCAACCAAAAAGGCACTTGCTAACTACAGGCTGGAGTGTGTTTGTCAGCA
CGAAAAGGCTCTTTGCTGGAGACTCTGTTCTTTTTATAAGAGATGGAAAG
GCGCAACTTCTGTTGGGGATAAGACGTGCAAATAGACAACAGCCTGCACT
TTCTTCATCTGTAATATCAAGTGATAGCATGCACATCGGAGTTCTTGCAGC
TGCAGCTCATGCTAATGCTAATAACAGTCCTTTCACCATTTTCTACAACCC
GAGGTGGGCTGCTCCTGCTGAGTTTGTGGTTCCTTTAGCCAAGTATACCAA
AGCGATGTACGCTCAAGTTTCCCTCGGTATGCGGTTTAGAATGATATTTGA
GACTGAAGAATGTGGAGTTCGTCGGTATATGGGTACAGTTACCGGTATCA
GTGATCTTGATCCAGTGAGATGGAAAAACTCTCAGTGGCGGAATCTTCAG
ATTGGATGGGATGAGTCAGCTGCTGGTGATAGGCCCAGTCGAGTTTCAGT
TTGGGACATTGAACCGGTTTTAACTCCTTTCTACATATGTCCTCCTCCATTT
TTCCGACCTCGCTTTTCTGGACAACCTGGAATGCCAGATGATGAGACTGA
CATGGAGTCTGCACTGAAGAGAGCAATGCCATGGCTTGATAATAGCTTAG
AGATGAAAGACCCTTCGAGTACTATCTTTCCTGGTCTGAGTTTAGTTCAGT
GGATGAATATGCAGCAGCAGAACGGCCAGCTACCCTCTGCTGCTGCACAG
CCAGGTTTCTTCCCATCAATGCTTTCGCCAACCGCGGCGCTGCACAACAAT
CTTGGCGGCACTGATGATCCCTCCAAGTTACTGAGCTTTCAGACGCCGCA
CGGGGGGATTTCCTCCTCAAATCTCCAATTTAACAAACAGAATCAGCAAG
CCCCAATGTCTCAGTTGCCTCAGCCACCAACTACGTTGTCCCAACAACAG
CAGCTGCAGCAATTGTTGCACTCCTCTTTGAACCATCAACAACAGCAATC
GCAGTCTCAACAACAGCAACAACAACAACAGTTGCTGCAGCAGCAACAA
CAATTGCAGTCTCAACAACACAGCAACAACAATCAATCGCAGTCTCAGCA
ACAACAACAATTGCTCCAGCAGCAACAACAACAACAACTGCAGCAACAA
CATCAACAACCGTTACAGCAACAGACTCAGCAGCAGCAGCTAAGAACAC
AGCCATTGCAATCTCACTCGCATCCACAGCCACAACAGTTACAACAACAT
AAGTTGCAGCAACTTCAGGTTCCACAGAATCAGCTTTACAATGGTCAACA
AGCAGCGCAGCAGCATCAGTCGCAACAAGCATCTACACATCATTTGCAAC
CACAATTAGTTTCGGGATCAATGGCAAGCAGTGTCATCACGCCTCCGTCC
AGCTCCCTTAATCAAAGCTTTCAACAGCAACAACAACAGTCTAAGCAACT
TCAACAAGCACATCACCATTTAGGTGCTAGCACTAGCCAGAGTAGTGTAA
TTGAAACCAGCAAGTCTTCATCCAATCTGATGTCCGCACCGCCGCAAGAG
ACACAGTTTTCACGACAAGTAGAACAGCAGCAGCCTCCTGGTCTCAACGG
GCAGAATCAGCAAACACTTTTGCAGCAGAAAGCTCACCAGGCACAGGCC
CAACAGATATTCCAGCAGAGTCTCTTGGAACAGCCGCATATACAGTTTCA
GCTGTTACAGAGATTACAACAGCAACAGCAGCAGCAATTTCTTTCGCCGC
AGTCTCAGTTACCACACCATCAATTGCAAAGCCAGCAGTTGCAACAGCTG
CCTACTCTCTCTCAAGGTCATCAGTTTCCGTCATCTTGCACTAACAATGGC
TTATCGACGTTGCAACCACCTCAAATGCTGGTGAGCCGACCTCAGGAAAA
ACAAAACCCACCGGTTGGGGGAGGGGTCAAAGCTTATTCAGGCATCACA
GATGGAGGAGATGCACCTTCCTCTTCAACGTCGCCTTCCACCAACAACTG
TCAGATCTCTTCTTCAGGCTTTCTCAACAGAAGCCAAAGCGGGCCAGCGA
TCTTGATACCTGATGCAGCGATTGATATGTCTGGTAATCTTGTTCAGGATC
TTTACAGCAAATCCGATATGCGGCTAAAACAAGAACTCGTGGGTCAGCAA
AAGTCCAAAGCTAGTTTAACAGATCATCAACTAGAAGCATCTGCCTCTGG
AACTTCTTACGGTTTAGATGGAGGCGAAAACAACAGACAACAAAATTTCT
TGGCTCCAACTTTTGGCCTTGACGGTGATTCCAGGAACAGCTTGCTCGGTG
GAGCTAATGTTGATAATGGCTTTGTGCCTGACACGCTACTCTCGAGGGGA
TATGACTCCCAGAAAGATCTTCAGAACATGCTTTCAAACTATGGAGGAGT
GACAAATGACATTGGTACAGAGATGTCTACTTCAGCTGTAAGAACTCAAT
CTTTTGGTGTCCCCAATGTGCCCGCCATTTCGAACGATCTAGCTGTCAACG
ATGCTGGAGTTCTTGGTGGTGGATTGTGGCCAGCTCAGACTCAGCGAATG
CGAACTTATACAAAGGTGCAAAAACGAGGCTCAGTGGGGAGATCAATAG
ACGTCAACCGTTACAGAGGTTACGATGAGCTGAGGCATGATCTAGCGCGC
ATGTTTGGGATCGAAGGACAGCTCGAAGATCCTCAAACATCTGACTGGAA
ACTTGTTTATGTCGATCATGAAAATGACATCCTCCTCGTCGGCGATGATCC
ATGGGAAGAATTCGTAAACTGTGTTCAGAGCATTAAGATCCTTTCATCAG
CTGAGGTTCAGCAGATGAGCTTAGACGGGAACTTTGCCGGTGTACCAGTT
ACTAATCAAGCTTGTAGTGGCGGTGACAGTGGCAATGCTTGGAGAGGTCA
TTATGATGATAACTCAGCCACTTCGTTTAACCGGTGA
SEQ ID No. 16
AtARF7 peptide sequence
MKAPSSNGVSPNPVEGERRNINSELWHACAGPLISLPPAGSLVVYFPQGHSEQ
VAASMQKQTDFIPSYPNLPSKLICMLHNVTLNADPETDEVYAQMTLQPVNK
YDRDALLASDMGLKLNRQPNEFFCKTLTASDTSTHGGFSVPRRAAEKIFPAL
DFSMQPPCQELVAKDIHDNTWTFRHIYRGQPKRHLLTTGWSVFVSTKRLFAG
DSVLFIRDGKAQLLLGIRRANRQQPALSSSVISSDSMHIGVLAAAAHANANNS
PFTIFYNPRAAPAEFVVPLAKYTKAMYAQVSLGMRFRMIFETEECGVRRYMG
TVTGISDLDPVRWKNSQWRNLQIGWDESAAGDRPSRVSVWDIEPVLTPFYIC
PPPFFRPRFSGQPGMPDDETDMESALKRAMPWLDNSLEMKDPSSTIFPGLSLV
QWMNMQQQNGQLPSAAAQPGFFPSMLSPTAALHNNLGGTDDPSKLLSFQTP
HGGISSSNLQFNKQNQQAPMSQLPQPPTTLSQQQQLQQLLHSSLNHQQQQSQ
SQQQQQQQQLLQQQQQLQSQQHSNNNQSQSQQQQQLLQQQQQQQLQQQH
QQPLQQQTQQQQLRTQPLQSHSHPQPQQLQQHKLQQLQVPQNQLYNGQQA
AQQHQSQQASTHHLQPQLVSGSMASSVITPPSSSLNQSFQQQQQQSKQLQQA
HHHLGASTSQSSVIETSKSSSNLMSAPPQETQFSRQVEQQQPPGLNGQNQQTL
LQQKAHQAQAQQIFQQSLLEQPHIQFQLLQRLQQQQQQQFLSPQSQLPHHQL
QSQQLQQLPTLSQGHQFPSSCTNNGLSTLQPPQMLVSRPQEKQNPPVGGGVK
AYSGITDGGDAPSSSTSPSTNNCQISSSGFLNRSQSGPAILIPDAAIDMSGNLVQ
DLYSKSDMRLKQELVGQQKSKASLTDHQLEASASGTSYGLDGGENNRQQNF
LAPTFGLDGDSRNSLLGGANVDNGFVPDTLLSRGYDSQKDLQNMLSNYGGV
TNDIGTEMSTSAVRTQSFGVPNVPAISNDLAVNDAGVLGGGLWPAQTQRMR
TYTKVQKRGSVGRSIDVNRYRGYDELRHDLARMFGIEGQLEDPQTSDWKLV
YVDHENDILLVGDDPWEEFVNCVQSIKILSSA EVQQMSLDGN
FAGVPVTNQACSGGDSGNAW RGHYDDNSATSFNR
SEQ ID No. 17
OsARF7 nucleic acid sequence
ATGAAGGATCAGGGATCATCCGGTGTGTCTCCCGCCCCAGGGGAAGGGG
AGAAGAAAGCCATCAATTCGGAGCTATGGCATGCTTGTGCCGGGCCTCTT
GTGTCGCTGCCGCCGGTGGGCAGTCTCGTCGTGTACTTCCCTCAGGGTCAT
AGCGAGCAGGTTGCTGCTTCCATGCACAAGGAGCTGGACAACATCCCTGG
TTATCCCTCTCTTCCGTCTAAGCTGATCTGCAAACTTCTGAGTCTCACCTTA
CATGCAGATTCTGAAACTGATGAAGTTTATGCTCAGATGACACTTCAACC
AGTCAATAAATATGATCGAGATGCAATGCTGGCATCTGAACTGGGCCTGA
AGCAAAACAAGCAACCAGCGGAGTTCTTTTGCAAAACGCTGACGGCGAG
CGACACAAGTACCCATGGTGGATTTTCAGTGCCACGTCGTGCGGCGGAGA
AGATATTTCCACCACTAGACTTTACCATGCAACCACCAGCACAAGAGCTC
ATCGCCAAGGATCTGCATGATATTTCATGGAAATTTCGACACATTTACCG
AGGTCAACCAAAGAGGCACCTTCTGACAACTGGTTGGAGCGTCTTTGTCA
GCACAAAAAGGCTTCTAGCTGGTGATTCAGTTCTGTTTATAAGGGATGAG
AAATCTCAGCTTCTATTAGGCATACGTCGTGCTACCAGACCCCAACCAGC
TCTATCGTCATCAGTTCTATCAAGTGATAGCATGCACATTGGGATTCTAGC
TGCTGCAGCACATGCTGCTGCAAACAGTAGCCCATTTACTATTTTCTACAA
TCCAAGGGCAAGTCCATCAGAATTTGTCATTCCTTTAGCGAAATATAACA
AGGCTTTGTATACACAAGTATCTCTTGGAATGCGGTTCAGAATGCTGTTTG
AGACAGAGGATTCAGGGGTTCGAAGATATATGGGAACAATCACAGGTAT
TGGTGACTTGGATCCAGTGCGCTGGAAGAACTCTCATTGGCGAAACCTTC
AGGTTGGTTGGGATGAATCAACAGCATCTGAGAGGCGCACTCGTGTTTCA
ATATGGGAGATTGAACCAGTCGCGACACCTTTTTATATTTGTCCACCACCA
TTTTTCAGGCCAAAACTTCCTAAGCAGCCAGGAATGCCAGATGATGAAAA
TGAAGTTGAGAGTGCTTTCAAAAGAGCCATGCCATGGCTTGCTGATGACT
TTGCCCTGAAAGATGTGCAAAGTGCATTATTTCCAGGTCTGAGCCTAGTCC
AATGGATGGCTATGCAACAGAATCCTCAGATGCTAACAGCTGCGTCCCAA
ACAGTGCAATCACCGTACTTGAACTCCAATGCATTGGCTATGCAGGATGT
GATGGGTAGTAGCAACGAGGACCCAACAAAAAGATTGAACACACAGGCA
CAAAATATGGTTTTACCTAATTTACAGGTTGGCTCAAAAGTGGATCACCCT
GTAATGTCTCAACATCAACAGCAGCCACACCAACTATCACAACAGCAGCA
GGTCCAGCCATCGCAGCAAAGTTCTGTGGTTTTACAGCAACATCAAGCCC
AGTTGCTGCAGCAGAACGCCATTCACTTGCAGCAGCAGCAAGAACATCTC
CAGCGGCAGCAGTCACAACCGGCACAGCAGTTGAAGGCTGCTTCAAGTCT
GCATTCAGTGGAACAGCACAAGCTGAAAGAACAGACTTCAGGTGGGCAG
GTTGCCTCACAAGCACAAATGTTAAACCAGATTTTCCCACCATCTTCATCG
CAGCTACAACAGTTAGGTTTACCCAAGTCACCTACTCATCGCCAAGGGTT
GACAGGATTACCAATTGCAGGTTCTTTGCAGCAGCCCACACTAACTCAGA
CATCTCAAGTCCAGCAAGCAGCCGAATATCAGCAGGCCCTCCTACAGAGT
CAGCAACAGCAACAGCAACTGCAACTGCAACAACTATCACAACCAGAAG
TACAGCTGCAGCTGCTTCAAAAGATTCAACAACAAAACATGCTATCTCAG
CTGAACCCACAACATCAGTCCCAGTTGATTCAACAATTGTCTCAGAAAAG
CCAGGAAATTCTACAGCAACAAATTTTGCAACATCAATTTGGTGGGTCTG
ATTCTATTGGTCAACTCAAGCAATCACCATCGCAGCAAGCTCCTTTAAAC
CACATGACAGGATCTTTGACGCCCCAGCAACTTGTCAGATCACATTCGGC
ACTTGCTGAGAGTGGGGATCCATCCAGTTCAACTGCTCCATCCACCAGCC
GTATTTCTCCAATAAATTCGCTGAGTAGGGCAAACCAAGGAAGCAGAAAT
TTAACTGACATGGTGGCAACACCACAAATTGACAACTTACTTCAGGAAAT
TCAAAGCAAGCCAGATAATCGAATTAAGAATGACATACAGAGCAAAGAA
ACAGTCCCTATACATAACCGACATCCAGTTTCTGATCAACTTGATGCATCA
TCTGCTACCTCCTTTTGTTTAGACGAGAGCCCACGAGAAGGTTTTTCCTTC
CCTCCAGTTTGTTTGGATAACAATGTTCAAGTTGATCCAAGAGATAACTTT
CTTATTGCGGAAAATGTGGACGCATTGATGCCAGATGCCCTGTTGTCAAG
AGGTATGGCTTCAGGAAAGGGCATGTGCACTCTGACTTCTGGACAAAGGG
ATCACAGGGATGTCGAGAATGAGCTATCATCTGCTGCATTCAGTTCCCAG
TCATTTGGTGTGCCTGACATGTCCTTTAAGCCTGGATGTTCAAGTGACGTT
GCTGTTACTGATGCCGGAATGCCAAGCCAAGGTTTGTGGAATAATCAAAC
ACAACGGATGAGAACTTTCACTAAGGTTCAAAAGCGTGGTTCTGTGGGGA
GATCAATTGATATCACAAGATATCGAGATTATGATGAACTTAGGCATGAT
CTTGCATGCATGTTTGGTATCCAAGGTCAACTTGAAGATCCATATAGGAT
GGATTGGAAGCTAGTCTATGTTGATCATGAGAATGATATCCTTCTTGTCGG
CGACGACCCTTGGGAGGAATTTGTGGGCTGTGTGAAGAGCATCAAAATAC
TCTCAGCTGCTGAAGTACAACAGATGAGCTTGGATGGTGACCTTGGTGGC
GTCCCTCCACAAACACAGGCCTGTAGTGCCTCTGATGATGCAAATGCATG
GAGAGGTTGA
SEQ ID No. 18
OsARF7 peptide sequence
MKDQGSSGVSPAPGEGEKKAINSELWHACAGPLVSLPPVGSLVVYFPQGHSE
QVAASMHKELDNIPGYPSLPSKLICKLLSLTLHADSETDEVYAQMTLQPVNK
YDRDAMLASELGLKQNKQPAEFFCKTLTASDTSTHGGFSVPRRAAEKIFPPL
DFTMQPPAQELIAKDLHDISWKFRHIYRGQPKRHLLTTGWSVFVSTKRLLAG
DSVLFIRDESQLLLGIRRATRPQPALSSSVLSSDSMHIGILAAAAHAAANSSPF
TIFYNPRASPSEFVIPLAKYNKALYTQVSLGMRFRMLFETEDSGVRRYMGTIT
GIGDLDPVRWKNSHWRNLQVGWDESTASERRTRVSIWEIEPVATPFYICPPPF
FRPKLPKQPGMPDDENEVESAFKRAMPWLADDFALKDVQSALFPGLSLVQW
MAMQQNPQMLTAASQTVQSPYLNSNALAMQDVMGSNEDPTKRLNTQAQN
MVLPNLQVGSKVDHPVMSQHQQQPHQLSQQQQVQPSQQSSVVLQQHQAQL
LQQNAIHLQQQQEHLQRQQSQPAQQLKAASSLHSVEQHKLKEQTSGGQVAS
QAQMLNQIFPPSSSQLQQLGLPKSPTHRQGLTGLPIAGSLQQPTLTQTSQVQQ
AAEYQQALLQSQQQQQQLQLQQLSQPEVQLQLLQKIQQQNMLSQLNPQHQS
QLIQQLSQKSQEILQQQILQHQFGGSDSIGQLKQSPSQQAPLNHMTGSLTPQQ
LVRSHSALAESGDPSSSTAPSTSRISPINSLSRANQGSRNLTDMVATPQIDNLL
QEIQSKPDNRIKNDIQSKETVPIHNRHPVSDQLDASSATSFCLDESPREGFSFPP
VCLDNNVQVDPRDNFLIAENVDALMPDALLSRGMASGKGMCTLTSGQRDH
RDVENELSSAAFSSQSFGVPDMSFKPGCSSDVAVTDAGMPSQGLWNNQTQR
MRTFTKVQKRGSVGRSIDITRYRDYDELRHDLACMFGIQGQLEDPYRMDWK
LVYVDHENDILLVGDDPWEEFVGCVKSIKILSAAEVQQMSLDGDLGGVPPQT
QACSASDDANAWRG
SEQ ID No. 19
OsARF19 nucleic acid sequence
ATGATGAAGCAGGCGCAGCAGCAGCCGCCGCCGCCACCGGCGAGCTCTG
CGGCGACGACGACCACCGCGATGGCAGCCGCTGCGGCGGCGGCGGTGGT
GGGGAGCGGGTGCGAAGGGGAGAAGACGAAGGCGCCGGCGATCAACTC
GGAGCTGTGGCACGCCTGCGCGGGGCCGCTGGTGTCGCTGCCGCCGGCGG
GCAGCCTCGTCGTCTACTTCCCCCAGGGCCACAGCGAGCAGGCGGACCCA
GAAACAGATGAAGTGTATGCACAAATGACTCTTCAGCCAGTTACTTCATA
TGGGAAGGAGGCCCTGCAGTTATCAGAGCTTGCACTCAAACAAGCGAGA
CCACAGACAGAATTCTTTTGCAAGACACTGACTGCAAGTGATACAAGTAC
TCATGGAGGCTTCTCTGTGCCTCGTCGAGCTGCAGAAAAGATATTTCCTCC
ACTGGACTTCTCAATGCAACCACCTGCACAAGAACTACAGGCCAGGGATT
TGCATGATAATGTGTGGACATTCCGTCACATATATCGGGGTCAGCCAAAA
AGGCATCTGCTTACCACTGGCTGGAGTCTATTTGTAAGCGGCAAGAGGTT
ATTTGCTGGAGATTCTGTCATTTTTGTCAGGGATGAAAAGCAGCAACTTCT
ATTAGGAATCAGGCGTGCTAACCGACAGCCAACTAACATATCATCATCTG
TCCTTTCAAGTGACAGCATGCACATAGGGATTCTTGCTGCTGCAGCCCATG
CTGCTGCCAACAATAGCCCATTTACCATCTTTTATAACCCTAGGGCCAGTC
CTACTGAATTTGTTATCCCATTTGCTAAGTATCAGAAGGCAGTCTATGGTA
ATCAAATATCTTTAGGGATGCGCTTTCGCATGATGTTTGAGACTGAGGAA
TTAGGAACACGAAGATACATGGGAACAATAACTGGCATAAGTGATCTAG
ATCCAGTAAGATGGAAAAACTCGCAGTGGCGCAACTTACAGGTTGGTTGG
GATGAATCCGCAGCCGGTGAAAGGCGAAATAGGGTTTCTATCTGGGAGAT
TGAACCGGTCGCTGCTCCATTTTTCATATGTCCTCCACCATTTTTTGGTGCG
AAGCGGCCCAGGCAATTAGATGACGAGTCCTCGGAAATGGAGAATCTCTT
AAAGAGGGCTATGCCTTGGCTTGGTGAGGAAATATGCATAAAGGATCCTC
AGACTCAGAACACCATAATGCCTGGGCTGAGCTTGGTTCAGTGGATGAAC
ATGAACATGCAACAGAGCTCCTCATTTGCGAATACAGCCATGCAGTCTGA
GTACCTTCGATCATTGAGCAACCCCAACATGCAAAATCTTGGTGCCGCCG
ATCTCTCTAGGCAATTATGCCTGCAGAACCAGCTTCTTCAACAGAACAAT
ATACAGTTTAATACTCCCAAACTTTCTCAGCAAATGCAGCCAGTCAATGA
GTTAGCAAAGGCAGGCATTCCGTTGAATCAGCTTGGTGTGAGCACCAAAC
CTCAGGAACAGATTCATGATGCTAGCAACCTTCAGAGGCAACAACCTTCC
ATGAACCATATGCTTCCTTTGAGCCAAGCTCAAACCAATCTTGGCCAAGC
TCAGGTCCTTGTCCAAAATCAAATGCAACAGCAACATGCATCTTCAACTC
AAGGTCAACAACCAGCTACCAGCCAGCCCTTGCTTCTGCCCCAGCAGCAG
CAACAGCAGCAGCAGCAGCAGCAACAACAACAACAACAGCAACAACAAC
AAAAATTGCTACAACAGCAGCAGCAACAGCTTTTGCTCCAGCAACAGCAG
CAATTGAGTAAGATGCCTGCACAGTTGTCAAGTCTGGCGAATCAGCAGTT
TCAGCTAACTGATCAACAGCTTCAGCTGCAACTGTTACAAAAACTACAGC
AACAACAGCAGTCATTGCTTTCACAACCTGCAGTCACCCTTGCACAATTA
CCTCTGATCCAAGAACAGCAGAAGTTACTTCTGGATATGCAACAGCAGCT
GTCAAACTCCCAAACACTTTCCCAACAACAAATGATGCCTCAACAAAGTA
CCAAGGTTCCATCACAGAACACACCATTGCCACTGCCTGTGCAACAAGAG
CCACAACAGAAGCTTCTACAGAAGCAAGCGATGCTAGCAGACACTTCAG
AAGCTGCCGTTCCGCCGACCACATCAGTCAATGTCATTTCAACAACTGGA
AGCCCTTTGATGACAACTGGTGCTACTCATTCTGTACTTACAGAAGAAATC
CCTTCTTGTTCAACATCACCATCCACAGCTAATGGCAATCACCTTCTACAA
CCAATACTTGGTAGGAACAAACATTGTAGCATGATCAACACAGAAAAGGT
TCCTCAGTCTGCTGCTCCTATGTCAGTTCCAAGCTCCCTTGAAGCTGTCAC
AGCAACCCCGAGAATGATGAAGGATTCACCAAAGTTGAACCATAATGTTA
AACAAAGTGTAGTGGCTTCAAAATTAGCAAATGCTGGGACTGGTTCTCAA
AATTATGTGAACAATCCACCTCCAACGGACTATCTGGAAACTGCTTCTTCC
GCAACTTCAGTGTGGCTTTCCCAGAATGATGGACTTCTACATCAAAATTTC
CCTATGTCCAACTTCAACCAGCCACAGATGTTCAAAGATGCTCCTCCTGAT
GCTGAAATTCATGCTGCTAATACAAGTAACAATGCATTGTTTGGAATCAA
TGGTGATGGTCCGCTGGGCTTCCCTATAGGACTAGGAACAGATGATTTCC
TGTCGAATGGAATTGATGCTGCCAAGTACGAGAACCATATCTCAACAGAA
ATTGATAATAGCTACAGAATTCCGAAGGATGCCCAGCAAGAAATATCATC
CTCAATGGTTTCACAGTCATTTGGTGCATCAGATATGGCATTTAATTCAAT
TGATTCCACGATCAACGATGGTGGCTTTTTGAACCGGAGTTCTTGGCCTCC
TGCCGCTCCCTTAAAGAGGATGAGGACATTCACCAAGGTATATAAGCGAG
GAGCTGTAGGCCGGTCCATTGACATGAGTCAGTTCTCTGGATATGATGAA
TTAAAGCATGCTCTGGCACGGATGTTCAGTATAGAGGGGCAACTTGAGGA
ACGGCAGAGAATTGGTTGGAAGCTCGTTTACAAGGATCATGAAGATGACA
TCCTACTTCTTGGCGACGACCCATGGGAGGAATTTGTCGGTTGCGTGAAA
TGCATTAGGATCCTTTCACCTCAAGAAGTTCAGCAGATGAGCTTGGAGGG
TTGTGATCTCGGGAACAACATTCCCCCGAATCAGGCCTGCAGCAGCTCAG
ACGGAGGGAATGCATGGAGGGCTCGCTGCGATCAGAACTCCGAGGCCAT
TCTTAAGATCTCCATGATGAAATCAAAAGTTGAAGATGTCAGGTATTGGA
ATACTGCGTAA
SEQ ID No. 20
OsARF19 peptide sequence
MMKQAQQQPPPPPASSAATTTTAMAAAAAAAVVGSGCEGEKTKAPAINSEL
WHACAGPLVSLPPAGSLVVYFPQGHSEQADPETDEVYAQMTLQPVTSYGKE
ALQLSELALKQARPQTEFFCKTLTASDTSTHGGFSVPRRAAEKIFPPLDFSMQ
PPAQELQARDLHDNVWTFRHIYRGQPKRHLLTTGWSLFVSGKRLFAGDSVIF
VRDEKQQLLLGIRRANRQPTNISSSVLSSDSMHIGILAAAAHAAANNSPFTIFY
NPRASPTEFVIPFAKYQKAVYGNQISLGMRFRMMFETEELGTRRYMGTITGIS
DLDPVRWKNSQWRNLQVGWDESAAGERRNRVSIWEIEPVAAPFFICPPPFFG
AKRPRQLDDESSEMENLLKRAMPWLGEEICIKDPQTQNTIMPGLSLVQWMN
MNMQQSSSFANTAMQSEYLRSLSNPNMQNLGAADLSRQLCLQNQLLQQNNI
QFNTPKLSQQMQPVNELAKAGIPLNQLGVSTKPQEQIHDASNLQRQQPSMNH
MLPLSQAQTNLGQAQVLVQNQMQQQHASSTQGQQPATSQPLLLPQQQQQQ
QQQQQQQQQQQQQQKLLQQQQQQLLLQQQQQLSKMPAQLSSLANQQFQLT
DQQLQLQLLQKLQQQQQSLLSQPAVTLAQLPLIQEQQKLLLDMQQQLSNSQT
LSQQQMMPQQSTKVPSQNTPLPLPVQQEPQQKLLQKQAMLADTSEAAVPPT
TSVNVISTTGSPLMTTGATHSVLTEEIPSCSTSPSTANGNHLLQPILGRNKHCS
MINTEKVPQSAAPMSVPSSLEAVTATPRMMKDSPKLNHNVKQSVVASKLAN
AGTGSQNYVNNPPPTDYLETASSATSVWLSQNDGLLHQNFPMSNFNQPQMF
KDAPPDAEIHAANTSNNALFGINGDGPLGFPIGLGTDDFLSNGIDAAKYENHI
STEIDNSYRIPKDAQQEISSSMVSQSFGASDMAFNSIDSTINDGGFLNRSSWPP
AAPLKRMRTFTKVYKRGAVGRSIDMSQFSGYDELKHALARMFSIEGQLEER
QRIGWKLVYKDHEDDILLLGDDPWEEFVGCVKCIRILSPQEVQQMSLEGCDL
GNNIPPNQACSSSDGGNAWRARCDQNSEAILKISMMKSKVEDVRYWNTA

The invention is further described by the following numbered paragraphs:

1. A method for modifying growth of a plant comprising altering the SUMOylation status of a target protein or altering the interaction of a SUMOylated target protein with its receptor.

2. A method for modifying growth of a plant according to paragraph 1 comprising altering the SUMOylation status of a target protein.

3. The method of paragraph 1 or 2 wherein growth is increased.

4. A method according to a preceding paragraph wherein growth is increased under stress conditions.

5. The method of according to a preceding paragraph wherein SUMOylation of the target protein is decreased or prevented said method comprising expressing a nucleic acid sequence encoding a mutant target protein in a plant wherein said nucleic acid sequence has been altered to decrease or prevent SUMOylation of said target protein.

6. The method according to paragraph 5 wherein said method comprises altering a codon encoding a conserved lysine (K) residue in said nucleic acid sequence.

7. A method according to paragraph 4 or paragraph 5 for increasing growth of a plant under stress conditions comprising expressing a gene construct comprising a nucleic acid that encodes a RGL-1, RGL-2, GAI, RGL-3 polypeptide as defined in SEQ ID No. 2, 6, 8 or 12 or a homologue or orthologue thereof but which comprises a substitution of one or more conserved residue in the SUMOylation site in a plant.

8. A method according to paragraph 7 wherein said stress is drought or salinity.

9. The method according to paragraph 1 to 4 comprising altering binding of the SUMOylated target protein to its receptor.

10. The method according to paragraph 9 comprising expressing a nucleic acid sequence encoding a mutant receptor protein wherein the SIM site in said nucleic acid sequence has been altered to decrease or prevent binding of the SUMOylated target protein.

11. A method for according to paragraph 10 for increasing growth of a plant under stress conditions, comprising expressing a gene construct encoding a mutant GID1 receptor in a plant wherein the mutation in said receptor prevents binding of a SUMOylated DELLA polypeptide selected from RGL-1, RGL-2, GAI, RGL-3 as defined in SEQ ID No. 2, 6, 8 or 12 or a homologue or orthologue thereof to its receptor.

12. A method according to paragraph 11 wherein the mutant GID receptor is selected from SEQ ID No. 10, a homologue or orthologue thereof but comprises a mutation in the SIM site.

13. A method according to any of paragraphs 11 to 12 wherein the mutation is a substitution of W or V.

14. A method according to any of paragraphs 11 to 13 wherein said stress is drought or salinity.

15. A transgenic plant obtained or obtainable by one of the methods of paragraphs 1 to 14.

16. A transgenic plant expressing a gene encoding for a mutant target protein involved in growth regulation wherein said protein comprises an altered SUMOylation site or expressing a gene encoding for a mutant recptor protein comprising altered SIM site and wherein the unmodified receptor protein binds a target protein involved in growth regulation.

17. An isolated nucleic acid encoding for a RGL-1, RGL-2, GAI, RGL-3 polypeptide, homologue or orthologue thereof as defined in SEQ ID No. 2, 6, 8 or 12 but which comprises a substitution of one or more residue, for example K, in the conserved SUMOylation site.

18. A vector comprising an isolated nucleic acid according to paragraph 17.

19. A host cell comprising a vector according to paragraph 18.

20. A host cell according to paragraph 19 wherein said host cell is a plant or bacterial cell.

21. A transgenic plant expressing a nucleic acid construct comprising a nucleic acid as defined in paragraph 17 or a vector as defined in paragraph 18.

22. An isolated nucleic acid encoding for a GID1 a polypeptide as defined in SEQ ID No. 10, a homolog or ortholog thereof but which comprises a substitution of one or more conserved residue in the conserved SUMOylation site.

23. A vector comprising an isolated nucleic acid according to paragraph 22.

24. A host cell comprising a vector according to paragraph 23.

25. A host cell according to paragraph 34 wherein said host cell is a plant or bacterial cell.

26. A transgenic plant expressing a nucleic acid construct comprising a nucleic acid as defined in paragraph 22 or a vector as defined in paragraph 23.

27. A method for for producing a transgenic plant with improved yield and/or growth under stress conditions said method comprising
a) introducing into said plant and expressing a nucleic acid encoding an altered DELLA protein selected from GAI, RGL-1, 2 or 3 or their homologs or orthologues wherein the SUMOylation site is altered as described above or introducing into said plant and expressing a construct comprising a nucleic acid that encodes a GID1 a receptor as defined in SEQ ID No. 10 but which comprises a substitution of one or more residue within the SIM site, for example of the conserved W or V residue or the K residue in the conserved SUMOylation site and
b) obtaining a progeny plant derived from the plant or plant cell of step a).

28. A method for increasing stress tolerance comprising altering the SUMOylation status of a target protein or altering the interaction of a SUMOylated target protein with its receptor.

29. A method for altering root architecture, comprising preventing, decreasing or increasing SUMOylation of a AtARF19 or AtARF7 polypeptide as defined in SEQ ID No. 14 or 16 or a functional variant, homologue or orthologue thereof.

30. A method increasing the formation of lateral root in a plant by preventing or decreasing SUMOylation of a AtARF19 or AtARF7 polypeptide comprising expressing a nucleic acid construct comprising a nucleic acid that encodes for a AtARF19 or AtARF7 polypeptide as defined in SEQ ID No. 14 or 16 or a functional variant, homologue or orthologue thereof but which comprises a substitution of one or more conserved residue in the conserved SUMOylation site in a plant.

31. A method increasing the formation of a tap root system in a plant by increasing SUMOylation of a AtARF19 or AtARF7 polypeptide comprising expressing a nucleic acid construct comprising a nucleic acid that encodes for a AtARF19 or AtARF7 polypeptide as defined in SEQ ID No. 14 or 16 or a functional variant, homologue or orthologue thereof but which comprises additional SUMOylation sites in a plant.

32. A method for producing a plant with altered root architecture, comprising preventing, decreasing or increasing SUMOylation of a AtARF19 or AtARF7 polypeptide as defined in SEQ ID No. 14 or 16 or a functional variant, homologue or orthologue thereof.

33. A method according to paragraph 32 comprising expressing a nucleic acid construct comprising a nucleic acid that encodes for a AtARF19 or AtARF7 polypeptide as defined in SEQ ID No. 14 or 16 or a functional variant, homologue or orthologue thereof but which comprises a substitution of one or more residue, for example K, in the conserved SUMOylation site in a plant.

34. A method for increasing plant tolerance to nutrient deficient conditions, comprising preventing or decreasing SUMOylation of a AtARF19 or AtARF7 polypeptide as defined in SEQ ID No. 14 or 16 or a functional variant, homologue or orthologue thereof.

35. A method according to paragraph 34 comprising expressing a nucleic acid construct comprising a nucleic acid that encodes for a AtARF19 or AtARF7 polypeptide as defined in SEQ ID No. 14 or 16 or a functional variant, homologue or orthologue thereof but which comprises a substitution of one or more conserved residue in the conserved SUMOylation site in a plant.

36. An isolated nucleic acid encoding for a AtARF19 or AtARF7 polypeptide as defined in SEQ ID No. 14 or 16 or a functional variant, homologue or orthologue thereof but which comprises a substitution of one or more conserved residue in the conserved SUMOylation site.

37. An isolated nucleic acid encoding for a AtARF19 or AtARF7 polypeptide as defined in SEQ ID No. 14 or 16 or a functional variant, homologue or orthologue thereof but which comprises additional SUMOylation sites.

38. A vector comprising an isolated nucleic acid according to paragraph 36 or 37.

39. A host cell comprising a vector according to paragraph 38.

40. A host cell according to paragraph 39 wherein said host cell is a plant or bacterial cell.

41. A transgenic plant expressing a nucleic acid construct comprising a nucleic acid as defined in paragraph 36 or 37 or a vector as defined in paragraph 38.

42. A transgenic plant according to paragraph 41 wherein said plant has altered root architecture.

43. The use of a nucleic acid construct comprising a nucleic acid as defined in paragraph 36 or 37 or a vector as defined in paragraph 38 in altering root architecture.

44. An in vitro assay for identifying a target compound that increases SUMOylation.

45. A method for identifying a compound that regulates SUMOylation.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A method for modifying growth of a plant comprising altering the SUMOylation status of a target protein or altering the interaction of a SUMOylated target protein with its receptor.

2. A method for modifying growth of a plant according to claim 1
comprising altering the SUMOylation status of a target protein or
wherein growth is increased or

[symbol]

3. A method according to claim 1 wherein growth is increased under stress conditions.

4. The method according to claim 1 wherein SUMOylation of the target protein is decreased or prevented said method comprising expressing a nucleic acid sequence encoding a mutant target protein in a plant wherein said nucleic acid sequence has been altered to decrease or prevent SUMOylation of said target protein.

5. The method according to claim 4 wherein said method comprises altering a codon encoding a conserved lysine (K) residue in said nucleic acid sequence.

6. A method according to claim 3 for increasing growth of a plant under stress conditions comprising expressing a gene construct comprising a nucleic acid that encodes a RGL-1, RGL-2, GAI, RGL-3 polypeptide as defined in SEQ ID No. 2, 6, 8 or 12 or a homologue or orthologue thereof but which comprises a substitution of one or more conserved residue in the SUMOylation site in a plant.

7. A method according to claim 6 wherein said stress is drought or salinity.

8. The method according to claim 1 comprising altering binding of the SUMOylated target protein to its receptor.

9. The method according to claim 8 comprising expressing a nucleic acid sequence encoding a mutant receptor protein wherein the SIM site in said nucleic acid sequence has been altered to decrease or prevent binding of the SUMOylated target protein.

10. A method for according to claim 9 for increasing growth of a plant under stress conditions, comprising expressing a gene construct encoding a mutant GID1 receptor in a plant wherein the mutation in said receptor prevents binding of a SUMOylated DELLA polypeptide selected from RGL-1, RGL-2, GAI, RGL-3 as defined in SEQ ID No. 2, 6, 8 or 12 or a homologue or orthologue thereof to its receptor.

11. A method according to claim 10
wherein the mutant GID receptor is selected from SEQ ID No. 10, a homologue or orthologue thereof but comprises a mutation in the SIM site or
wherein the mutation is a substitution of W or V or
wherein said stress is drought or salinity.

12. An isolated nucleic acid encoding for
a RGL-1, RGL-2, GAI, RGL-3 polypeptide, homologue or orthologue thereof as defined in SEQ ID No. 2, 6, 8 or 12 but which comprises a substitution of one or more residue, for example K, in the conserved SUMOylation site or
a GID1a polypeptide as defined in SEQ ID No. 10, a homolog or ortholog thereof but which comprises a substitution of one or more conserved residue in the conserved SUMOylation site or
a AtARF19 or AtARF7 polypeptide as defined in SEQ ID No. 14 or 16 or a functional variant, homologue or orthologue thereof but which comprises a substitution of one or more conserved residue in the conserved SUMOylation site or
AtARF19 or AtARF7 polypeptide as defined in SEQ ID No. 14 or 16 or a functional variant, homologue or orthologue thereof but which comprises additional SUMOylation sites.

13. A vector comprising an isolated nucleic acid according to claim 12.

14. A host cell comprising a vector according to claim 13.

15. A host cell according to claim 14 wherein said host cell is a plant or bacterial cell.

16. A transgenic plant expressing a nucleic acid construct comprising a nucleic acid as defined in claim 12.

17. A transgenic plant according to claim 16 wherein said plant has altered root architecture.

18. A method for for producing a transgenic plant with improved yield and/or growth under stress conditions said method comprising
a) introducing into said plant and expressing a nucleic acid encoding an altered DELLA protein selected from GAI, RGL-1, 2 or 3 or their homologs or orthologues wherein the SUMOylation site is altered as described above or introducing into said plant and expressing a construct comprising a nucleic acid that encodes a GID1a receptor as defined in SEQ ID No. 10 but which comprises a substitution of one or more residue within the SIM site, for example of the conserved W or V residue or the K residue in the conserved SUMOylation site and
b) obtaining a progeny plant derived from the plant or plant cell of step a).

19. A method for producing a plant with altered root architecture or a method for increasing plant tolerance to nutrient deficient conditions, comprising preventing, decreasing or increasing SUMOylation of a AtARF19 or AtARF7 polypeptide as defined in SEQ ID No. 14 or 16 or a functional variant, homologue or orthologue thereof.

20. A method according to claim 19 comprising expressing a nucleic acid construct comprising a nucleic acid that encodes for a AtARF19 or AtARF7 polypeptide as defined in SEQ ID No. 14 or 16 or a functional variant, homologue or orthologue thereof but which comprises a substitution of one or more residue, for example K, in the conserved SUMOylation site in a plant.
